(19) United States
(12) Patent Application Publication
(10) Pub. No.: US 2022/0144809 A1
(43) Pub. Date: May 12, 2022

(54) COMPOUNDS AND METHODS FOR THE TARGETED DEGRADATION OF ANDROGEN RECEPTOR AND ASSOCIATED METHODS OF USE

(71) Applicant: Arvinas Operations, Inc., New Haven, CT (US)

(72) Inventors: Hanqing Dong, Madison, CT (US); Lawrence B. Snyder, Killingworth, CT (US); Jing Wang, Milford, CT (US)

(21) Appl. No.: 17/512,219

(22) Filed: Oct. 27, 2021

Related U.S. Application Data
(60) Provisional application No. 63/110,725, filed on Nov. 6, 2020.

(51) Int. Cl. C07D 401/14; A61K 45/06

(52) U.S. Cl. CPC C07D 401/14; A61K 45/06

(57) ABSTRACT

Bifunctional compounds, which find utility as modulators of androgen receptor (AR), are described herein. In particular, the bifunctional compounds of the present disclosure contain on one end a moiety that binds to the cereblon E3 ubiquitin ligase and on the other end a moiety which binds AR, such that the target protein is placed in proximity to the ubiquitin ligase to effect degradation (and inhibition) of target protein. The bifunctional compounds of the present disclosure exhibit a broad range of pharmacological activities associated with degradation/inhibition of target protein. Diseases or disorders that result from aberrant regulation of the target protein are treated or prevented with compounds and compositions of the present disclosure.

### CROSS-REFERENCE TO RELATED APPLICATIONS

The present application claims priority to and the benefit of U.S. Provisional Application No. 63/110,726, filed 6 Nov. 2020, titled COMPOUNDS AND METHODS FOR THE TARGETED DEGRADATION OF ANDROGEN RECEPTOR AND ASSOCIATED METHODS OF USE, which is incorporated by reference herein in its entirety for all purposes.

### INCORPORATION BY REFERENCE

All cited references are hereby incorporated herein by reference in their entirety, including U.S. patent application Ser. No. 14/686,640, filed on 14 Apr. 2015, published as U.S. Patent Application Publication No. 2015/0291562; and U.S. patent application Ser. No. 14/792,414, filed on 6 Jul. 2015, published as U.S. Patent Application Publication No. 2016/0058872; and U.S. patent application Ser. No. 15/953,108, filed on 13 Apr. 2018, published as U.S. Patent Application Publication No. 2018/0228907; and U.S. patent application Ser. No. 15/730,728, filed 11 Oct. 2017, issued as U.S. Pat. No. 10,584,101 on 10 Mar. 2020.

### FIELD OF THE INVENTION

The description provides hetero-bifunctional compounds comprising a target protein binding moiety and a E3 ubiquitin ligase binding moiety, and associated methods of use. The bifunctional compounds are useful as modulators of targeted ubiquitination of androgen receptor (AR), which is then degraded and/or inhibited.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part because they must disrupt protein-protein interactions. However, recent developments have provided specific ligands which bind to these ligases. For example, since the discovery of nutlins, the first small molecule E3 ligase inhibitors, additional compounds have been reported that target E3 ligases.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubiquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Bifunctional compounds such as those described in U.S. Patent Application Publications 2015/0291562 and 2014/0356322 (incorporated herein by reference), function to recruit endogenous proteins to an E3 ubiquitin ligase for ubiquitination and subsequent degradation in the proteasome degradation pathway. In particular, the publications cited above describe bifunctional or proteolysis-targeting chimeric (PROTAC®) protein degrader compounds, which find utility as modulators of targeted ubiquitination of a variety of polypeptides and other proteins, which are then degraded and/or inhibited by the bifunctional compounds.

Androgen Receptor (AR) belongs to a nuclear hormone receptor family that is activated by androgens, such as testosterone and dihydrotestosterone (Pharmacol. Rev. 2006, 58(4), 782-97; Vitam. Horm. 1999, 55:309-52). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1):1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers, including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12,823-824). Despite the development of effective targeted therapies, most patients develop resistance and the disease progresses. An alternative approach for the treatment of prostate cancer involves eliminating the AR protein. Because AR is a critical driver of tumorigenesis in many forms of prostate cancers, its elimination should lead to therapeutically beneficial response.

There exists an ongoing need in the art for effective treatments for diseases, especially cancer, prostate cancer, and Kennedy's Disease. However, non-specific effects, and the inability to target and modulate certain classes of proteins altogether, such as transcription factors, remain as obstacles to the development of effective anti-cancer agents. As such, an ongoing need exists in the art for effective treatments for AR related disease and discorders, e.g., cancer, prostate cancer, and Kennedy's Disease.

### SUMMARY

The present disclosure describes hetero-bifunctional compounds that function to recruit androgen receptor (AR) to an E3 ubiquitin ligase for targeted ubiquitination and subsequent proteasomal degradation, and methods of making and using the same. In particular, compounds as described herein preferentially bind to AR proteins. In addition, the description provides methods of using an effective amount of a compound of the invention, as described herein, for the treatment or amelioration of a disease condition or one or more symptoms thereof, such as Kennedy's Disease or cancer, e.g. prostate cancer.

As such, in one aspect the disclosure provides hetero-bifunctional compounds that comprise an E3 ubiquitin ligase binding moiety (i.e., a ligand for an E3 ubiquitin ligase (a “ULM” group)), and a protein targeting moiety that preferentially binds to AR, such that the AR protein is thereby preferentially placed in proximity to the ubiquitin ligase to effect ubiquitination and subsequent preferential degradation (and/or inhibition) of the AR protein. In a preferred embodiment, the ULM (ubiquitin ligase binding moiety) is a cereblon E3 ubiquitin ligase binding moiety (CLM). For example, the structure of the bifunctional compound can be depicted as:

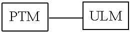

The respective positions of the PTM and ULM moieties (e.g., CLM), as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

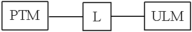

where PTM is a moiety that selectively or preferentially binds to an AR protein, L is a linker, e.g., a bond or a chemical linking group coupling PTM to ULM, and ULM is a cereblon E3 ubiquitin ligase binding moiety (CLM).

For example, the structure of the bifunctional compound can be depicted as:

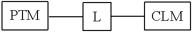

wherein: PTM is a moiety that selectively or preferentially binds to an AR protein; “L” is a linker (e.g. a bond or a chemical linking group) coupling the PTM and CLM; and CLM is cereblon E3 ubiquitin ligase binding moiety that binds to cereblon.

In certain embodiments, the compounds as described herein comprise multiple independently selected ULMs, multiple PTMs, multiple chemical linkers or a combination thereof.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is selected from thalidomide, lenalidomide, pomalidomide, analogs thereof, isosteres thereof, and derivatives thereof. Other contemplated CLMs are described in U.S. Patent Application Publication No. 2015/0291562, which is incorporated herein by reference in its entirety.

In certain embodiments, “L” is a bond. In additional embodiments, the linker “L” is a connector with a linear non-hydrogen atom number in the range of 1 to 20. The connector “L” can contain, but is not limited to one or more functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic or tricyclic moieties. Substitution with halogen, such as Cl, F, Br and I can be included in the linker. In the case of fluorine substitution, single or multiple fluorines can be included.

In certain embodiments, CLM is a derivative of piperidine-2,6-dione, where piperidine-2,6-dione can be substituted at the 3-position, and the 3-substitution can be bicyclic hetero-aromatics with the linkage as C—N bond or C—C bond. Examples of CLM can be, but are not limited to, pomalidomide, lenalidomide and thalidomide and their analogs.

In an additional aspect, the description provides therapeutic compositions comprising an effective amount of a compound as described herein, or a salt form thereof, and a pharmaceutically acceptable carrier. The therapeutic compositions can be used to trigger targeted degradation and/or inhibition of an AR protein in a patient or subject in need thereof, for example, an animal such as a human, and can be used for treating or ameliorating one or more disease states, conditions, or symptoms causally related to the AR protein, which treatment is accomplished through the degradation of the AR protein to control, stabilize or lower levels of protein of the AR protein in a patient or subject. In certain embodiments, the therapeutic compositions as described herein may be used to effectuate the degradation of AR for the treatment or amelioration of a disease, disorder or symptom, such as, e.g., an infection, an inflammatory or immunological disorder, or cancer.

In yet another aspect, the present disclosure provides a method of ubiquitinating an AR in a cell. In certain embodiments, the method comprises administering a hetero-bifunctional compound as described herein comprising a PTM that binds to an AR, and a CLM, preferably linked together through a chemical linker moiety, as described herein, to effectuate degradation of the AR protein. Though not wanting to be limited by theory, the inventors believe that, pursuant to the invention, poly-ubiquitination of the AR protein will occur when it is placed in proximity to the E3 ubiquitin ligase via use of the hetero-bifunctional compound, thereby triggering subsequent degradation of the ar protein via the proteasomal pathway, thereby controlling or reducing ar protein levels in cells of the subject. The control or reduction in AR protein levels afforded by the present disclosure provides treatment of a disease state, condition or at least one causally related symptom, as modulated through a lowering or stabilization of the amount of AR protein in cells of the subject.

In still another aspect, the description provides methods for treating or ameliorating a disease, condition, or symptom thereof in a subject or a patient, e.g., an animal such as a human, comprising administering to a subject in need thereof a composition comprising an effective amount, e.g., a therapeutically effective amount, of a hetero-bifunctional compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of an AR protein according to the disclosure in a biological system using compounds according to the present disclosure.

In another aspect, the description provides processes and intermediates for making a hetero-bifunctional compound of the invention capable of targeted ubiquitination and degradation of an AR protein according to the disclosure in a cell.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features and advantages of the disclosure will become apparent from the following detailed description taken in conjunction with the accompanying FIGURES showing illustrative embodiments of the disclosure.

FIGS. 1A and 1B. Illustration of general principle for the functioning of hetero-bifunctional protein degrading compounds as described herein. FIG. 1A. Exemplary hetero-bifunctional protein degrading compounds comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. FIG. 1B Illustrates the functional use of the hetero-bifunctional protein degrading compounds (commercially known as PROTAC® brand compounds) as described herein. Briefly, the ULM (triangle) recognizes and binds to a specific E3 ubiquitin ligase, and the PTM (large rectangle) binds and recruits a target protein bringing it into close proximity to the E3 ubiquitin ligase. Typically, the E3 ubiquitin ligase is complexed with an E2 ubiquitin-conjugating protein (E2), and either alone or via the E2 protein catalyzes attachment of multiple ubiquitin molecules (black circles) to a lysine on the target protein via an isopeptide bond. The poly-ubiquitinated protein (far right) has thereby been targeted for degradation by the proteosomal machinery of the cell.

### DETAILED DESCRIPTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, and other references mentioned herein are expressly incorporated by reference in their entirety.

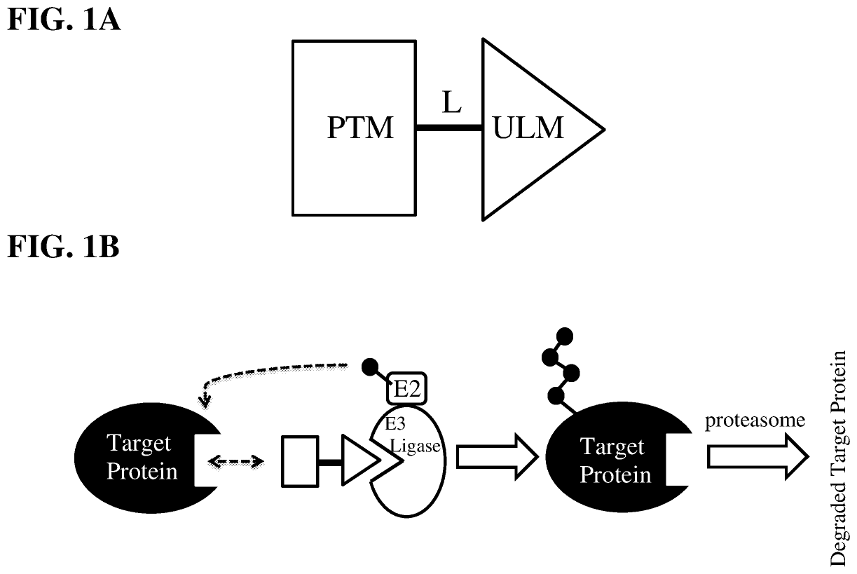

Presently described are compounds, compositions and methods that relate to the surprising and unexpected discovery that an E3 ubiquitin ligase (e.g., a cereblon E3 ubiquitin ligase) ubiquitinates the androgen receptor (AR) protein once the E3 ubiquitin ligase and the AR protein are placed in proximity via a bifunctional compound that binds both the E3 ubiquitin ligase and the AR protein. Accordingly the present disclosure provides compounds and compositions comprising an E3 ubiquitin ligase binding moiety (“ULM”) coupled by a bond or chemical linking group (L) to a protein targeting moiety (“PTM”) that targets the AR protein, which results in the ubiquitination of the AR protein, and which leads to degradation of the AR protein by the proteasome (see FIG. 1).

In one aspect, the description provides compounds in which the PTM preferably binds the AR protein. The present disclosure also provides a library of compositions and the use thereof to produce targeted degradation of the AR protein in a cell.

In certain aspects, the present disclosure provides hetero-bifunctional compounds which comprise a ligand, e.g., a small molecule ligand (i.e., having a molecular weight of below 2,000, 1,000, 500, or 200 Daltons), which is capable of binding to an E3 ubiquitin ligase, such as cereblon. The compounds also comprise a small molecule moiety that is capable of binding to AR in such a way that the AR protein is placed in proximity to the ubiquitin ligase to effect ubiquitination and degradation (and/or inhibition) of the AR protein. “Small molecule” means, in addition to the above, that the molecule is non-peptidyl, that is, it is not considered a peptide, e.g., comprises fewer than 4, 3, or 2 amino acids. In accordance with the present description, each of the PTM, ULM and hetero-bifunctional molecule is a small molecule.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value in the range, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods or processes described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time-varied administration (administration of one or more therapeutic agents at a time different from that of the administration of an additional therapeutic agent or agents), as long as the two or more therapeutic agents are present in the patient to some extent, preferably at effective amounts, at the same time. In certain preferred aspects, one or more of the hetero-bifunctional compounds described herein are coadministered with at least one additional bioactive agent, e.g., an anticancer agent. In particularly preferred aspects, the co-administration of such compounds results in synergistic activity and/or therapy such as, e.g., anticancer activity.

The term “compound”, as used herein, unless otherwise indicated, refers to any specific hetero-bifunctional compound disclosed herein, pharmaceutically acceptable salts and solvates thereof, and deuterated forms of any of the aforementioned molecules, where applicable. Deuterated compounds contemplated are those in which one or more of the hydrogen atoms contained in the drug molecule have been replaced by deuterium. Such deuterated compounds preferably have one or more improved pharmacokinetic or pharmacodynamic properties (e.g., longer half-life) compared to the equivalent “undeuterated” compound.

The term “ubiquitin ligase” refers to a family of proteins that facilitate the transfer of one or more ubiquitins to a specific substrate protein. Addition of a chain of several ubiquitins (poly-ubiquitination) targets the substrate protein for degradation. For example, cereblon is an E3 ubiquitin ligase that alone, or in combination with an E2 ubiquitin-conjugating enzyme, can ultimately cause the attachment of a chain of four ubiquitins to a lysine residue on the target protein, thereby targeting the protein for degradation by the proteasome. The ubiquitin ligase is involved in poly-ubiquitination such that a first ubiquitin is attached to a lysine on the target protein; a second ubiquitin is attached to the first; a third is attached to the second, and a fourth is attached to the third. Such poly-ubiquitination marks proteins for degradation by the proteasome.

The term “patient” or “subject” is used throughout the specification to describe an animal, preferably a human or a domesticated animal, to whom treatment, including prophylactic treatment, with the compositions according to the present disclosure is provided. For treatment of those diseases, conditions or symptoms that are specific for a specific animal, such as a human patient, the term “patient” refers to that specific animal, including a domesticated animal such as a dog or cat, or a farm animal such as a horse, cow, sheep, etc. In general, in the present disclosure, the terms “patient” and “subject” refer to a human patient unless otherwise stated or implied from the context of the use of the term.

The terms “effective” and “therapeutically effective” are used to describe an amount of a compound or composition which, when used within the context of its intended use, and either in a single dose or, more preferably after multiple doses within the context of a treatment regimen, effects an intended result such as an improvement in a disease or condition, or amelioration or reduction in one or more symptoms associated with a disease or condition. The terms “effective” and “therapeutically effective” subsume all other “effective amount” or “effective concentration” terms, which are otherwise described or used in the present application.

Compounds and Compositions

In one aspect, the description provides hetero-bifunctional compounds comprising an E3 ubiquitin ligase binding moiety (“ULM”) that is a cereblon E3 ubiquitin ligase binding moiety (a “CLM”), The CLM is covalently coupled to a protein targeting moiety (PTM) that binds to the protein, which coupling is either directly by a bond or via a chemical linking group (L) according to the structure:

PTM-L-CLM (A)

wherein L is the bond or chemical linking group, and PTM is a protein targeting moiety that binds to the protein AR, where the PTM is a small molecule AR targeting moiety. The term CLM is inclusive of all cereblon binding moieties.

In any of the aspects or embodiments, the CLM demonstrates a half maximal inhibitory concentration (IC₅₀) for the E3 ubiquitin ligase (e.g., cereblon E3 ubiquitin ligase) of less than about 200 μM. The IC₅₀ can be determined according to any suitable method known in the art, e.g., a fluorescent polarization assay.

In certain embodiments, the hetero-bifunctional compounds described herein demonstrate an IC₅₀ or a half maximal degradation concentration (DC₅₀) of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 μM.

The term “alkyl” shall mean within its context a linear, branch-chained or cyclic fully saturated hydrocarbon radical, preferably a C₁-C₁₀, preferably a C₁-C₆, or more preferably a C₁-C₃alkyl group, which may be optionally substituted with any suitable functional group or groups. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I).

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one C—C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other suitable functional group) which may be further substituted with a polyethylene glycol chain (of from 1 to 10, preferably 1 to 6, or more preferably 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (e.g., methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than one substituent occurs, each substituent is selected independent of another substituent) one or more substituents (independently up to five substituents, preferably up to three substituents, more preferably 1 or 2 substituents on a moiety in a compound according to the present disclosure and may include substituents which themselves may be further substituted) at a carbon (or nitrogen) position anywhere on a molecule within context, and includes as possible substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl, for example benzyl or benzoyl), alkoxy group (preferably, C₁-C₆ alkyl or aryl, including phenyl and substituted phenyl), thioether (preferably, C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), halogen (preferably, F or C₁), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which are preferably independently substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ together is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)ₘ— or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a side chain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂), COOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical (e.g., a 5-16 membered ring) having a single ring (e.g., benzene, phenyl, benzyl, or 5, 6, 7 or 8 membered ring) or condensed rings (e.g., naphthyl, anthracenyl, phenanthrenyl, 10-16 membered ring, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, C₁) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group, via a linker group), and/or at least one of F, C₁, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methyl substituted isoxazole, an optionally substituted oxazole including a methyl substituted oxazole, an optionally substituted thiazole including a methyl substituted thiazole, an optionally substituted isothiazole including a methyl substituted isothiazole, an optionally substituted pyrrole including a methyl substituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methyl substituted triazole group, an optionally substituted pyridine group, including a halo-(preferably, F) or methyl substituted pyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to a 5-16 membered heteroaryl (e.g., 5, 6, 7 or 8 membered monocylic ring or a 10-16 membered heteroaryl having multiple condensed rings), an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

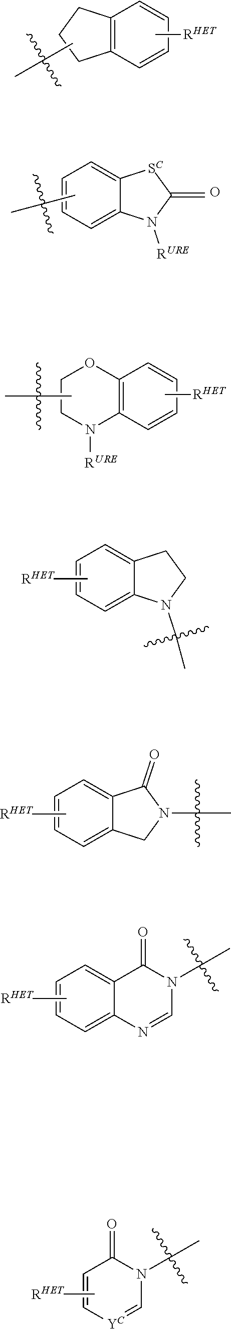

wherein:
S^{c} is CHR^{SS}, NR^{URE}, or O;
R^{HET} is H, CN, NO₂, halo (preferably C₁ or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidiiyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, SO-heteroaryl, —SO2-alkyl, —SO2-substituted alkyl, —SO2-aryl, oxo (═O), and —SO2-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

Exemplary CLMs

In any aspect or embodiment described herein, the description provides CLMs or the ULMs useful for binding and recruiting cereblon. In any aspect or embodiment described herein, the CLM or the ULM is represented by the chemical structure:

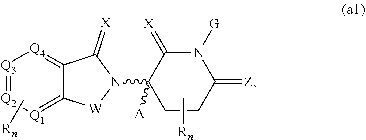

wherein:
W is independently selected from the group CH₂, O, CHR, C═O, SO₂, NH, N, optionally substituted cyclopropyl group, optionally substituted cyclobutyl group, and N-alkyl;
each X is independently selected from the group absent, O, S and CH₂;
Z is independently selected from the group absent, O, S, and CH₂ except that both X and Z cannot be CH₂ or absent;
G is selected from the group H, optionally substituted linear or branched alkyl, OH, R′OCOOR, R′OCONRR″, CH₂-heterocyclyl optionally substituted with R′, and benzyl optionally substituted with R′;
Q1-Q4 each independently represent a N or a C substituted with a group independently selected from H or R;
A is independently selected from the group H, optionally substituted linear or branched alkyl, cycloalkyl, Cl and F;
n is an integer from 1 to 10 (e.g., 1-4, 1, 2, 3, 4, 5, 6, 7, 8, 9, or 10);
R comprises, but is not limited to: H, —C(═O)R′ (e.g., a carboxy group), —CONR′R″ (e.g., an amide group), —OR′ (e.g., OH), —NR′R″ (e.g., an amine group), —SR′, —SO₂R′, —SO₂NR′R″, —CR′R″—, —CR′NR′R″—, (—CR′O)_{n′}R″, optionally substituted heterocyclyl, optionally substituted aryl, (e.g., an optionally substituted C5-C7 aryl), optionally substituted alkyl-aryl (e.g., an alkyl-aryl comprising at least one of an optionally substituted C1-C6 alkyl, an optionally substituted C5-C7 aryl, or combinations thereof), optionally substituted heteroaryl, optionally substituted alkyl (e.g., a C1-C6 linear or branched alkyl optionally substituted with one or more halogen, cycloalkyl (e.g., a C3-C6 cycloalkyl), or aryl (e.g., C5-C7 aryl)), optionally substituted alkoxyl group (e.g., a methoxy, ethoxy, butoxy, propoxy, pentoxy, or hexoxy; wherein the alkoxyl may be substituted with one or more halogen, alkyl, haloalky, fluoroalkyl, cycloalkyl (e.g., a C₃-C₆ cycloalkyl), or aryl (e.g., C₅-C₇ aryl)), optionally substituted cycloalkyl, optionally substituted heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃, wherein at least one W, X, Y, Z, G, G′, R, R′, R″, Q1-Q4, or A is the point of attachment or is modified to be covalently joined to a PTM, a chemical linking group (L), a ULM, CLM, or combination thereof;
R′ and R″ are independently selected from H, optionally substituted linear or branched alkyl (e.g., optionally substituted linear or branched C1-6 alkyl), optionally substituted cycloalkyl (e.g., optionally substituted 3-7 membered cycloalkyl), optionally substituted aryl (e.g., optionally substituted 5-7 membered aryl), optionally substituted heteroaryl (e.g., optionally substituted 5-7 membered heteraryl), optionally substituted heterocyclic (e.g., optionally substituted 3-7 membered heterocyclic), —C(═O)R, optionally substituted heterocyclyl (e.g., optionally substituted 3-7 membered heterocyclyl);
n′ is an integer from 1-10 (e.g. 1-4, 1, 2, 3, 4, 5, 6, 7, 8, 9, or 10); and
[symbol] represents a bond that may be stereospecific ((R) or (S)) or non-stereospecific.

In any aspect or embodiment described herein, the CLM or the ULM has a chemical structure represented by Formula (a1):

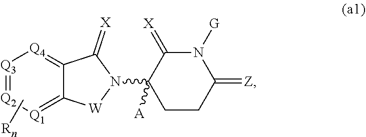

wherein:
W is selected from the group consisting of CH₂, O, C═O, NH, and N-alkyl;
each X is O or S;
Z is O or S;
G is H or an unsubstituted linear or branched C₁₋₃ alkyl;
Q₁, Q₂, Q₃, and Q₄ represent a N or a C substituted with a group selected from H and R; A is independently selected from the group H, unsubstituted linear or branched C₁₋₃ alkyl (e.g., optionally substituted methyl or ethyl), cycloalkyl (e.g., a C3-4 cycloalkyl), Cl and F;
n is an integer from 1 to 4 (e.g., 1, 2, 3, or 4);
R is selected from the group consisting of H, NH₂, an unsubstituted or substituted linear or branched C₁₋₄ alkyl (e.g., optionally substituted methyl or ethyl), —OR′, —Cl, —F, —Br, —I, —CF₃, —CN, and —NO₂, wherein an R is the point of attachment or an R is modified to be covalently joined to the chemical linking group (L);
R′ is independently selected from the group consisting of H and an unsubstituted or substituted C₁₋₃ alkyl (e.g., optionally substituted methyl or ethyl); and
[symbol] represents a bond that may be stereospecific ((R) or (S)) or non-stereospecific.

In any aspect or embodiment described herein, the CLM or the ULM has a chemical structure represented by:

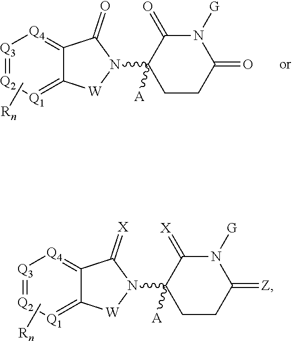

wherein:
W is selected from the group consisting of CH₂, C═O, NH, and N-alkyl;
each X is O or S;
Z is O or S;
G is H or an unsubstituted linear or branched C₁₋₃ alkyl;
Q₁, Q₂, Q₃, and Q₄ represent a N or a C substituted with a group selected from H and R; A is independently selected from the group H, unsubstituted linear or branched C₁₋₃ alkyl (e.g., optionally substituted methyl or ethyl), Cl and F;
n is an integer from 1 to 4 (e.g., 1, 2, 3, or 4);
R is selected from the group consisting of H, NH₂, an unsubstituted or substituted linear or branched C₁₋₄ alkyl (e.g., optionally substituted methyl or ethyl), —OR′, —Cl, —F, —Br, —CF₃, and —CN, wherein an R is the point of attachment or an R is modified to be covalently joined to the chemical linking group (L);
R′ is independently selected from the group consisting of H and an unsubstituted or substituted C₁₋₄ alkyl (e.g., optionally substituted methyl or ethyl); and
[symbol] represents a bond that may be stereospecific ((R) or (S)) or non-stereospecific.

In any aspect or embodiment described herein, the CLM or the ULM has the chemical structure of Formula (a1′):

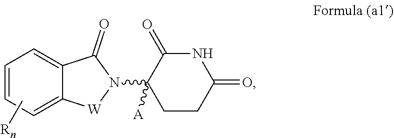

wherein:
W is independently selected from the group CH₂, C═O, NH, and N-alkyl;
A is selected from a H, or optionally substituted linear or branched C₁-C₆ alkyl (e.g., optionally substituted methyl or ethyl);
n is an integer from 1 to 4 (e.g., 1, 2, 3, or 4);
R is independently selected from a H, OH, NH₂, Cl, —F, —Br, optionally substituted linear or branched C₁-C₄ alkyl (e.g., optionally substituted methyl or ethyl), optionally substituted linear or branched C₁-C₄ alkoxy (e.g., optionally substituted methoxy or ethoxy), wherein an R is modified to be covalently joined to a chemical linking group (L); and
[symbol] represents a bond that may be stereospecific ((R) or (S)) or non-stereospecific.

In any aspect or embodiment described herein, the CLM or the ULM has the chemical structure of Formula (a1′):

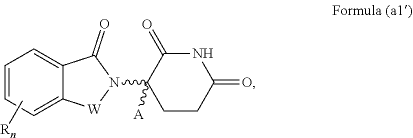

wherein:
W is independently selected from the group CH₂ and C═O;
A is selected from a H or methyl, preferably H;
n is 1 or 2;
each R is independently selected from a H, OH, NH₂, Cl, —F, —Br, optionally substituted linear or branched C₁₋₃ alkyl (e.g., an optionally substituted methyl or ethyl), optionally substituted linear or branched C₁₋₃ alkoxy (e.g., an optionally substituted methoxy or ethoxy), wherein an R is the point of attachment or an R is modified to be covalently joined to the chemical linking group (L); and
[symbol] represents a bond that may be stereospecific ((R) or (S)) or non-stereospecific.

In any aspect or embodiment described herein, the CLM or the ULM is selected from the group consisting of:

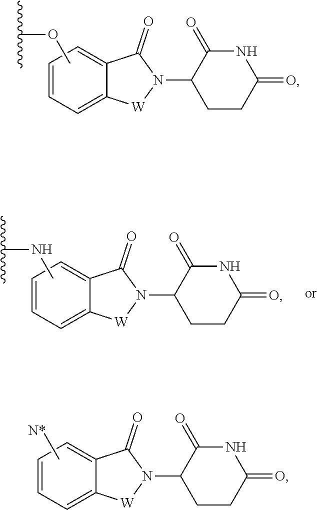

wherein:
W is C═O or CH₂;
N* is a nitrogen atom that is shared with the PTM or linker (L) (e.g., a heteroatom shared with an optionally substituted heterocylyl of the linker (L) or PTM); and

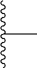

indicates the point of attachment of the CLM or the ULM to the linker (L) or PTM.

In any aspect or embodiment described herein, the CLM or the ULM is selected from the group consisting of:

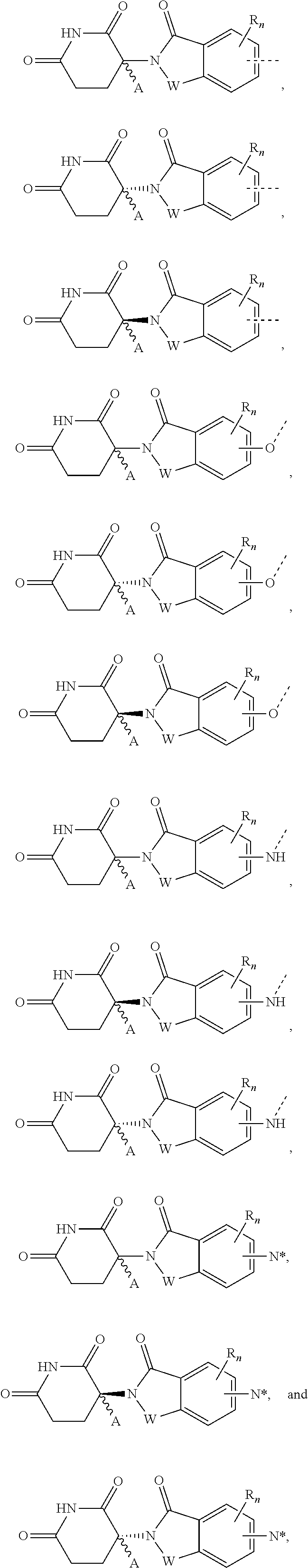

wherein:
[symbol] of the CLM indicates the point of attachment with the chemical linking group;
N* is a nitrogen atom that is shared with the chemical linking group;
n is 1 or 2; and
the other variables are as defined in any aspect or embodiment described herein,
wherein an R can be the point of attachment or an R can be modified to be covalently joined to the chemical linking group (L).

In any aspect or embodiment described herein, the CLM or the ULM is selected from the group consisting of:

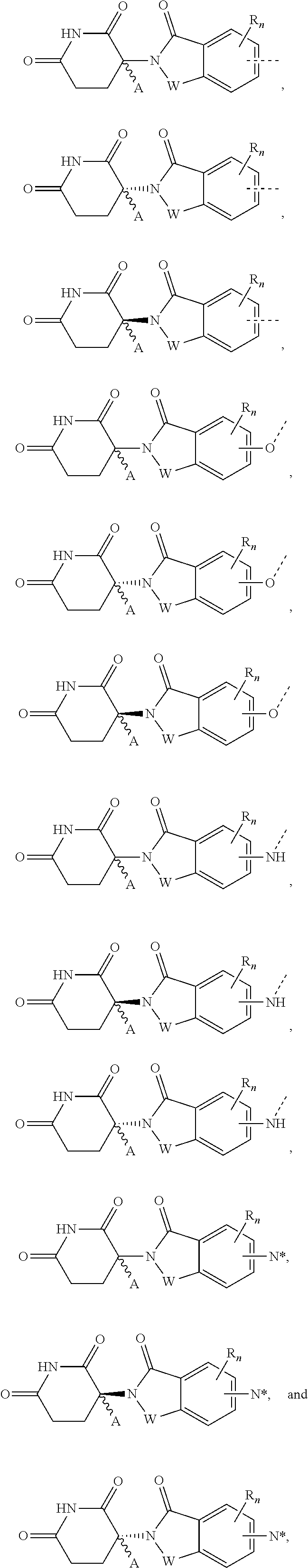

wherein:
[symbol] of the CLM indicates the point of attachment with the chemical linking group;
N* is a nitrogen atom that is shared with the chemical linking group; and
the other variables are as defined in any aspect or embodiment described herein,
wherein an R can be the point of attachment or an R can be modified to be covalently joined to the chemical linking group (L).

In any aspect or embodiment described herein, the CLM or the ULM is selected from the group consisting of:

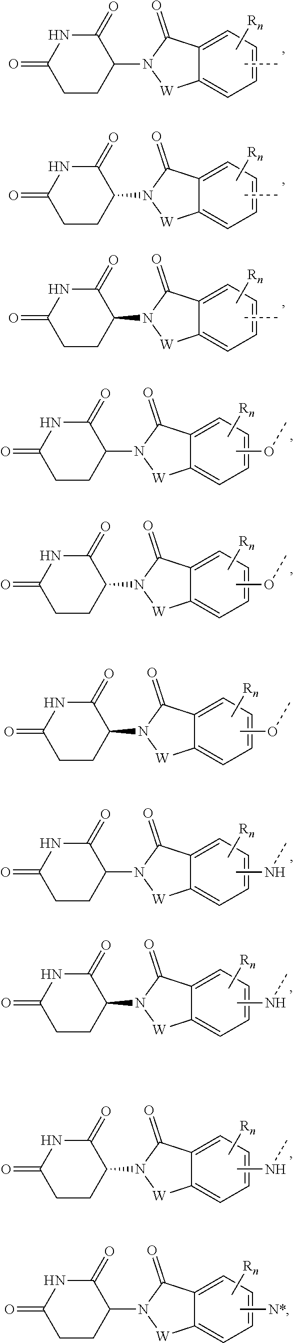

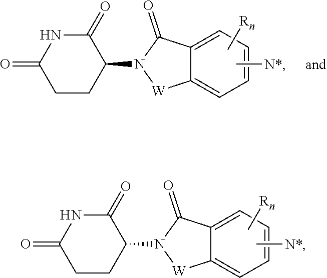

wherein:
[symbol] of the CLM indicates the point of attachment with the chemical linking group;
N* is a nitrogen atom that is shared with the chemical linking group;
n is 1 or 2; and
the other variables are as defined in any aspect or embodiment described herein,
wherein an R can be the point of attachment or an R can be modified to be covalently joined to the chemical linking group (L).

In any aspect or embodiment described herein, the CLM or the ULM is selected from the group consisting of:

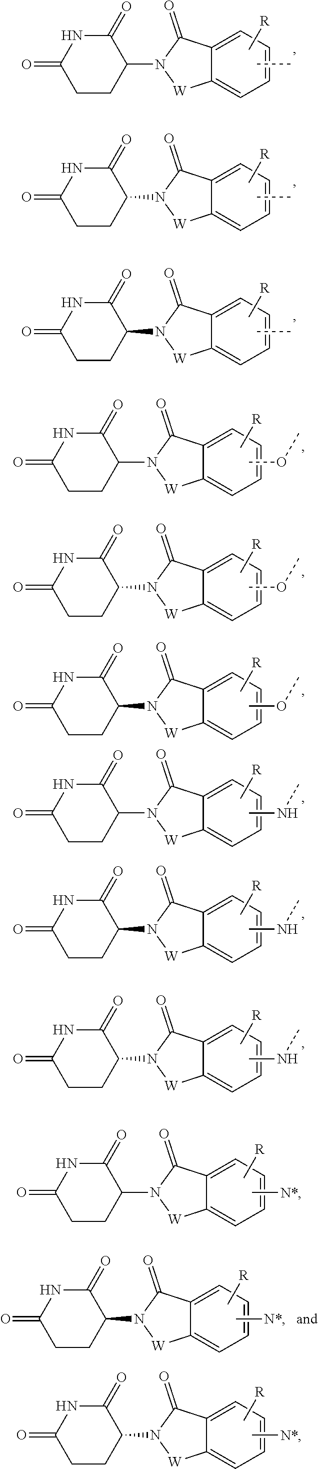

wherein:
[symbol] of the CLM indicates the point of attachment with the chemical linking group;
N* is a nitrogen atom that is shared with the chemical linking group; and
the other variable are as defined in any aspect or embodiment described herein,
wherein an R can be the point of attachment or an R can be modified to be covalently joined to the chemical linking group (L)

In any aspect or embodiment described herein, R is selected from: H, O, OH, NH₂, C1-C6 alkyl, C1-C6 alkoxy, -alkyl-aryl (e.g., an -alkyl-aryl comprising at least one of C1-C6 alkyl, C5-C7 aryl, or a combination thereof), aryl (e.g., C5-C7 aryl), amine, amide, or carboxy).

In any aspect or embodiment described herein, at least one R (e.g. an R group selected from the following H, O, OH, NH₂, C1-C6 alkyl, C1-C6 alkoxy, -alkyl-aryl (e.g., an -alkyl-aryl comprising at least one of C1-C6 alkyl, C5-C7 aryl, or a combination thereof), aryl (e.g., C5-C7 aryl), amine, amide, or carboxy) or W is the point of attachment or is modified to be covalently joined to a PTM, a chemical linker group (L), a ULM, a CLM, or a combination thereof

In any aspect or embodiment described herein, the W, X, Z, G, R, R′, R″, Q1-Q4, and A can independently be covalently coupled to a linker and/or a linker to which is attached one or more PTM, ULM, or CLM groups.

In any of the aspects or embodiments described herein, n is an integer from 1 to 4, and each R is independently selected functional groups or atoms, for example, O, OH, —Cl, —F, C1-C6 alkyl, C1-C6 alkoxy, -alkyl-aryl (e.g., an -alkyl-aryl comprising at least one of C1-C6 alkyl, C5-C7 aryl, or a combination thereof), aryl (e.g., C5-C7 aryl), amine, amide, or carboxy, on the aryl or heteroaryl of the CLM, and optionally, one of which is covalently joined to, or modified to be covalently joined to, a PTM, a chemical linker group (L), a ULM, CLM or combination thereof.

More specifically, non-limiting examples of CLMs include those shown below as well as those “hybrid” molecules that arise from the combination of one or more of the different features shown in the molecules below wherein at least one R is modified to be or the point that is covalently joined to a PTM, a chemical linking group (L), a ULM, CLM, or combination thereof.

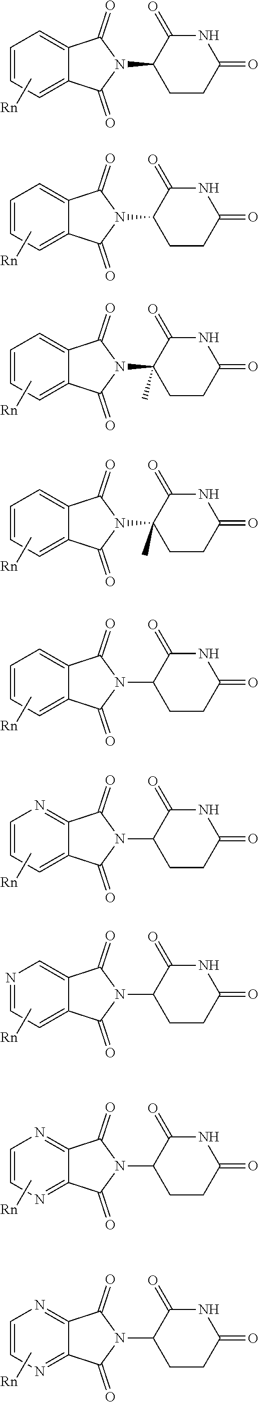

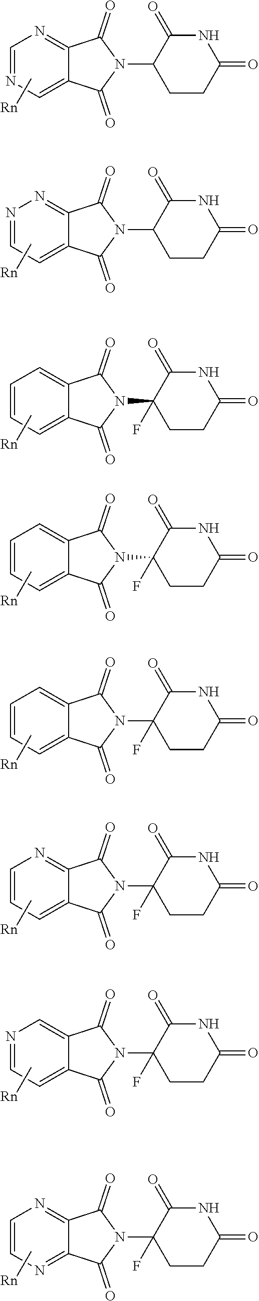

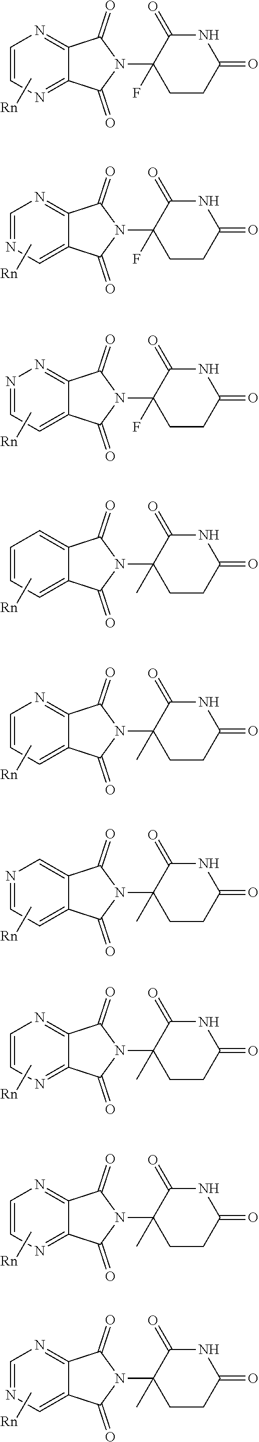

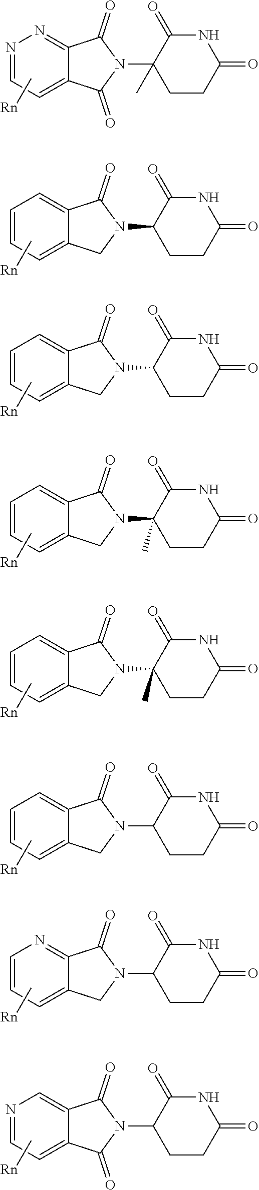

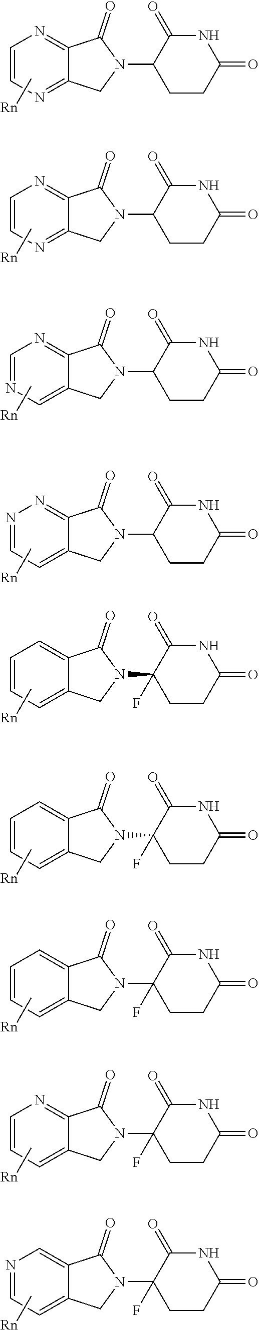

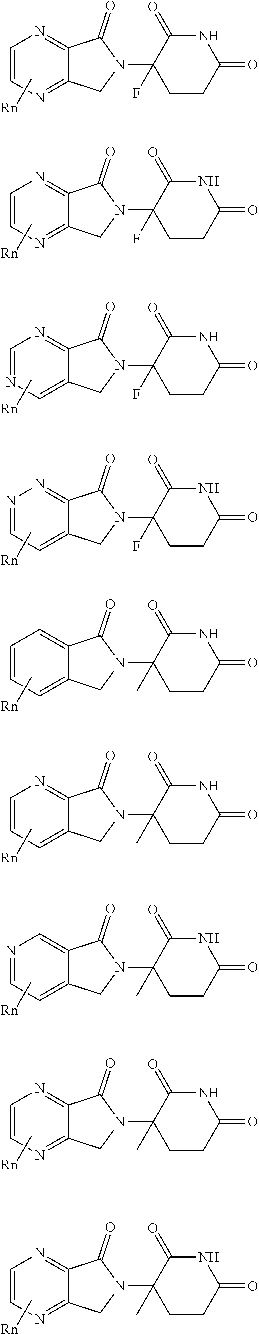

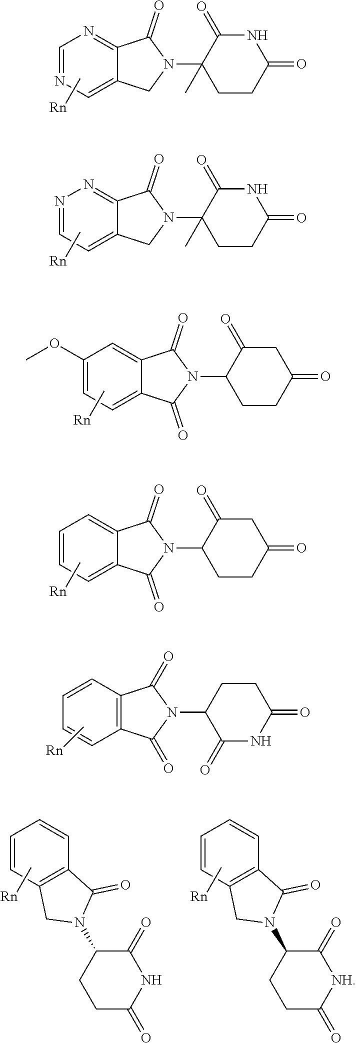

In any aspect or embodiment described herein, the CLM is covalently joined to a chemical linker group (L) via an R group or a Q group (such as, Q₁, Q₂, Q₃, or Q₄).

In any aspect or embodiment described herein, the CLM is covalently joined to a chemical linker group (L) via an R group.

In any aspect or embodiment described herein, the R can be covalently coupled to a linker and/or a linker to which is attached a PTM group.

In any aspect or embodiment described herein, the Q₁, Q₂, Q₃, or Q₄ can be covalently coupled to a linker and/or a linker to which is attached a PTM group.

In any aspect or embodiment described herein, R is modified to be covalently joined to the linker group (L).

In any aspect or embodiment described herein, R represents the point of the CLM that is covalently joined to the linker group (L).

In any aspect or embodiment described herein, “CLM” can be an imide that binds to cereblon E3 ligase. In any aspect or embodiment described herein, the imides and linker attachment point can be, but not be limited to, one of the following structures (e.g., any of the following attachment points can be utilized for any CLM chemical structure described herein):

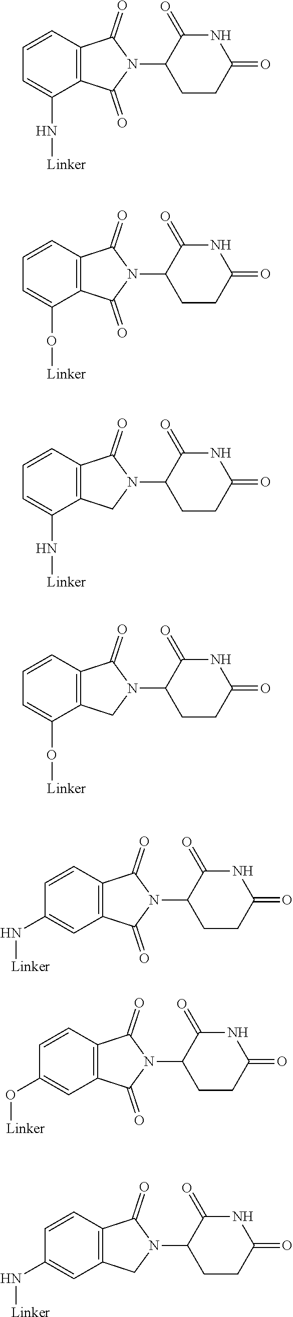

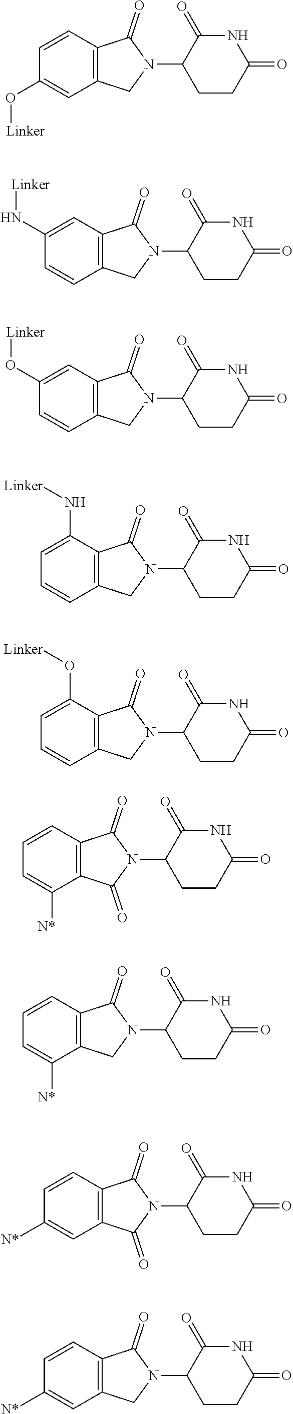

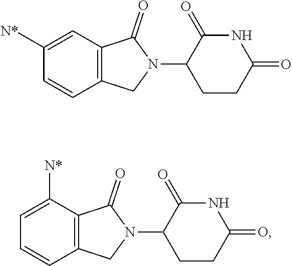

wherein:
N* is a nitrogen atom that is shared with the chemical linker group.

In any aspect or embodiment described herein, the ULM is selected from the group consisting of:

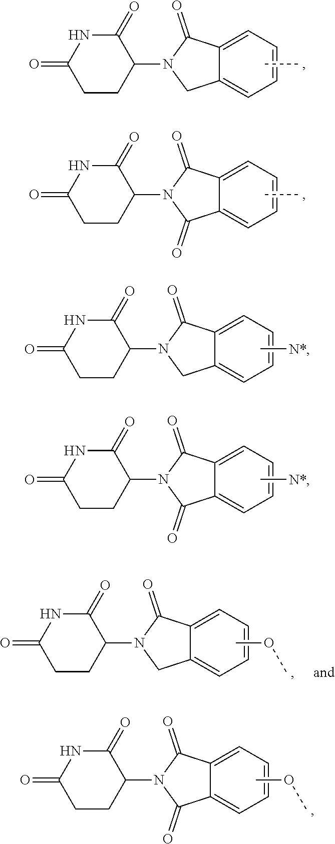

wherein: [symbol]
of the ULM indicates the point of attachment with a linker group or a PTM; and
N* is a nitrogen atom that is shared with the chemical linker group.

Exemplary Linkers

In any aspect or embodiment described herein, the compounds as described herein include a PTM chemically linked to a ULM (e.g., CLM) via a chemical linker (L). In certain embodiments, the linker group L comprises one or more covalently connected structural units (e.g., -A^{L}₁ . . . (A^{L})_{q}- or -(A^{L})_{q}-), wherein A^{L}₁ is a group coupled to PTM, and (A^{L})_{q} is a group coupled to ULM.

In any aspect or embodiment described herein, the linker (L) to a ULM (e.g., CLM) connection is a stable L-ULM connection. For example, in certain embodiments, when a linker (L) and a ULM are connected via a heteroatom (e.g., N, O, S), any additional heteroatom, if present, is separated by at least a carbon atom (e.g., —CH₂—), such as with an acetal or aminal group. By way of further example, in certain embodiments described herein, when a linker (L) and a ULM are connected via a heteroatom, the heteroatom is not part of an ester.

In any aspect or embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to both the PTM and the ULM, and provides for binding of the PTM to the protein target and the ULM to an E3 ubiquitin ligase to effectuate target protein ubiquitination.

In any aspect or embodiment described herein, the linker group L is a bond or a chemical linker group represented by the formula -(A^{L})_{q}-, wherein A is a chemical moiety and q is an integer from 1-30 (e.g., 1, 2, 3, 4, 5, 6, 7, 8, 9, 10, 11, 12, 13, 14, 15, 16, 17, 18, 19, 20, 21, 22, 23, 24, or 25), and wherein L is covalently bound to both the PTM and the ULM, and provides for binding of the PTM to the protein target and the ULM to an E3 ubiquitin ligase in sufficient proximity to result in target protein ubiquitination.

In any aspect or embodiment described herein, the linker group L is -(A^{L})_{q}-, wherein:
(A^{L})_{q} is a group which connects a ULM (e.g., CLM), to PTM;
q of the linker is an integer greater than or equal to 1;
each A^{L} is independently selected from the group consisting of, a bond, CR^{L1}R^{L2}, O, NR^{L3}, CO, CR^{L1}═CR^{L2}, C≡C, C₃₋₇cycloalkyl optionally substituted with 1-3 R^{L1} and/or R^{L2} groups, C₅-13 spirocycloalkyl optionally substituted with 1-5 R^{L1} and/or R^{L2} groups, C₃₋₇heterocyclyl optionally substituted with 1-3 R^{L1} and/or R^{L2} groups, C₅₋₁₃ spiroheterocyclyl optionally substituted with 1-5 R^{L1} and/or R^{L2} groups, 5-6 membered aryl optionally substituted with 1-3 R^{L1} and/or R^{L2} groups, and 5-6 membered heteroaryl optionally substituted with 1-3 R^{L1} and/or R^{L2} groups, where R^{L1} or R^{L2}, each independently are optionally linked to other groups to form cycloalkyl and/or heterocyclyl moiety, optionally substituted with 1-2 R^{L5} groups; and
R^{L1}, R^{L2}, R^{L3}, and R^{L5} are, each independently, H, halogen, C₁₋₈alkyl, OC₁₋₄alkyl, NHC₁₋₄alkyl, N(C₁₋₄alkyl)₂, OH, NH₂, CN, CF₃, CHF₂, CH₂F, or NO₂.

In certain embodiments, q is an integer greater than or equal to 1.

In certain embodiments, e.g., where q of the linker is greater than 2, (A^{L})_{q} is a group which is A^{L}₁ and (A^{L})_{q} wherein the linker couples a PTM to a ULM.

In certain embodiments, e.g., where q of the linker is 2, A^{L}₂ is a group which is connected to A^{L}₁ and to a ULM.

In certain embodiments, e.g., where q of the linker is 1, the structure of the linker group L is -A^{L}₁-, and A^{L}₁ is a group which connects a ULM moiety to a PTM moiety.

In any aspect or embodiment described herein, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(heterocycloalkyl)-, —NR(CH₂CH₂O)ₙ-(lower alkyl)-O—CH₂—, —NR(CH₂CH₂O)ₙ-(heterocycloalkyl)-O—CH₂—, —NR(CH₂CH₂O)ₙ-Aryl-O—CH₂—, —NR(CH₂CH₂O)ₙ-(heteroaryl)-O—CH₂—, —NR(CH₂CH₂O)ₙ-(cyclo alkyl)-O-(heteroaryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocyclyl)-CH₂, —NR(CH₂CH₂)ₙ-(heterocyclyl)-(heterocyclyl)-CH₂, and —N(R1R2)-(heterocyclyl)-CH₂,
wherein:
   n of the linker can be 0 to 10;
   R of the linker can be H, or lower alkyl; and
   R1 and R2 of the linker can form a ring with the connecting N.

In any aspect or embodiment described herein, the linker (L) includes an optionally substituted C₁-C₂₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, or C₂₀ alkyl, and including all implied subranges, e.g., C1-C10, C1-C15; C2-C10, C2-C15, etc.), wherein each carbon is optionally independently substituted or replaced with (1) a heteroatom selected from N, or O, atoms that has an appropriate number of hydrogens, substitutions, or both to complete valency, (2) an optionally substituted cycloalkyl (e.g., optionally substituted 3-7 membered cycloalkyl) or bicyclic cycloalkly (e.g., optionally substituted 5-10 membered bicyclic cycloalkyl), (3) an optionally substituted heterocyloalkyl (e.g., optionally substituted 3-7 membered heterocycloalkyl) or bicyclic heterocyloalkyl (e.g., optionally substituted 5-10 membered bicyclic heterocycloalkyl), (4) an optionally substituted aryl (e.g., optionally substitute 5-6 membered aryl) or bicyclic aryl (e.g., optionally substituted 9-12 membered bicyclic aryl), or (5) optionally substituted heteroaryl (e.g., optionally substitute 5-6 membered heteroaryl) or bicyclic heteroaryl (e.g., optionally substituted 9-12 membered bicyclic heteroaryl). In any aspect or embodiment described herein, the linker (L) does not have heteroatom-heteroatom bonding (e.g., no heteroatoms are covalently linked or adjacently located).

In any aspect or embodiment described herein, the linker (L) includes an optionally substituted C₁-C₂₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, or C₂₀ alkyl), wherein:
each carbon is optionally independently substituted or replaced with CR^{L1}R^{L2}, O, NR^{L3}, CO, CR^{L1}═CR^{L2}, C≡C, C₃₋₇cycloalkyl optionally substituted with 1-3 R^{L1} and/or R^{L2} groups, C₅₋₁₃ spirocycloalkyl optionally substituted with 1-5 R^{L1} and/or R^{L2} groups, C₃₋₇ heterocyclyl optionally substituted with 1-3 R^{L1} and/or R^{L2} groups, C₅₋₁₃ spiroheterocyclyl optionally substituted with 1-5 R^{L1} and/or R^{L2} groups, 5-6 membered aryl optionally substituted with 1-3 R^{L1} and/or R^{L2} groups, 5-6 membered heteroaryl optionally substituted with 1-3 R^{L1} and/or R^{L2} groups, where R^{L1} or R^{L2}, each independently are optionally linked to other groups to form a cycloalkyl and/or a heterocyclyl moiety, optionally substituted with 1-2 R^{L5} groups; and
R^{L1}, R^{L2}, R^{L3}, and R^{L5} are, each independently, H, halogen, C₁₋₄alkyl, OC₁₋₄alkyl, NHC₁₋₄alkyl, N(C₁₋₄alkyl)₂, OH, NH₂, CN, CF₃, CHF₂, CH₂F, or NO₂.

In any aspect or embodiment described herein, the linker group is optionally substituted an optionally substituted C₁-C₁₅ alkyl (e.g., C₁, C₂, C₃, C₄, C₅, C₆, C₇, C₈, C₉, C₁₀, C₁₁, C₁₂, C₁₃, C₁₄, C₁₆, C₁₇, Cis, C₁₉, or C₂₀ alkyl, and including all implied subranges, e.g., C1-C10, C1-C15, etc.), wherein each carbon atom optionally substituted or replaced with: a O, N, S, P or Si atom that has an appropriate number of hydrogens, substitutions (e.g., OH, halo, alkyl, methyl, ethyl, haloalkyl, hydroxyalkyl, alkoxy, methoxy, etc.), or both to complete valency; an optionally substituted aryl (e.g., an optionally substituted C5 or C6 aryl) or bicyclic aryl (e.g, an optionally substituted C5-C20 bicyclic heteroaryl); an optionally substituted heteroaryl (e.g., an optionally substituted C5 or C6 heteroaryl) or bicyclic heteroaryl (e.g., an optionally substituted heteroaryl or bicyclic heteroaryl having one or more heteroatoms selected from N, O, S, P, and Si that has an appropriate number of hydrogens, substitutions (e.g., OH, halo, alkyl, methyl, ethyl, haloalkyl, hydroxyalkyl, alkoxy, methoxy, etc.), or both to complete valency); an optionally substituted C1-C6 alkyl; an optionally substituted C1-C6 alkenyl; an optionally substituted C1-C6 alkynyl; an optionally substituted cycloalkyl (e.g., an optionally substituted C3-C7 cycloalkyl) or bicyclic cycloalkyl (e.g., an optionally substituted C5-C20 bicyclic cycloalkyl); or an optionally substituted heterocycloalkyl (e.g., an optionally substituted 3-, 4-, 5-, 6-, or 7-membered heterocyclic group) or bicyclicheteroalkyl (e.g., an optionally substituted heterocycloalkyl bicyclicheteroalkyl having one or more heteroatoms selected from N, O, S, P, or Si atoms that has an appropriate number of hydrogens, substitutions (e.g., OH, halo, alkyl, methyl, ethyl, haloalkyl, hydroxyalkyl, alkoxy, methoxy, etc.), or both to complete valency). In any aspect or embodiment described herein, the optionally substituted alkyl linker is optionally substituted with one or more OH, halo, linear or branched C1-C6 alkyl (such as methyl or ethyl), linear or branched C1-C6 haloalkyl, linear or branched C1-C6 hydroxyalkyl, or linear or branched C1-C6 alkoxy (e.g., methoxy).

In any aspect or embodiment described herein, the linker (L) does not have heteroatom-heteroatom bonding (e.g., no heteroatoms are covalently linked or adjacently located).

In any aspect or embodiment described herein, the linker (L) includes about 1 to about 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) alkylene glycol units that are optionally substituted, wherein carbon or oxygen may be substituted with a N atom with an appropriate number of hydrogens to complete valency.

In any aspect or embodiment described herein, the linker (L) comprises or is the chemical structure:

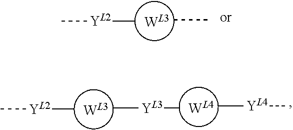

wherein:
Y^{L2} is a bond, O, or a unsubstituted or substituted linear or branched C1-C10 alkyl (C₁, C₂, C₃, C₄, C₅, C₆, C₇, C₈, C₉, or C₁₀ alkyl), wherein one or more C atoms are optionally replaced with O and each carbon is optionally substituted with a halogen (e.g., F, Cl, Br), methyl, or ethyl;
W^{L3} is a 3-7 membered ring (e.g., 4-6 membered cycloalkyl or heterocycloalkyl) with 0-3 heteroatoms, optionally substituted with a halogen (e.g., F, Cl, Br) or methyl;
Y^{L3} is absent or a C1-C4 alkyl (C₁, C₂, C₃, or C₄ alkyl), wherein one or more C atoms are optionally replaced with O or NH, and wherein: each carbon is optionally substituted with a halogen (e.g., F, Cl, Br) or a linear or branched C1-C4 alkyl; and each NH is optionally substituted with a linear or branched C1-C5 alkyl (e.g., C₁, C₂, C₃, C₄, or C₅ alkyl);
W^{L4} is absent or a 3-7 membered ring (e.g., 4-6 membered cycloalkyl or heterocycloalkyl), each with 0-3 heteroatoms and optionally substituted with halogen (e.g., F, Cl, Br), or methyl;
Y_{L4} is bond, O, or an unsubstituted or substituted linear or branched C1-C3 alkyl, wherein a carbon is optionally replaced with O or NH, and optionally substituted with a halogen (e.g., F, Cl, Br) or methyl; and
the dashed lines indicate attachment points.

In any aspect or embodiment described herein, the until A^{L} of the linker (L) comprises a structure selected from the group consisting of:

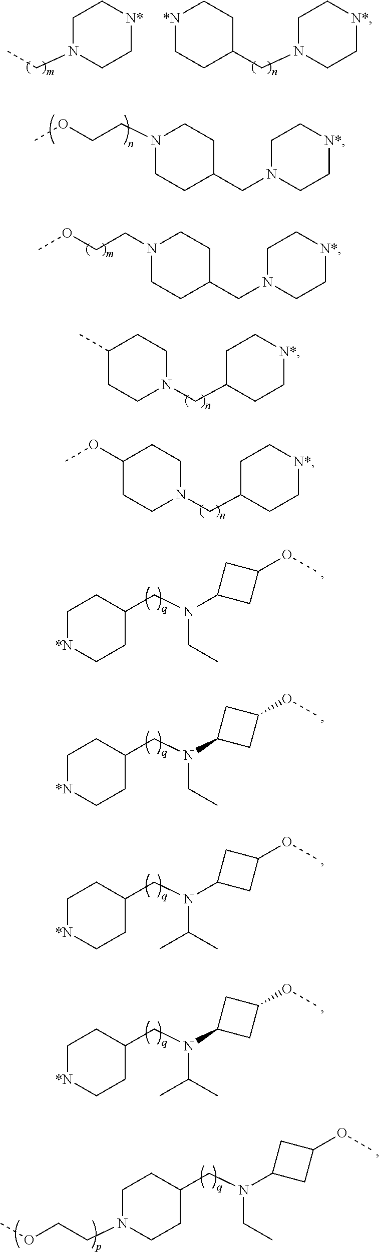

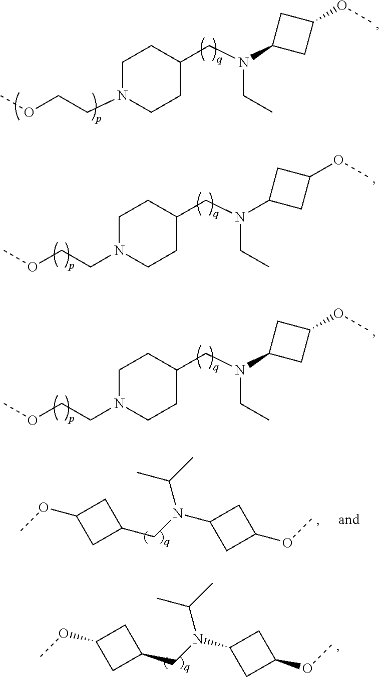

wherein:
m is 1, 2, 3, 4, or 5;
n is 1, 2, or 3;
p is 0 or 1;
q is 1 or 2; [symbol]
   indicates the site that is covalently linked to the CLM or PTM; and
indicates the site that is covalently linked to the CLM or PTM, or is a nitrogen atom that is shared with the CLM or PTM.

In any aspect or embodiment described herein, the unit A^{L} of the linker (L) comprises a structure selected from the group consisting of:

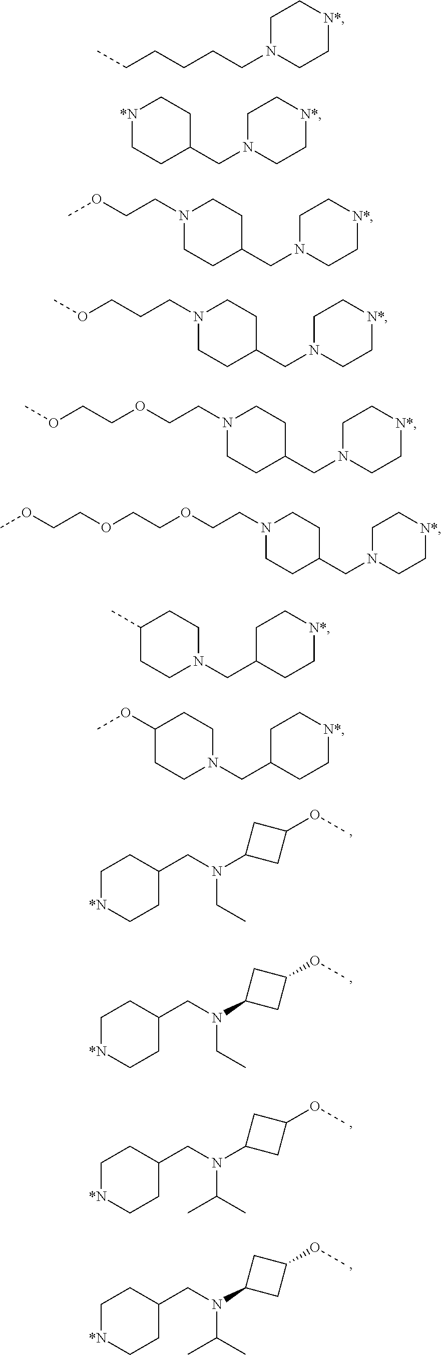

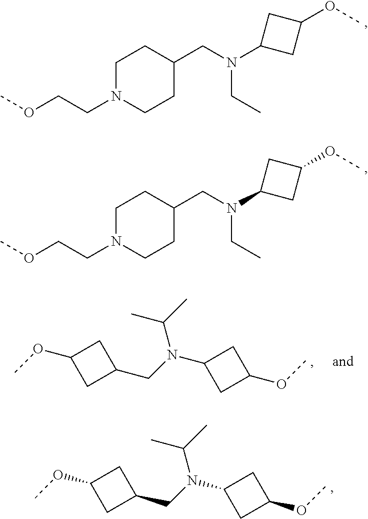

wherein:
[symbol] indicates the site that is covalently linked to the ULM or PTM; and
indicates the site that is covalently linked to the ULM or PTM or is a nitrogen atom that is shared with the ULM or PTM.

Exemplary PTMs

The term “protein target moiety” or PTM is used to describe a small molecule which binds to AR, and can be used to target the PTM for ubiquitination and degradation. The compositions described below exemplify members of AR binding moieties that can be used according to the present disclosure. These binding moieties are linked to the ubiquitin ligase binding moiety preferably through a chemical linking group in order to present the AR protein in proximity to the ubiquitin ligase for ubiquitination and subsequent degradation.

In certain contexts, the term “target protein” is used to refer to the AR protein, which is a target protein to be ubiquitinated and degraded.

The compositions described herein exemplify the use of some of the members of these types of small molecule target protein binding moieties.

In any aspect or embodiment described herein, the PTM is a small molecule that binds AR. For example, in any aspect or embodiment described herein, the PTM is represented by a chemical structure selected from PTM-I, PTM-IIa, PTM-IIb, and PTM-III:

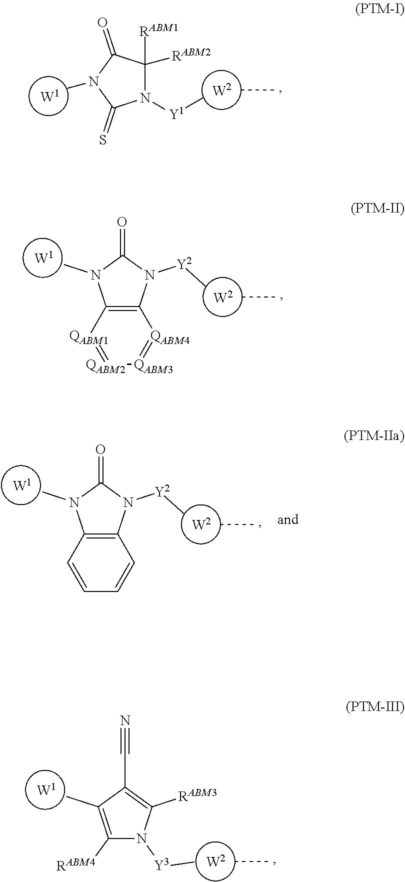

wherein:
W¹ is a 5- or 6-membered aromatic group (e.g., a 5- or 6-membered aryl or heteroaryl) with 0 to 2 heteroatoms (e.g., 0, 1, or 2 nitrogen atoms) substituted with a CN group and optionally substituted with one or more of H, halogen (e.g., F, Cl, or Br), hydroxyl, an unsubstituted or substituted linear or branched C₁_₄ alkyl (e.g., optionally substituted by 1 or more halo), an unsubstituted or substituted linear or branched C₁₋₄ alkoxyl (e.g., optionally substituted by by 1 or more halo), C₁₋₄ haloalkyl (e.g., CF₃);
Y¹ is a bond or a C1-C2 alkyl (e.g., C1 or C2 alkyl), optionally substituted with a methyl, OH, or a halogen;
Y² is a bond or a C1-C5 alkyl (e.g., C₁, C₂, C₃, C₄, or C₅ alkyl), optionally substituted with a methyl, OH, or a halogen;
Y³ is a C1-C2 alkyl, optionally substituted with a methyl, OH, or a halogen; R^{ABM1} and R^{ABM2} are each independently an unsubstituted or substituted C1-C3 alkyl (e.g., an unsubstituted or substituted C1-C2 alkyl or a methyl); or R^{ABM1} and R^{ABM2} together with the carbon to which they are attached form an unsubstituted or substituted C3-C5 membered ring (e.g., an unsubstituted or substituted C3-C5 cycloalkyl, a cyclobutyl group, or an unsubstituted or substituted C3-C5 heterocycloalkyl having 1 or 2 heteroatoms selected from N and O);
R^{ABM3} and R^{ABM4} are each independently a H or an unsubstituted or substituted C1-C3 alkyl (e.g., a methyl or ethyl);
W² is a bond or a 5-7 membered aromatic group with 0 to 2 heteroatoms (e.g., 0, 1, or 2 nitrogen atoms), optionally substituted by 1 or 2 R^{W2};
each of Q_{PTM1}, Q_{PTM2}, Q_{PTM3}, and Q_{PTM4} represent a N or a C substituted with a group selected from H and R;
each R^{W2} is independently: H; OH; NH₂; halogen (e.g., —F or —Cl); linear or branched C₁₋₃ alkyl optionally substituted by 1 or more F; linear or branched C₁₋₃ heteroalkyl optionally substituted by 1 or more F; and OC₁₋₃alkyl optionally substituted by 1 or more —F; and
[symbol] is the linker attachment point.

In any of the aspects or embodiments described herein, W¹ is selected from:

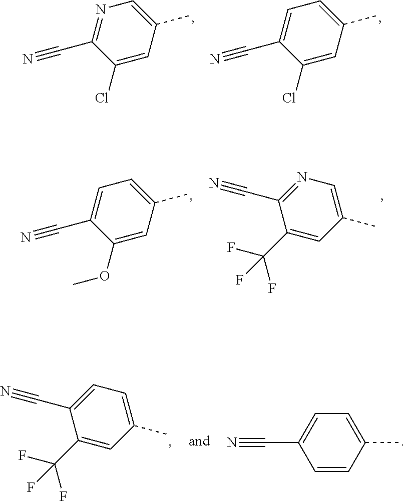

In any aspect or embodiment described herein, W² is a bond or selected from:

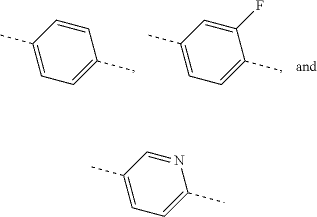

In any aspect or embodiment described herein, R^{ABM1} and R^{ABM2} are each a methyl.

In any aspect or embodiment described herein, R^{ABM1} and R^{ABM2} together with the carbon they are attached form a cyclobutyl group.

In any aspect or embodiment described herein, the PTM is represented by a chemical structure selected from:

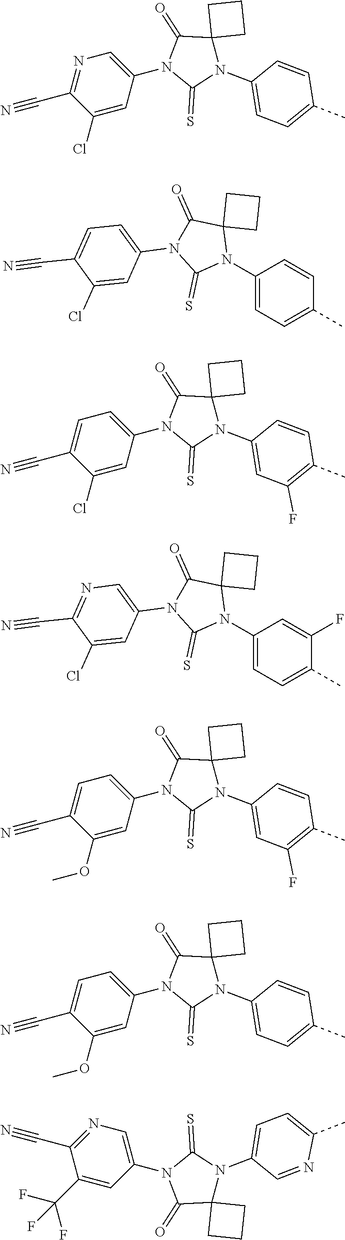

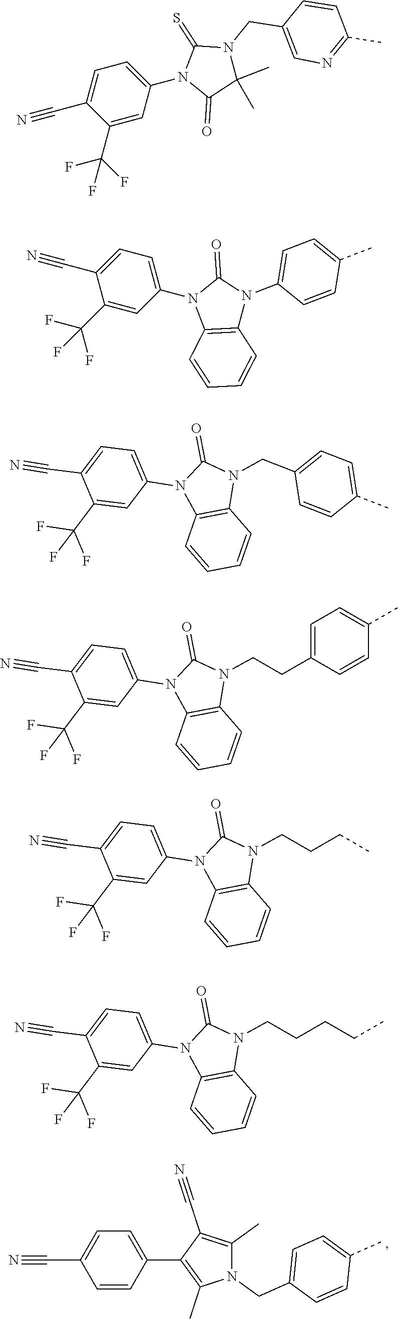

wherein [symbol] of the PTM indicates the point of attachment with a linker group (L).

In any aspect or embodiment described herein, the hetero-bifunctional compound is represented by a chemical structure selected from:

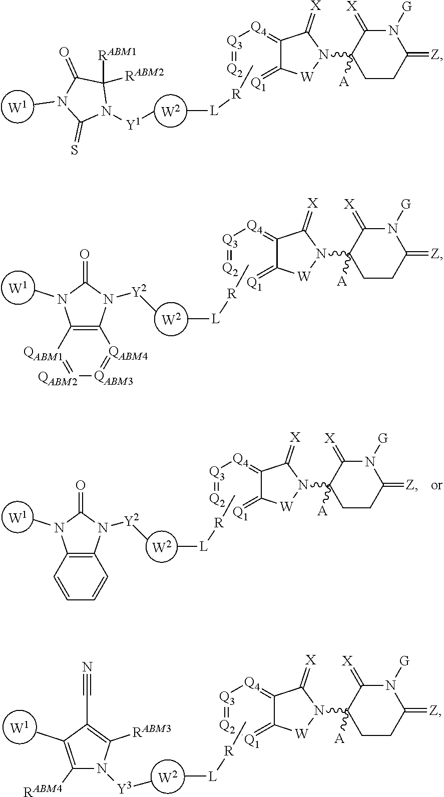

wherein:
R that is covalently linked to L is O, N*, or NH;
N* is a nitrogen atom that is shared with the chemical linking group; and
the other variables (e.g., W¹, R^{ABM1}, R^{ABM2}, Y¹, Y², Y³, W², L, Q₁, Q₂, Q₃, Q₄, W, N, X, A, G, and Z) are as defined in any aspect or embodiment described herein.

In any aspect or embodiment described herein, the hetero-bifunctional compound is represented by a chemical structure selected from:

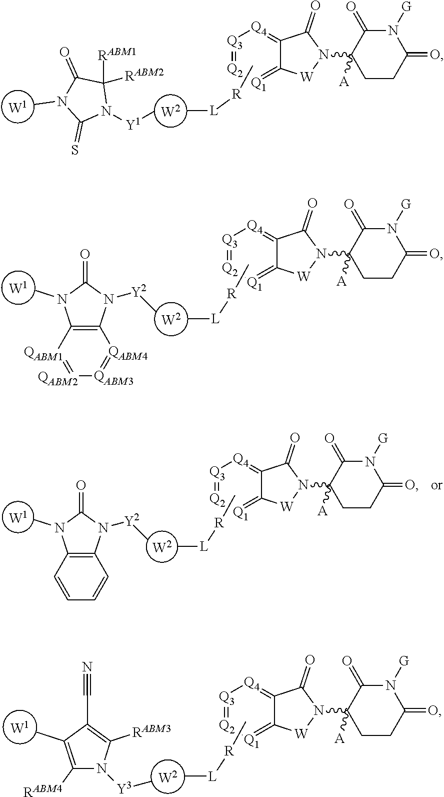

wherein:
R that is covalently linked to L is O, N*, or NH;
N* is a nitrogen atom that is shared with the chemical linking group; and the other variables (e.g., W¹, R^{ABM1}, R^{ABM2} Y¹, Y², Y³, W², L, Q₁, Q₂, Q₃, Q₄, W, N, A, and G) are as defined in any aspect or embodiment described herein.

In any aspect or embodiment described herein, the hetero-bifunctional compound is represented by a chemical structure selected from:

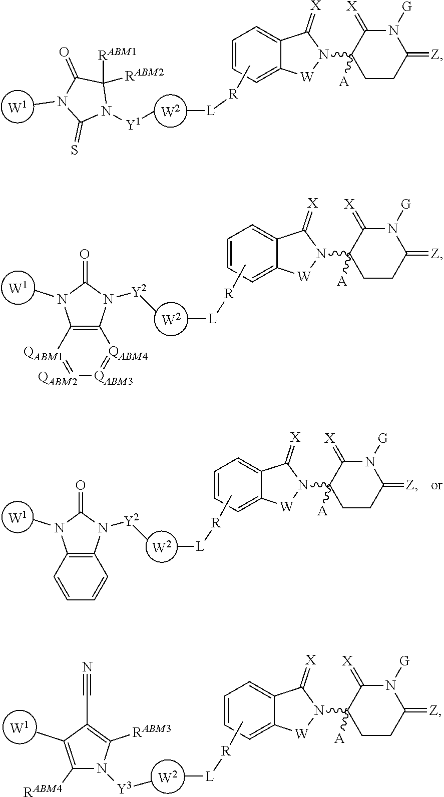

wherein:
R is O, N*, or NH;
N* is a nitrogen atom that is shared with the chemical linking group; and
the other variables (e.g., W¹, R^{ABM1}, R^{ABM2}, Y¹, Y², Y³, W², L, W, N, X, A, G, and Z) are as defined in any aspect or embodiment described herein.

Therapeutic Compositions

The present invention further provides pharmaceutical compositions comprising therapeutically effective amounts of at least one bifunctional compound as described herein, in combination with a pharmaceutically acceptable carrier, additive or excipient.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions effect targeted protein degradation in a patient or subject, for example, an animal such as a human, and can be used for treating or ameliorating disease states or conditions which are modulated by degrading the target protein. In certain embodiments, the therapeutic compositions as described herein may be used to effectuate the degradation of protein for the treatment or amelioration of AR-mediated cancer, such as prostate cancer, Kennedy's disease, or both.

In alternative aspects, the present disclosure relates to a method for treating a disease state or ameliorating one or more symptoms of a disease or condition in a subject in need thereof by degrading the AR protein comprising administering to said patient or subject an effective amount, e.g., a therapeutically effective amount, of at least one compound as described herein, optionally in combination with a pharmaceutically acceptable carrier, additive or excipient, and optionally coadministered with an additional bioactive agent, wherein the composition is effective for treating or ameliorating the disease or disorder or one or more symptoms thereof in the subject. The method according to the present disclosure may be used to treat certain disease states or conditions including cancer and Kennedy's Disease, by virtue of the administration of effective amounts of at least one compound described herein.

The present disclosure further includes pharmaceutical compositions comprising a pharmaceutically acceptable salt, in particular, acid or base addition salts of compounds as described herein. The acids which are used to prepare the pharmaceutically acceptable acid addition salts of the aforementioned compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds according to the present disclosure. The chemical bases that may be used as reagents to prepare pharmaceutically acceptable base salts of the present compounds are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual, intra nasal, intra ocular, intrathecal, and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form or in depot formulation may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient may be combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active pharmaceutical ingredient in a pharmaceutical composition as described herein that may be combined with the carrier materials to produce a single dosage form will vary depending upon the condition of the subject and disease treated, as well as the particular mode of administration. Preferably, the compositions should be formulated to contain between about 0.05 milligram and about 750 milligrams or more, more preferably about 1 milligram to about 600 milligrams, and even more preferably about 10 milligrams to about 500 milligrams of active ingredient, alone or in combination with another compound according to the present disclosure.

It should also be understood that a specific dosage and treatment regimen for any particular patient will depend upon a variety of factors, including the activity and bioavailability of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure depending upon the pharmaceutically acceptable salt, solvate or polymorph, thereof optionally in a pharmaceutically acceptable carrier or diluent, either alone, or in combination with another known therapeutic agent.

The active compound is combined with the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 nanograms per kilograms (ng/kg) to 300 milligrams per kilograms (mg/kg), preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to a dosage form containing less than 1 milligrams (mg), 1 mg to 3000 mg, or 5 mg to 500 mg of active ingredient per unit dosage form. An oral dosage of about 25 mg-250 mg is often convenient.

The active ingredient is preferably administered to achieve peak plasma concentrations of the active compound of about 0.00001-30 millimole (mM), preferably about 0.1-30 micromole (μM). This may be achieved, for example, by the intravenous injection of a solution or formulation of the active ingredient, optionally in saline, or an aqueous medium or administered as a bolus of the active ingredient. Oral administration may also be appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, as described herein among others. In certain preferred aspects of the disclosure, one or more compounds according to the present disclosure are coadministered with another bioactive agent, such as an anti-cancer agent or a wound healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In any aspect or embodiment described herein, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

Therapeutic Methods

In an additional aspect, the description provides therapeutic methods comprising administration of an effective amount of a compound as described herein or salt form thereof, and a pharmaceutically acceptable carrier. The therapeutic methods are useful to effect protein degradation in a patient or subject in need thereof, for example, an animal such as a human, for treating or ameliorating a disease state, condition or related symptom that me be treated through targeted protein degradation.

The terms “treat”, “treating”, and “treatment”, etc., as used herein, refer to any action providing a benefit to a patient for which the present compounds may be administered, including the treatment of any disease state, condition, or symptom which is related to the protein to which the present compounds bind. Disease states or conditions, including cancer, which may be treated using compounds according to the present disclosure are set forth hereinabove.

The description provides therapeutic methods for effectuating the degradation of proteins of interest for the treatment or amelioration of a disease, e.g., cancer. In any aspect or embodiment described herein, the disease is prostate cancer or Kenney's Disease or both. As such, in another aspect, the description provides a method of ubiquitinating/degrading a target protein in a cell. In certain embodiments, the method comprises administering a bifunctional compound of the present disclosure. The control or reduction of specific protein levels in cells of a subject as afforded by the present disclosure provides treatment of a disease state, condition, or symptom. In any aspect or embodiment described herein, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another aspect, the description provides a process for making a molecule that can cause degradation of AR in a cell, comprising the steps of: i. providing a small molecule that binds AR; ii. providing and E3 ubiquitin ligase binding moiety (ULM), preferably a CLM such as thalidomide, pomalidomide, lenalidomide or an analog thereof; and iii. covalently coupling the small molecule of step (i) to the ULM of step (ii) via a chemical linking group (L) to form a compound which binds to both a cereblon E3 ubiquitin ligase and AR protein in the cell, such that the cereblon E3 ubiquitin ligase is in proximity to, and ubiquitinates AR protein bound thereto, such that the ubiquitinated AR is then degraded.

In another aspect, the description provides a method for detecting whether a molecule can trigger degradation of an AR protein in a cell, the method comprising the steps of: (i) providing a molecule for which the ability to trigger degradation of AR protein in a cell is to be detected, said molecule comprising the structure: CLM-L-PTM, wherein CLM is a cereblon E3 ubiquitin ligase binding moiety capable of binding a cereblon E3 ubiquitin ligase in a cell, which CLM is thalidomide, pomalidomide, lenalidomide, or an analog thereof; PTM is a protein targeting moiety, which is a small molecule that binds to AR, said AR having at least one lysine residue available to be ubiquitinated by a cereblon E3 ubiquitin ligase bound to the CLM of the molecule; and L is a chemical linking group that covalently links the CLM to the PTM to form the molecule; (ii) incubating an AR protein-expressing cell in the presence of the molecule of step (i); and (iii) detecting whether the AR protein in the cell has been degraded.

In any of the aspects or embodiments described herein, the small molecule capable of binding AR, is a small molecule as described herein.

In another aspect of said treatment, the present disclosure provides a method of treating a human patient in need of said treatment of a disease state, condition, or symptom causally related to AR expression, over-expression, mutation, misfolding or dysregulation where the degradation of the AR protein will produce a therapeutic effect in the patient, the method comprising administering to the patient an effective amount of a compound according to the present disclosure, optionally in combination with another bioactive agent. The disease state, condition, or symptom may be caused by a microbial agent or other exogenous agent such as a virus, bacteria, fungus, protozoa or other microbe, or may be a disease state, which is caused by expression, overexpression, mutation, misfolding, or dysregulation of the protein, which leads to a disease state, condition, or symptom.

In another aspect, the present disclosure provides a method of treating or ameliorating at least one symptom of a disease or condition in a subject, comprising the steps of: providing a subject identified as having a symptom of a disease or condition causally related to expression, overexpression, mutation, misfolding, or dysregulation of AR protein in the subject, and the symptom of the disease or condition is treated or ameliorated by degrading AR protein in cells of the subject; and administering to the subject therapeutically effective amount of a compound comprising a small molecule of the present disclosure such that the AR protein is degraded, thereby treating or ameliorating at least one symptom of a disease or condition in the subject.

The term “disease state or condition” is used to describe any disease state or condition wherein protein expression overexpression, mutation, misfolding, or dysregulation (e.g., the amount of protein expressed in a patient is elevated) occurs and where degradation of the AR protein to reduce or stabilize the level of AR protein (whether mutated or not) in a patient provides beneficial therapy or relief of symptoms to a patient in need thereof. In certain instances, the disease state, condition, or symptom may be cured.

Disease state, condition, or symptom which may be treated using compounds according to the present disclosure include, for example, cancer, prostate cancer, Kenney's disease. In any aspect or embodiment described herein, the cancer is selected from: squamous-cell carcinoma, basal cell carcinoma, adenocarcinoma, hepatocellular carcinomas, and renal cell carcinomas, bladder cancer, head and neck cancer, kidney cancer, ovary, leukemias, benign and malignant lymphomas, Burkitt's lymphoma, Non-Hodgkin's lymphoma, benign and malignant melanomas, myeloproliferative diseases, sarcoma, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, Schwannomas, bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor or teratocarcinomas. In any aspect or embodiment described herein, the disease to be treated is cancer, e.g., prostate cancer or Kennedy's Disease. In a preferred aspect, the subject is a human.

The term “bioactive agent” is used to describe an agent, other than a compound according to the present disclosure, which is used in combination with a present compound as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer therapeutic agent, which may be combined with a compound according to the present disclosure to cancer. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, an androgen receptor inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(But) 6,Azgly 10] (pyro-Glu-His-Trp-Ser-Tyr-D-Ser(But)-Leu-Arg-Pro-Azgly-NH₂ acetate [C₅₉H₈₄N₁₈Oi₄-(C₂H₄02)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### Examples

### Abbreviations

ACN Acetonitrile

AcOH Acetic acid

DCM Dichloromethane

DMF Dimethylformamide

DMSO Dimethyl Sulfoxide

DIPEA N, N-Diisopropylethylamine

EtOAc/EA Ethyl Acetate

EtOH Ethanol

HATU Hexafluorophosphate Azabenzotriazole Tetramethyl Uronium

HPLC High pressure liquid chromatography

Hz Hertz

KOAc Potassium acetate

LCMS Liquid Chromatography/Mass Spectrometry

MHz Megahertz

NMR Nuclear Magnetic Resonance

MeOH Methanol

MS Mass Spectrometry

PE Petroleum ether

Psi Pound-force per square inch

RT or r.t. Room temperature

TEA Triethylamine

THF Tetrahydrofuran

TFA Trifluoracetic acid

TLC Thin layer chromatography

TMS Trimethylsilyl

General Synthetic Approach

The synthetic realization and optimization of the heterobifunctional molecules as described herein may be approached in a stepwise or modular fashion. For example, identification of compounds that bind to the target protein, i.e., AR can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the chemical linking group previously referred to herein. Where crystallographic or NMR structural data are available, these can be used to focus such a synthetic effort.

In a very analogous way one can identify and optimize ligands for an E3 Ligase.

With PTMs and ULMs (e.g. CLMs) in hand, one skilled in the art can use known synthetic methods for their combination with or without a chemical linking group(s). Chemical linking group(s) can be synthesized with a range of compositions, lengths and flexibility and functionalized such that the PTM and ULM groups can be attached sequentially to distal ends of the linker. Thus, a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

Synthetic Procedures

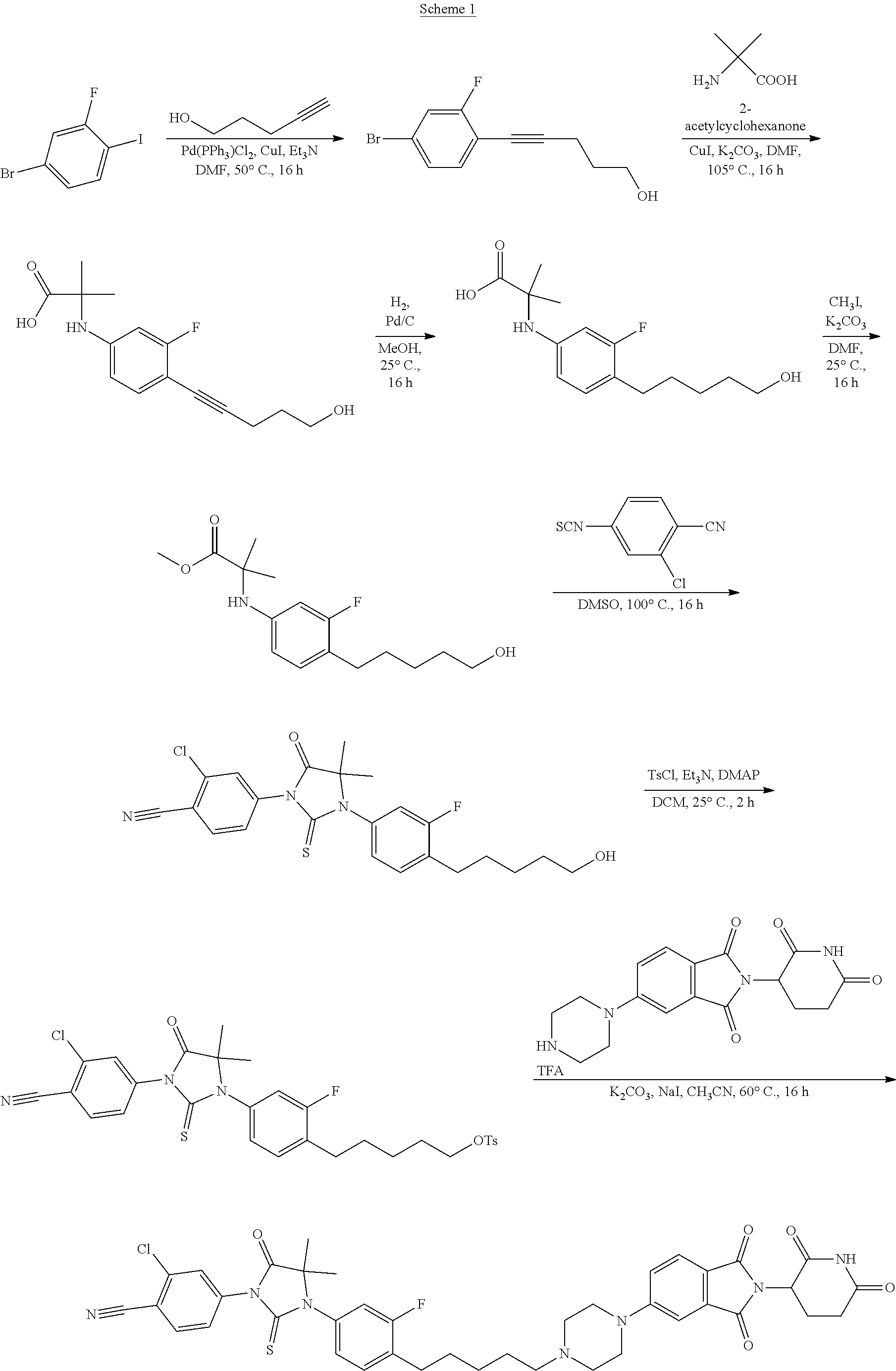

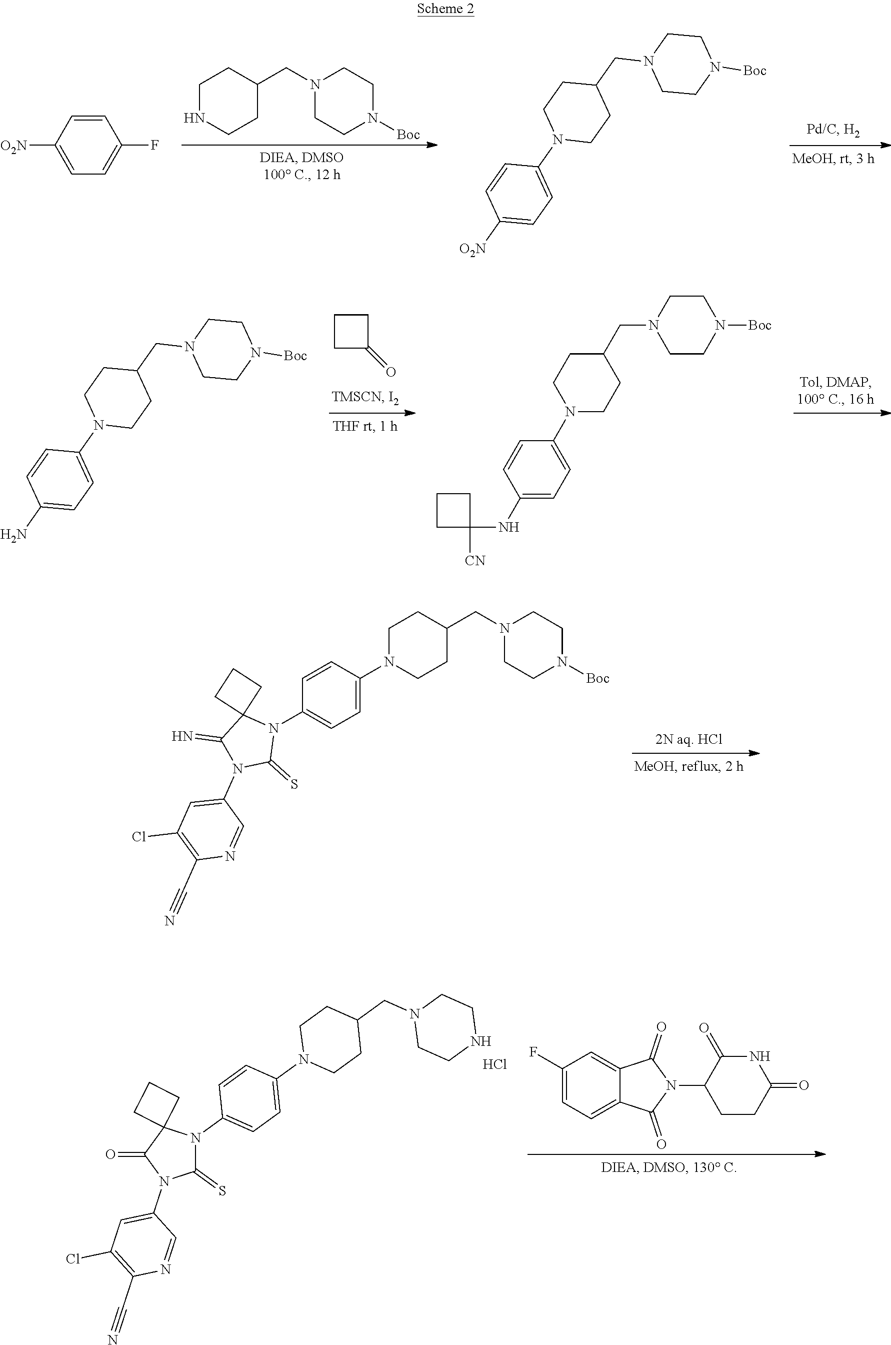

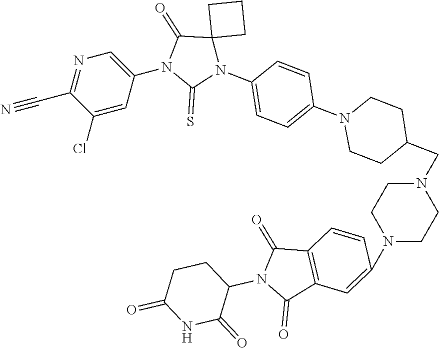 [SMILES: N#Cc1ncc(N2C(=O)C3(CCC3)N(c3ccc(N4CCC(CN5CCN(c6ccc7c(c6)C(=O)N(C6CCC(=O)NC6=O)C7=O)CC5)CC4)cc3)C2=S)cc1Cl]

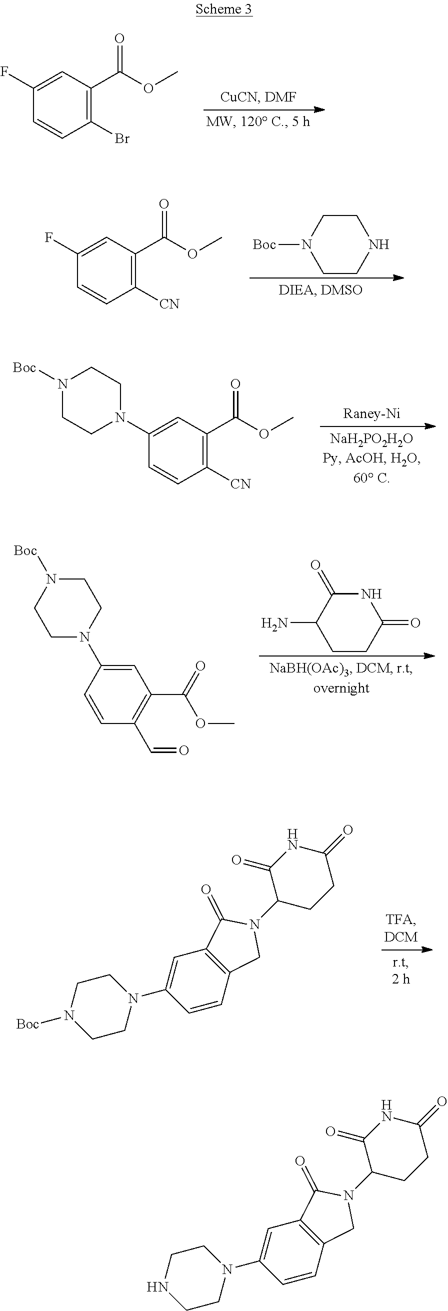

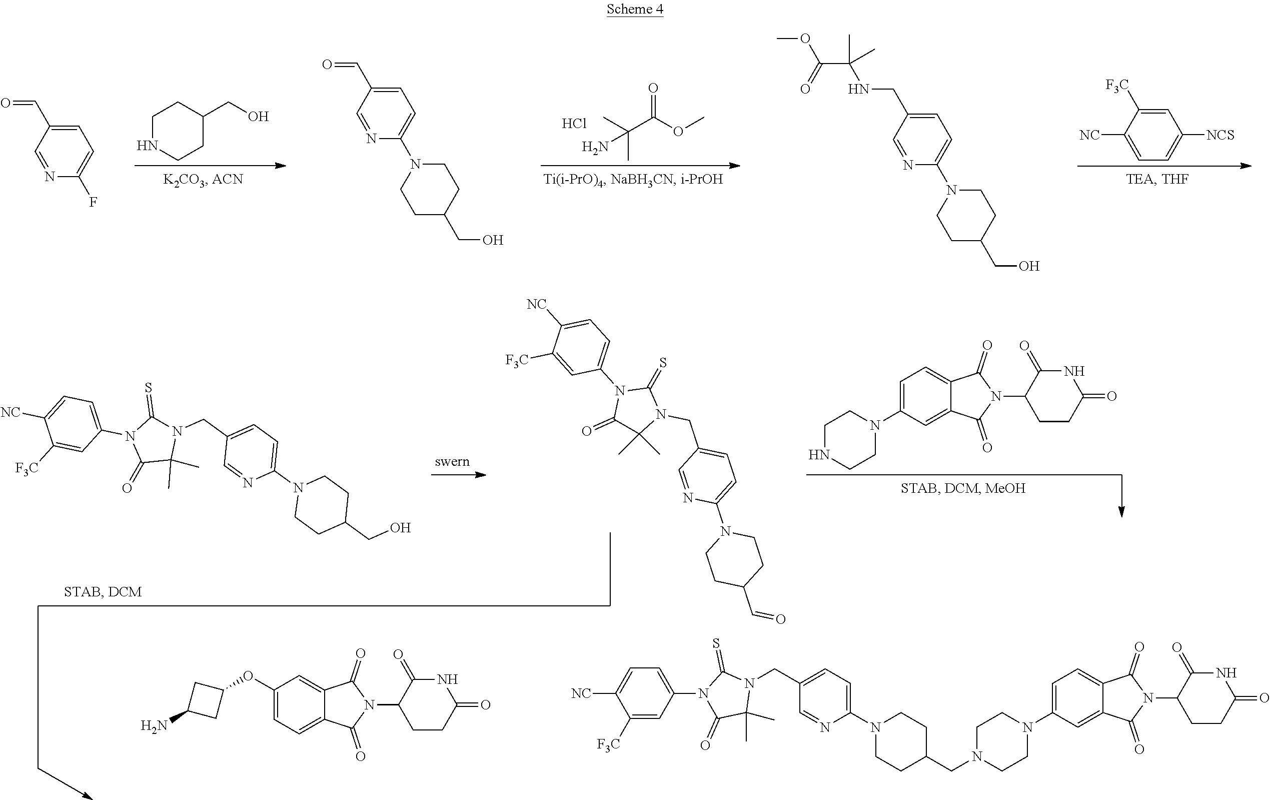

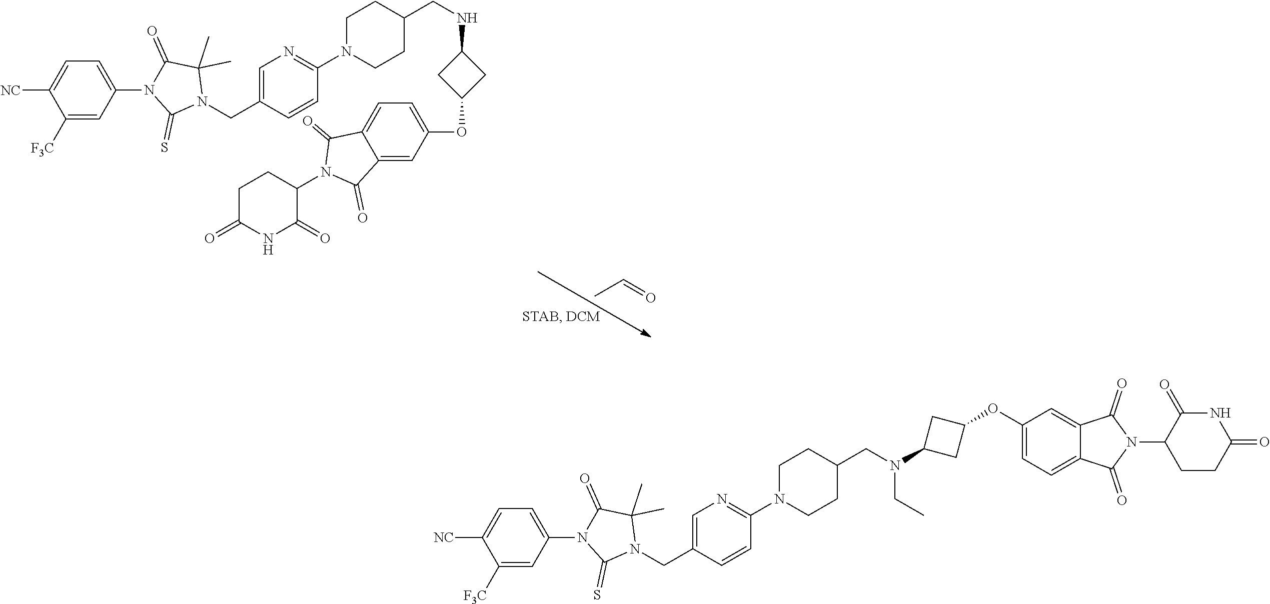

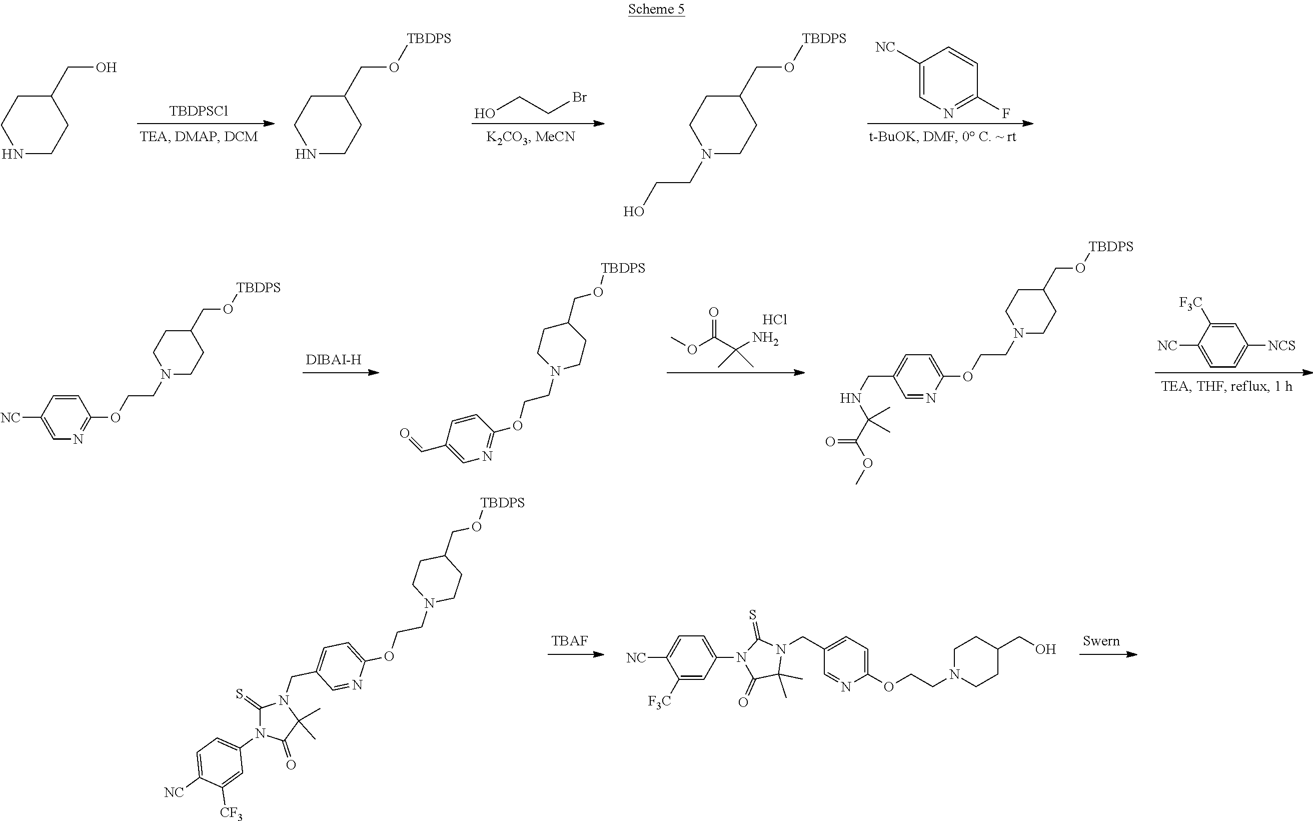

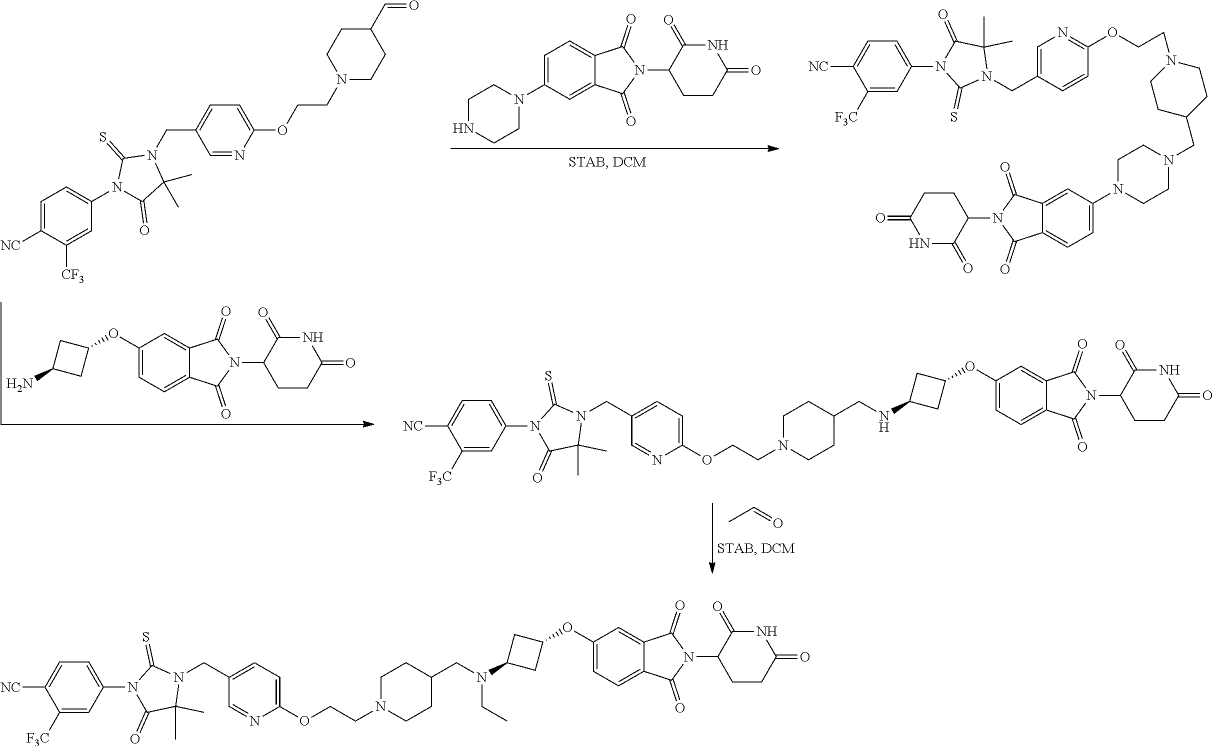

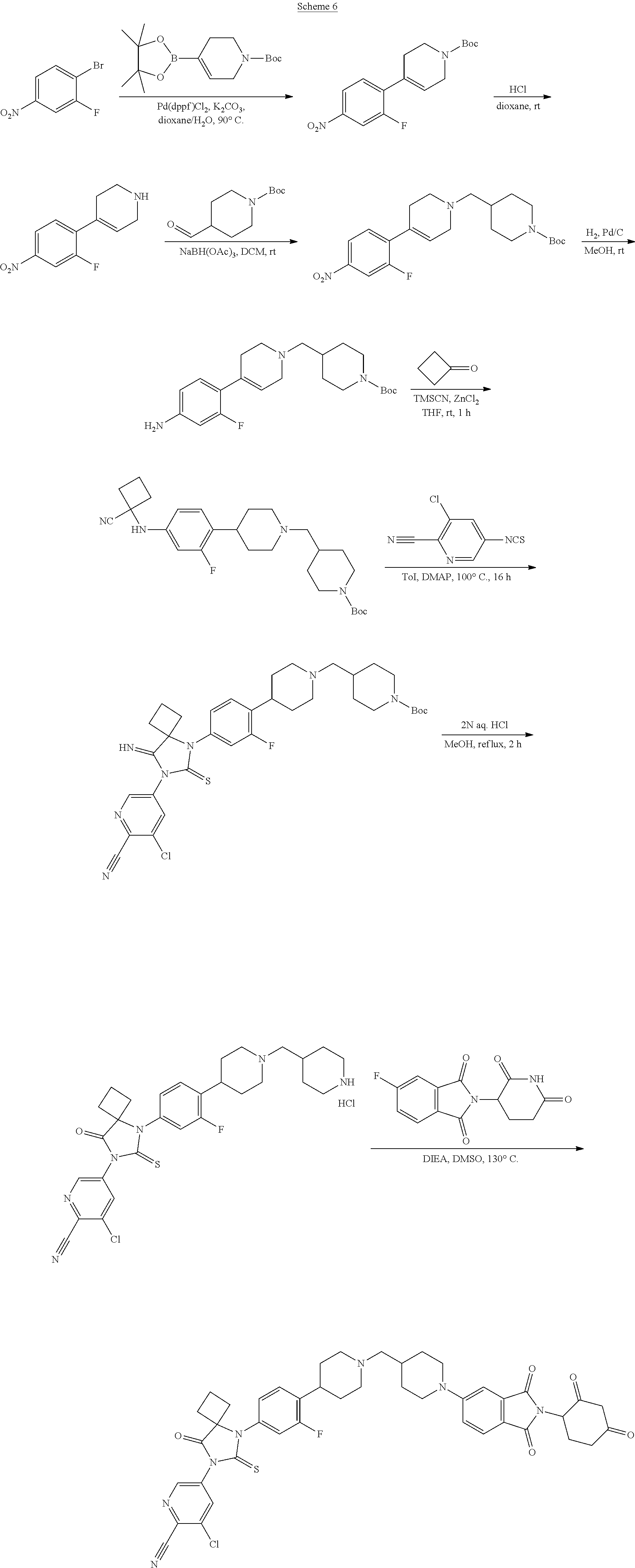

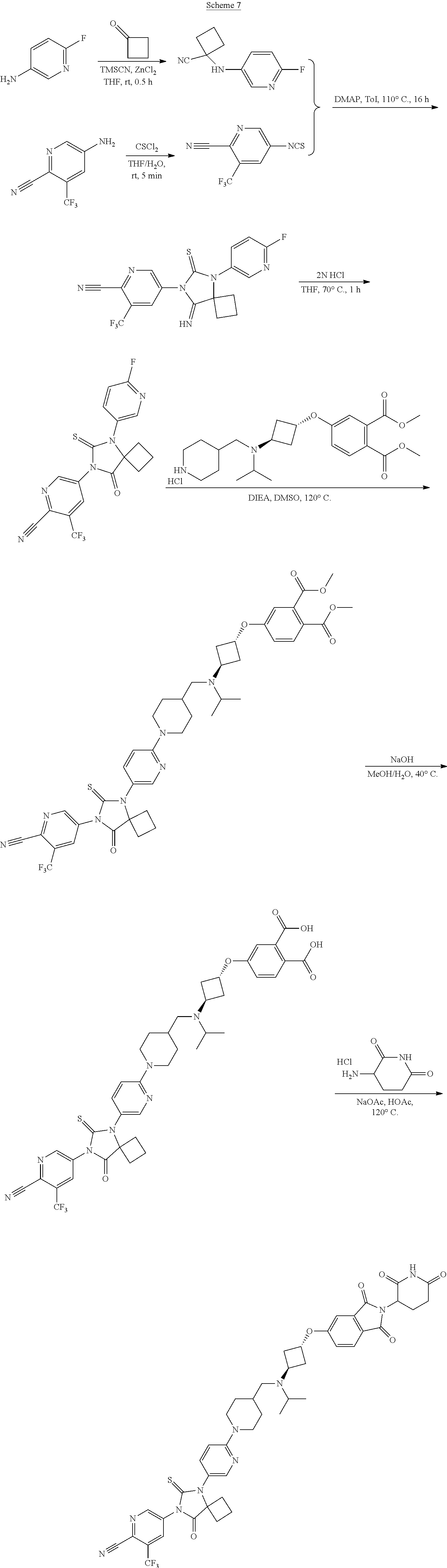

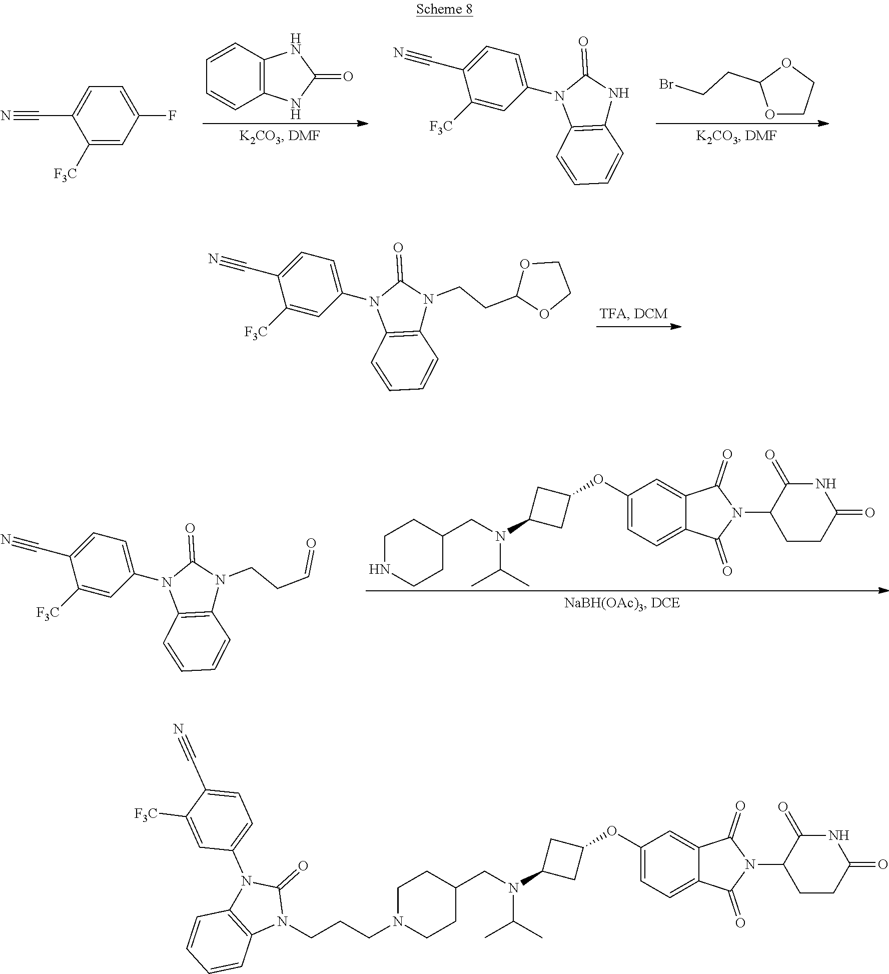

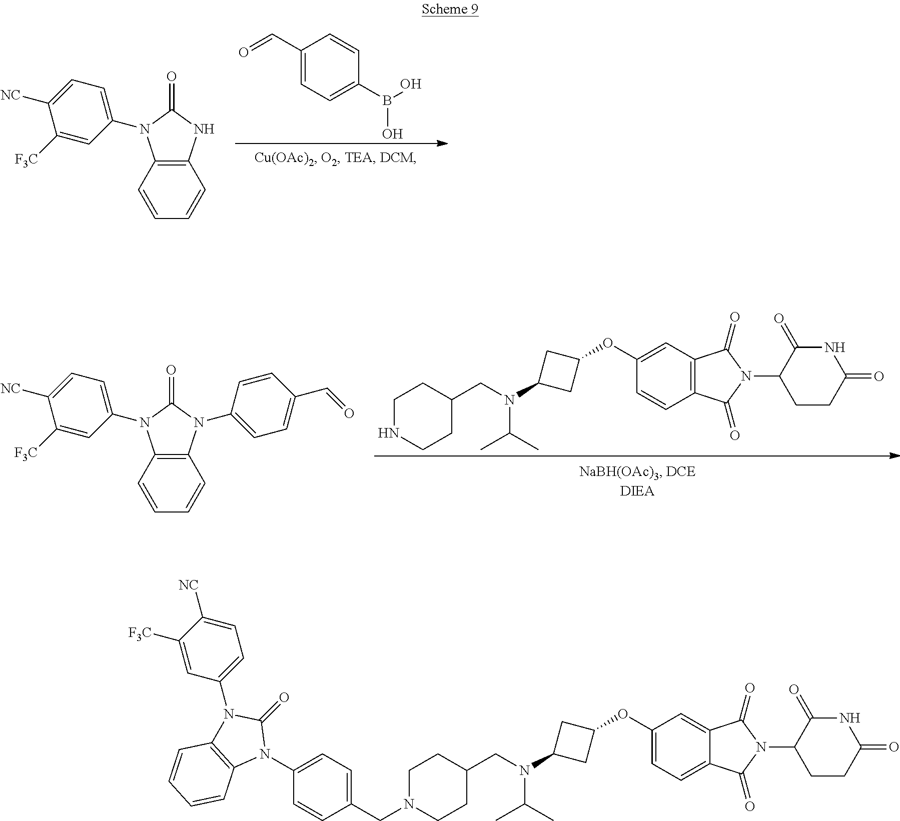

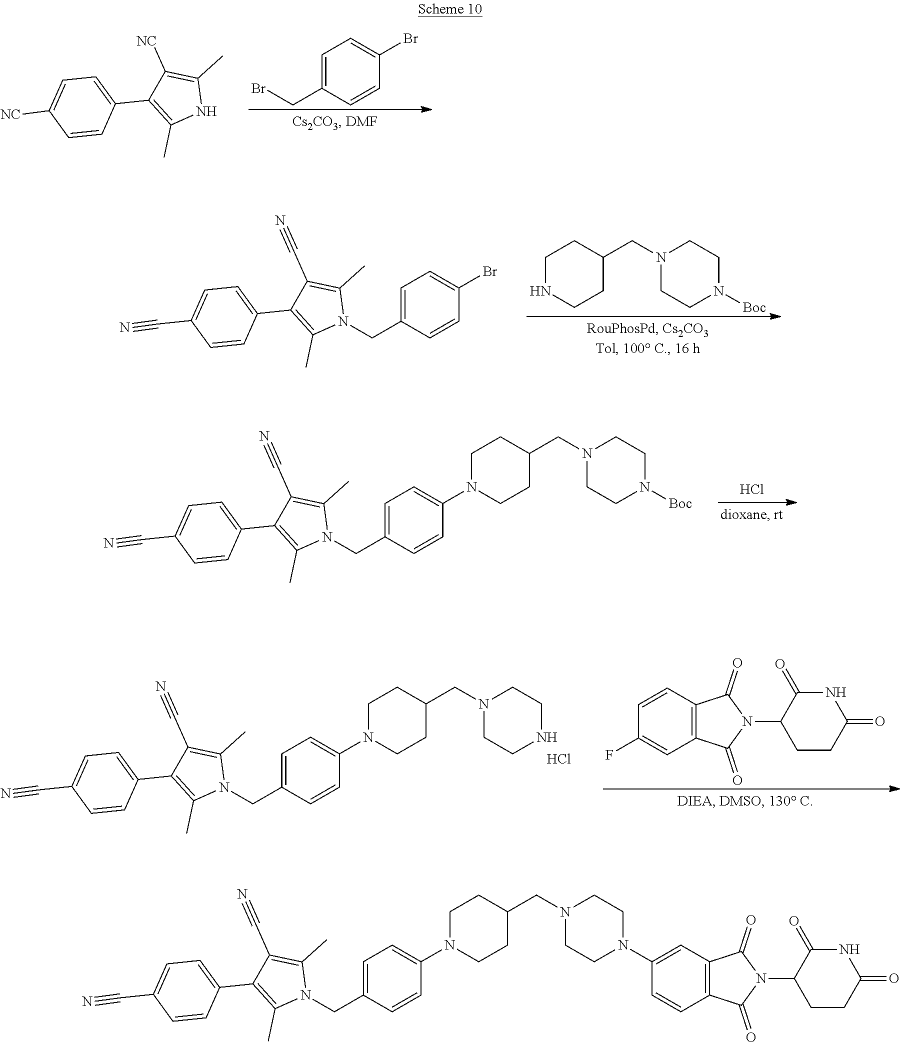

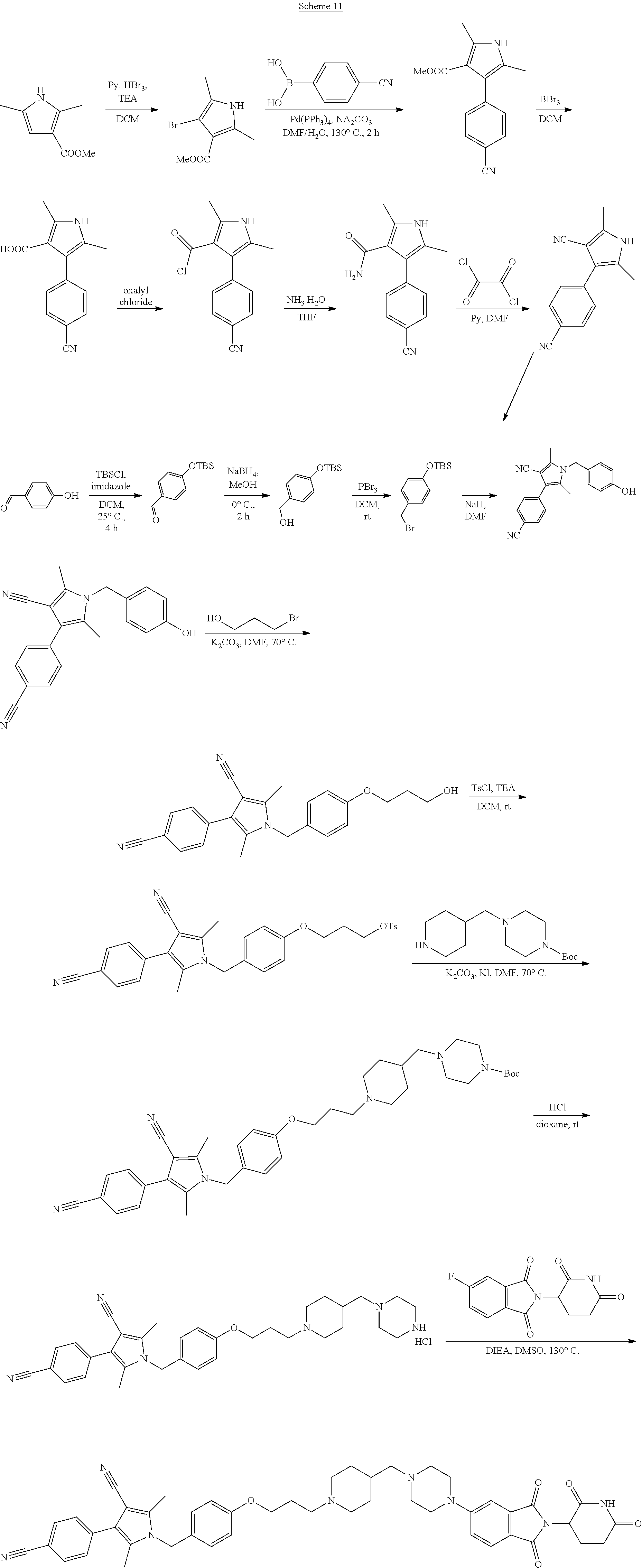

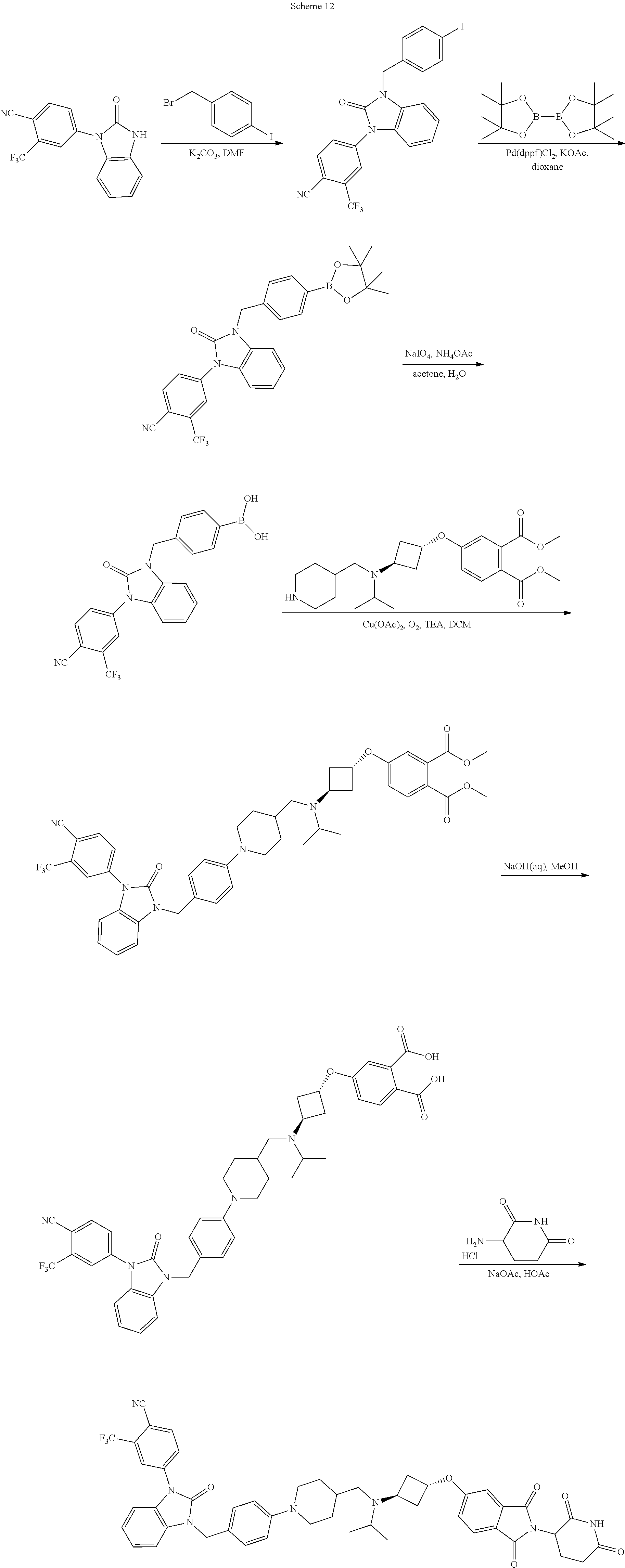

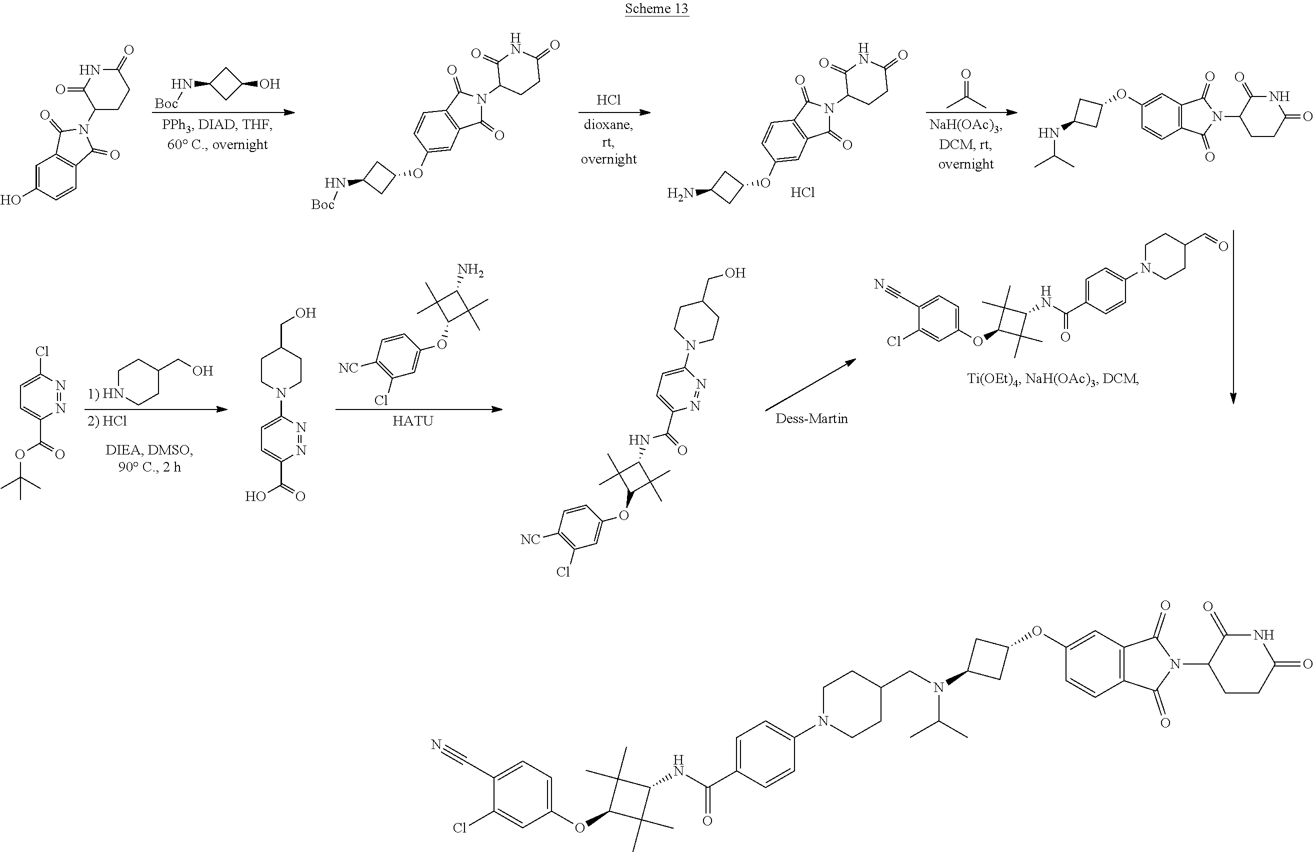

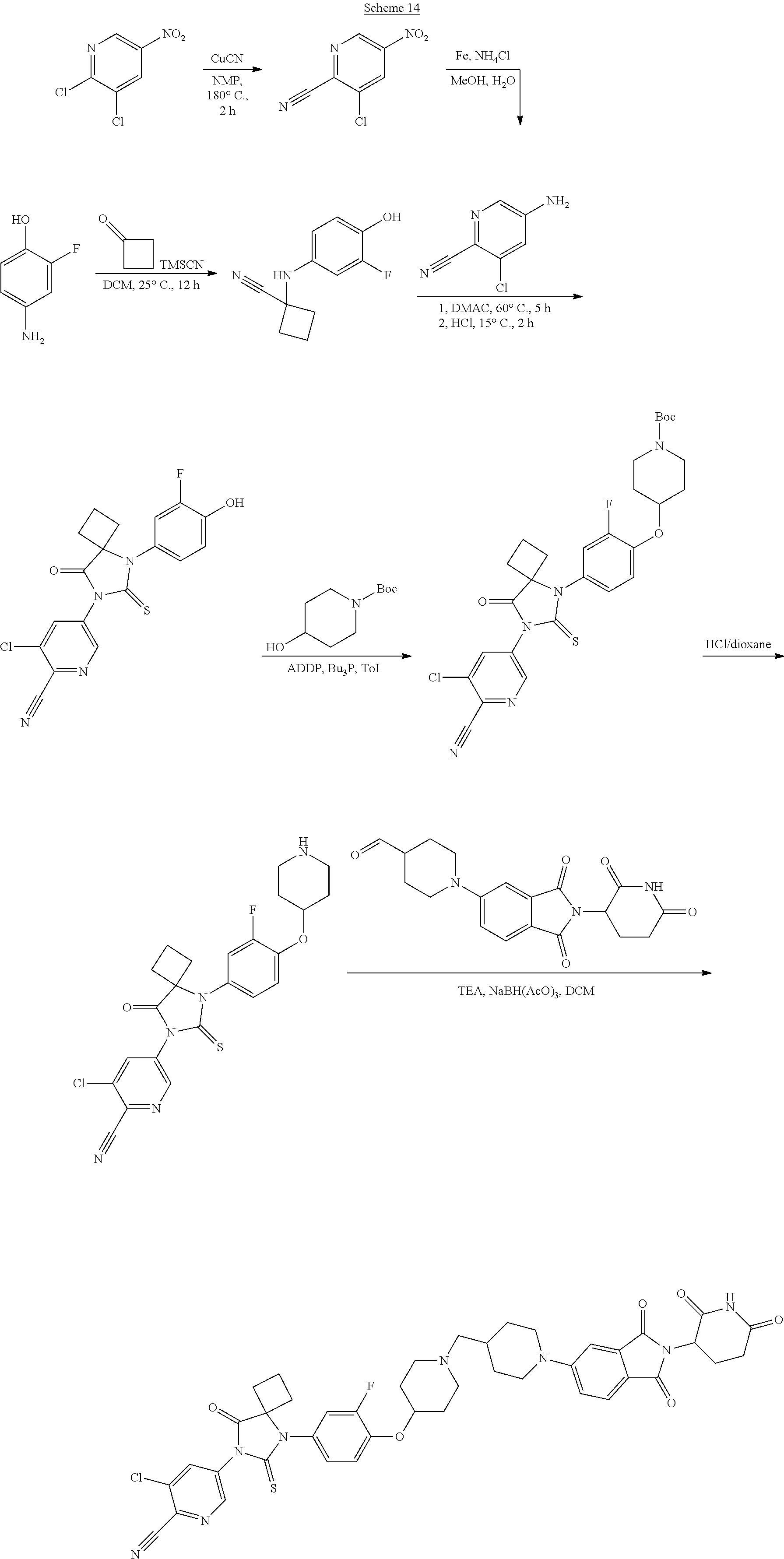

### Exemplary Synthesis of 2-(2,6-dioxopiperidin-3-yl)-5-hydroxyisoindoline-1,3-dione

### Step 1: Preparation of 2-(2,6-dioxopiperidin-3-yl)-5-hydroxyisoindoline-1,3-dione

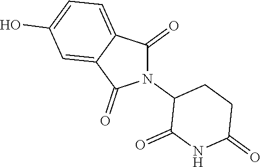 [SMILES: O=C1CCC(N2C(=O)c3ccc(O)cc3C2=O)C(=O)N1]

A solution of 3-aminopiperidine-2,6-dione (4.1 g, 24.7 mmol, 1.50 eq, HCl salt) in acetic acid (45 mL) was charged with sodium acetate (4.1 g, 49.4 mmol, 3.00 eq), then the mixture was stirred at 25° C. for 1 hour. Then 4-hydroxyphthalic acid (3.0 g, 16.5 mmol, 1.00 eq) was added into the mixture and heated to 120° C., stirred for additional 11 hours. The mixture was concentrated and then poured into water (20 mL), and then filtered. The crude product was purified by column chromatography (dichloromethane:methanol=50:1 to 10:1) to afford 2-(2,6-dioxo-3-piperidyl)-5-hydroxy-isoindoline-1,3-dione (3.9 g, 14.3 mmol, 86% yield) as a colorless solid. LC/MS (ESI) m/z: 275 [M+1]⁺; ¹H-NMR (400 MHz, CDCl₃) δ 11.19-10.94 (m, 2H), 7.75 (d, J=8.0 Hz, 1H), 7.20-7.08 (m, 2H), 5.08 (dd, J=5.2, 12.8 Hz, 1H), 3.34 (br s, 1H), 2.95-2.81 (m, 1H), 2.64-2.55 (m, 1H), 2.08-1.98 (m, 1H).

### Exemplary Synthesis of Exemplary Compound 4: 4-(3-((6-(4-((((1r,3r)-3-((2-(2,6-dioxopiperidin-3-yl)-1,3-dioxoisoindolin-5-yl)oxy)cyclobutyl)(ethyl)amino)methyl)piperidin-1-yl)pyridin-3-yl)methyl)-4,4-dimethyl-5-oxo-2-thioxoimidazolidin-1-yl)-2-(trifluoromethyl)benzonitrile

### Step 1: Preparation of 6-(4-(hydroxymethyl)piperidin-1-yl)nicotinaldehyde

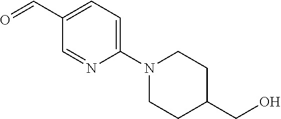 [SMILES: O=Cc1ccc(N2CCC(CO)CC2)nc1]

To a stirred solution of 6-fluoropyridine-3-carbaldehyde (5 g, 39.967 mmol, 1 equiv) and (piperidin-4-yl)methanol (9.21 g, 79.935 mmol, 2 equiv) in MeCN were added K₂CO₃ (11.05 g, 79.935 mmol, 2 equiv) and in portions at room temperature under nitrogen atmosphere. The resulting mixture was stirred overnight at room temperature. The resulting mixture was extracted with CH₂Cl₂ (100 x mL). The combined organic layers were washed with water (2×200 mL), dried over anhydrous Na₂SO₄. After filtration, the filtrate was concentrated under reduced pressure. The residue was purified by silica gel column chromatography, eluted with PE/EtOAc (30˜100%) to afford 6-[4-(hydroxymethyl)piperidin-1-yl]pyridine-3-carbaldehyde (8.5 g, 96.55%) as a light yellow oil. LC/MS (ESI) m/z: 221.27 [M+1]⁺.

### Step 2: Preparation of methyl 2-(((6-(4-(hydroxymethyl)piperidin-1-yl)pyridin-3-yl)methyl)amino)-2-methylpropanoate

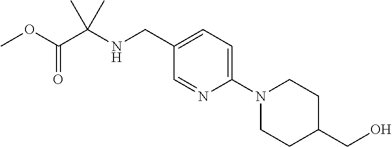 [SMILES: COC(=O)C(C)(C)NCc1ccc(N2CCC(CO)CC2)nc1]

To a stirred solution of 6-[4-(hydroxymethyl)piperidin-1-yl]pyridine-3-carbaldehyde (3 g, 13.620 mmol, 1 equiv) and methyl 2-amino-2-methylpropanoate hydrochloride (6.28 g, 40.859 mmol, 3 equiv) in i-PrOH (80 mL) were added Ti(Oi-Pr)4 (11.61 g, 40.859 mmol, 3 equiv) dropwise. The resulting mixture was stirred for additional 1 hour at room temperature. To the above mixture was added NaBH₃CN (4.28 g, 68.098 mmol, 5 equiv) in portions at 0° C. The resulting mixture was stirred for additional 2 h at room temperature. The residue was purified by silica gel column chromatography, eluted with CH₂Cl₂/MeOH (10:1) to afford methyl 2-[([6-[4-(hydroxymethyl)piperidin-1-yl]pyridin-3-yl]methyl)amino]-2-methylpropanoate (3.1 g, 70.82%) as a yellow oil. LC/MS (ESI) m/z: 322.21 [M+1]⁺.

### Step 3: Preparation of 4-(3-((6-(4-(hydroxymethyl)piperidin-1-yl)pyridin-3-yl)methyl)-4,4-dimethyl-5-oxo-2-thioxoimidazolidin-1-yl)-2-(trifluoromethyl)benzonitrile

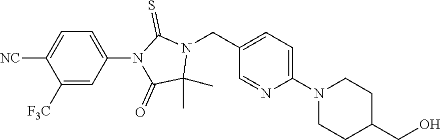 [SMILES: CC1(C)C(=O)N(c2ccc(C#N)c(C(F)(F)F)c2)C(=S)N1Cc1ccc(N2CCC(CO)CC2)nc1]

To a stirred mixture of methyl 2-[([6-[4-(hydroxymethyl)piperidin-1-yl]pyridin-3-yl]methyl)amino]-2-methylpropanoate (500 mg, 1.556 mmol, 1 equiv) and 4-isothiocyanato-2-(trifluoromethyl)benzonitrile (532.46 mg, 2.333 mmol, 1.50 equiv) in THF were added TEA (314.82 mg, 3.111 mmol, 2.00 equiv) dropwise at room temperature under nitrogen atmosphere. The resulting mixture was stirred for 1 hour at 70° C. The resulting mixture was extracted with EtOAc (100 x mL). The combined organic layers were washed with water (2×100 mL), dried over anhydrous Na₂SO₄. After filtration, the filtrate was concentrated under reduced pressure. The residue was purified by Prep-TLC (CH₂Cl₂/MeOH 20:1) to afford 4-[3-([6-[4-(hydroxymethyl)piperidin-1-yl]pyridin-3-yl]methyl)-4,4-dimethyl-5-oxo-2-sulfanylideneimidazolidin-1-yl]-2-(trifluoromethyl)benzonitrile (200 mg, 24.84%) as a light yellow oil. LC/MS (ESI) m/z: 518.18 [M+1]⁺.

### Step 4: Preparation of 4-(3-((6-(4-formylpiperidin-1-yl)pyridin-3-yl)methyl)-4,4-dimethyl-5-oxo-2-thioxoimidazolidin-1-yl)-2-(trifluoromethyl)benzonitrile

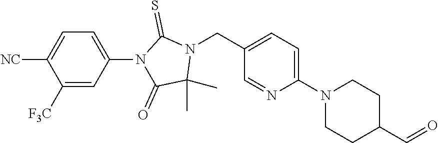 [SMILES: CC1(C)C(=O)N(c2ccc(C#N)c(C(F)(F)F)c2)C(=S)N1Cc1ccc(N2CCC(C=O)CC2)nc1]

Into a 25 mL 2-necked round-bottom flask were added (COCl)₂ (36.79 mg, 0.290 mmol, 1.5 equiv) and DCM (0.2 mL). To the above mixture was added DMSO-DCM (45.29 mg, 0.580 mmol, 3.00 equiv) dropwise at −78° C. The resulting mixture was stirred for additional 30 minutes at −78° C. To the above mixture was added 4-[3-([6-[4-(hydroxymethyl)piperidin-1-yl]pyridin-3-yl]methyl)-4,4-dimethyl-5-oxo-2-sulfanylideneimidazolidin-1-yl]-2-(trifluoromethyl)benzonitrile (100 mg, 0.193 mmol, 1 equiv)-DCM dropwise at −78° C. The resulting mixture was stirred for additional 30 minutes at −78° C. To the above mixture was added TEA (97.75 mg, 0.966 mmol, 5 equiv) dropwise at −78° C. The resulting mixture was stirred for additional 1 hour at room temperature. The residue was purified by Prep-TLC (CH2Cl2/MeOH 20:1) to afford 4-(3-[[6-(4-formylpiperidin-1-yl)pyridin-3-yl]methyl]-4,4-dimethyl-5-oxo-2-sulfanylideneimidazolidin-1-yl)-2-(trifluoromethyl)benzonitrile (50 mg, 50.19%) as a yellow solid. LC/MS (ESI) m/z: 516.16 [M+1]⁺.

### Step 5: Preparation of 4-(3-((6-(4-((((1r,3r)-3-((2-(2,6-dioxopiperidin-3-yl)-1,3-dioxoisoindolin-5-yl)oxy)cyclobutyl)amino)methyl)piperidin-1-yl)pyridin-3-yl)methyl)-4,4-dimethyl-5-oxo-2-thioxoimidazolidin-1-yl)-2-(trifluoromethyl)benzonitrile

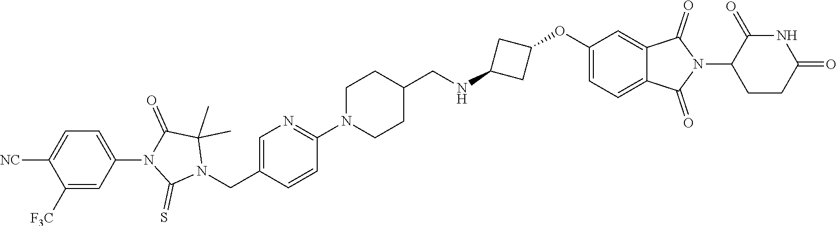 [SMILES: CC1(C)C(=O)N(c2ccc(C#N)c(C(F)(F)F)c2)C(=S)N1Cc1ccc(N2CCC(CN[C@H]3C[C@H](Oc4ccc5c(c4)C(=O)N(C4CCC(=O)NC4=O)C5=O)C3)CC2)nc1]

A solution of 4-(3-[[6-(4-formylpiperidin-1-yl)pyridin-3-yl]methyl]-4,4-dimethyl-5-oxo-2-sulfanylideneimidazolidin-1-yl)-2-(trifluoromethyl)benzonitrile (100 mg, 0.194 mmol, 1 equiv) and 2-(2,6-dioxopiperidin-3-yl)-5-[(1r,3r)-3-aminocyclobutoxy]-2,3-dihydro-1H-isoindole-1,3-dione (79.91 mg, 0.233 mmol, 1.2 equiv) in DCM and MeOH was stirred for 1 h at room temperature under nitrogen atmosphere. To the above mixture was added STAB (123.33 mg, 0.582 mmol, 3 equiv) in portions at 0° C. The resulting mixture was stirred for 2 hours at room temperature under nitrogen atmosphere. The residue was purified by Prep-TLC (CH₂Cl₂/MeOH 1:2) to afford 4-[4,4-dimethyl-5-oxo-3-([6-[4-([[(1r,3r)-3-[[2-(2,6-dioxopiperidin-3-yl)-1,3-dioxo-2,3-dihydro-1H-isoindol-5-yl]oxy]cyclobutyl]amino]methyl)piperidin-1-yl]pyridin-3-yl]methyl)-2-sulfanylideneimidazolidin-1-yl]-2-(trifluoromethyl)benzonitrile (90 mg, 55.05%) as a light yellow solid. LC/MS (ESI) m/z: 843.15 [M+1]⁺.

### Step 6: Preparation of 4-(3-((6-(4-((((1r,3r)-3-((2-(2,6-dioxopiperidin-3-yl)-1,3-dioxoisoindolin-5-yl)oxy)cyclobutyl)(ethyl)amino)methyl)piperidin-1-yl)pyridin-3-yl)methyl)-4,4-dimethyl-5-oxo-2-thioxoimidazolidin-1-yl)-2-(trifluoromethyl)benzonitrile

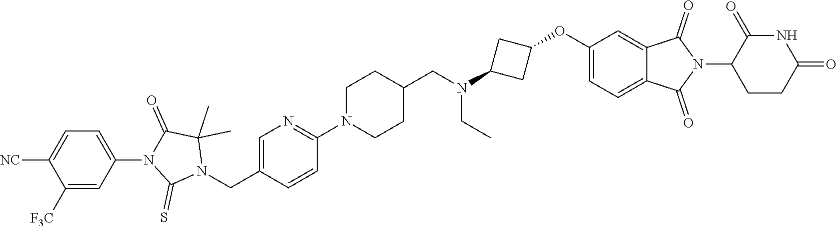 [SMILES: CCN(CC1CCN(c2ccc(CN3C(=S)N(c4ccc(C#N)c(C(F)(F)F)c4)C(=O)C3(C)C)cn2)CC1)[C@H]1C[C@H](Oc2ccc3c(c2)C(=O)N(C2CCC(=O)NC2=O)C3=O)C1]

A solution of 4-[4,4-dimethyl-5-oxo-3-([6-[4-([[(1r,3r)-3-[[2-(2,6-dioxopiperidin-3-yl)-1,3-dioxo-2,3-dihydro-1H-isoindol-5-yl]oxy]cyclobutyl]amino]methyl)piperidin-1-yl]pyridin-3-yl]methyl)-2-sulfanylideneimidazolidin-1-yl]-2-(trifluoromethyl)benzonitrile (90 mg, 0.107 mmol, 1 equiv) and 4-[4,4-dimethyl-5-oxo-3-([6-[4-([[(1r,3r)-3-[[2-(2,6-dioxopiperidin-3-yl)-1,3-dioxo-2,3-dihydro-1H-isoindol-5-yl]oxy]cyclobutyl]amino]methyl)piperidin-1-yl]pyridin-3-yl]methyl)-2-sulfanylideneimidazolidin-1-yl]-2-(trifluoromethyl)benzonitrile (90 mg, 0.107 mmol, 1 equiv) in DCM and MeOH was stirred for 1 hour at room temperature under nitrogen atmosphere. To the above mixture was added STAB (67.89 mg, 0.320 mmol, 3 equiv) in portions at 0° C. The resulting mixture was stirred for 2 hours at 0° C. under nitrogen atmosphere. The crude product was purified by Prep-HPLC. This resulted in 4-[3-([6-[4-([ethyl[(1r,3r)-3-[[2-(2,6-dioxopiperidin-3-yl)-1,3-dioxo-2,3-dihydro-1H-isoindol-5-yl]oxy]cyclobutyl]amino]methyl)piperidin-1-yl]pyridin-3-yl]methyl)-4,4-dimethyl-5-oxo-2-sulfanylideneimidazolidin-1-yl]-2-(trifluoromethyl)benzonitrile (10.7 mg, 11.51%) as a white solid. LC/MS (ESI) m/z: 871.25 [M+1]⁺; ¹H-NMR (400 MHz, CD₃OD) δ 8.24 (d, J=2.4 Hz, 1H), 8.16 (d, J=10.3 Hz, 2H), 7.97 (d, J=7.4 Hz, 1H), 7.85-7.74 (m, 2H), 7.31-7.20 (m, 2H), 6.83 (d, J=8.8 Hz, 1H), 5.12 (dd, J=12.6, 5.5 Hz, 1H), 5.07 (s, 2H), 4.29 (d, J=13.2 Hz, 2H), 3.37 (s, 2H), 2.88 (t, J=13.0 Hz, 3H), 2.77 (d, J=12.1 Hz, 1H), 2.74-2.64 (m, 2H), 2.48 (s, 2H), 2.32 (d, J=6.8 Hz, 4H), 2.20 (d, J=6.9 Hz, 1H), 2.15 (s, 1H), 2.05 (s, 1H), 1.92 (d, J=12.6 Hz, 2H), 1.78 (s, 1H), 1.53 (s, 6H), 1.35 (s, 4H), 1.31 (s, 3H), 1.21 (d, J=12.0 Hz, 3H), 1.04 (t, J=7.1 Hz, 3H), 0.90 (s, 2H), 0.12 (s, 3H).

Protein Level Control

This description also provides methods for the control of protein levels within a cell. The method is based on the use of compounds as described herein such that degradation of the target protein AR in vivo will result in the reducing the amount of the target protein in a biological system, preferably to provide a particular therapeutic benefit.

The following examples are used to assist in describing the present disclosure, but should not be seen as limiting the present disclosure in any way.

In certain embodiments, the description provides the following exemplary AR-degrading bifunctional molecules (compounds of Table 1 or Compounds 1-25), including salts, polymorphs, analogs, derivatives, and deuterated forms thereof.

Assay for Testing AR Degradation Driven by Compounds of the Present Disclosure.

Androgen Receptor ELISA Assay. Compounds were evaluated in the following assay in LNCaP and/or VCaP cells utilizing similar protocols. The protocols used with VCaP cells are described below. The androgen receptor ELISA assay was performed using PathScan AR Sandwich ELISA (Cell Signaling Catalog#12850) according to the following assay steps.

VCaP cells were seeded at 40,000 cells/well at a volume of 100 μL/well in VCaP assay medium [Phenol red free RPMI (Gibco Cat#11835-030); 5% Charcoal Stripped (Dextran treated) FBS (Omega Scientific, Cat#FB-04); 1% penstrep (Life Technologies, Gibco Cat#: 10378-016)] in Corning 3904 plates. The cells were grown for a minimum of 3 days.

First, cells were dosed with compounds diluted in 0.01% DMSO—in a polypropylene plate avoiding the use of outer columns according to the following protocol: (1)(i) 1000× stock plate in DMSO was made; (ii) 20 mM stock diluted 1/6.7 with DMSO (5 μL+28.3 μL DMSO)=3 mM into row H; (iii) serial dilutions in ½ log doses (10 μL of bifunctional compound+20 μL DMSO) was performed from row H towards row B with row A being reserved for DMSO; (iv) 7 doses total (final concentration in this 1000× plate will be 3 mM, 1 mM, 333 μM, 111 μM, etc). (2)(i) A 10× stock plate in media was made; (ii) 2.5 μL of the 1000× stock was trasnferred to a new 10× stock plate (use 12 channel pipet, start at A (DMSO control) work thru H. When 247.5 μL of media was added to this plate, it served as a 10× stock; (iii) made media+1 nM R1881 for making 10× stock plate; (iv) added 247.5 μL of media with 1 nM R1881 to each well of the 10× stock plate, mix.

Then 22 μL of 10× stock was added to cells and incubated for 5 hours. 1× Cell Signaling Cell lysis buffer was made (Catalogue #9803; comes with the kit) to have 50 μL/well, and was kept on ice. Media was aspirated, and 100 μL 1× cell lysis buffer/well was added. The cells were placed on a shaker located in a cold room for 10 minutes and shaken at speed 7. The lysate mixture was mix and 20 μL transferred to 100 μl of Diluent in ELISA plate (0.15 μg/ml-0.075 μg/ml). The lysate-diluent mixture was store at 4° C. overnight on a shaker located in a cold room at speed 5 (gentle swirl).

The lysate-diluent mixture was shaken for 30 minutes at 37° C. The mouse AR antibody, anti-mouse antibody, TMB, and STOP solution were allowed to come to room temperature. The 1×ELISA buffer included in kit was made and loaded in reservoir. Media from the plate was discarded, the ELISA plate was tapped hard on paper towel, and washed 4×200 μl ELISA wash buffer using a plate washer for the first three washes and an eight channel aspirator for the fourth wash to more thoroughly aspirate the solution.

Next, 100 μL/well of mouse AR detection Ab was added; the plate was covered and shaken at 37° C. for 1 hour; media was discarded from the plates, the plates tapped on a paper towel and washed four times with 200 μL ELISA wash buffer with a plate washer for the first three washes and an eight channel aspirator for the fourth wash; 100 μL/well of anti-mouse—HRP conjugated Ab (comes with the kit) was added; the plates wascover and shaken at 37° C. for 30 minutes; the TMB reagent was allowed to come to room temperature; the media from the plate was discarded, the plates tapped on paper towel, and washed four times with 200 μL ELISA wash buffer with a plate washer for the first three washes and an eight channel aspirator for the fourth wash; plates were tapped on paper towl; 100 μL TMB was added to each well and the plate shaken for 2 minutes—while watching color. STOP solution (100 μL) was added when light blue color developed. The plates were shake and read at 450 nM.

Progression of prostate cancer in patients treated with anti-androgen therapy usually involves one of several mechanisms of enhanced Androgen Receptor (AR) signaling, including increased intratumoral androgen synthesis, increased AR expression and AR mutations. Bifunctional molecules described herein simultaneously bind a target of choice and an E3 ligase, cause ubiquitination via induced proximity and degradation of the targeted, pathological protein. As opposed to traditional target inhibition, which is a competitive process, degradation is a progressive process. As such, it is less susceptible to increases in endogenous ligand, target expression, or mutations in the target. Thus this technology seems ideal for addressing the mechanisms of AR resistance in patients with prostate cancer.

Data was analyzed and plotted using GraphPad Prism software. Exemplary compounds of of Table 1 were assayed and the data shown below in Table 2. DC50 (μM) categories (degradation of AR ELISA in LNCaP and/or VCaP cells) of Table 2 are as follows: A<1 nM; 1≤B<10 nM; 10≤C<100 nM; D≥100 nM. Dmax (%) categories of Table 2 are as follows: A≥70; 50≤B<70; C<50.

TABLE 1
Exemplary heterobifunctional compounds of the present disclosure.

| Comp | | | |
|---|---|---|---|
| No. | Structure | Name | Scheme |
| 1 | 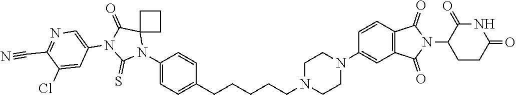 [SMILES: N#Cc1ncc(N2C(=O)C3(CCC3)N(c3ccc(CCCCCN4CCN(c5ccc6c(c5)C(=O)N(C5CCC(=O)NC5=O)C6=O)CC4)cc3)C2=S)cc1Cl] | 3-chloro-5-(5-(4- (5-(4-(2-(2,6- dioxopiperidin-3- yl)-1,3- dioxoisoindolin-5- yl)piperazin-1- | 1 |
| | | yl)pentyl)phenyl)- | |
| | | 8-oxo-6-thioxo- | |
| | | 5,7- | |
| | | diazaspiro[3.4] | |
| | | octan-7- | |
| | | yl)picolinonitrile | |
| 2 | 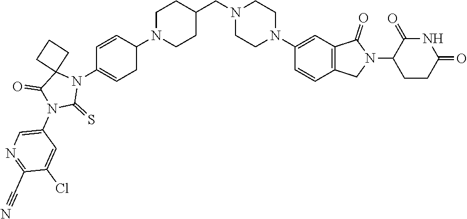 [SMILES: N#Cc1ncc(N2C(=O)C3(CCC3)N(C3=CCC(N4CCC(CN5CCN(c6ccc7c(c6)C(=O)N(C6CCC(=O)NC6=O)C7)CC5)CC4)C=C3)C2=S)cc1Cl] | 3-chloro-5-(5-(4- (4-((4-(2-(2,6- dioxopiperidin-3- yl)-3- oxoisoindolin-5- yl)piperazin-1- yl)methyl)piperidin- 1-yl)phenyl)-8- oxo-6-thioxo-5,7- diazaspiro[3.4] octan-7- yl)picolinonitrile | 2 and 3 |
| 3 | 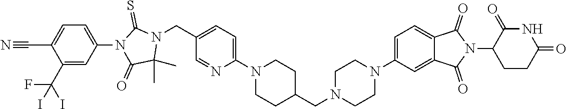 [SMILES: CC1(C)C(=O)N(c2ccc(C#N)c(C(F)(I)I)c2)C(=S)N1Cc1ccc(N2CCC(CN3CCN(c4ccc5c(c4)C(=O)N(C4CCC(=O)NC4=O)C5=O)CC3)CC2)nc1] | 4-(3-((6-(4-((4-(2- (2,6- dioxopiperidin-3- yl)-1,3- dioxoisoindolin-5- yl)piperazin-1- yl)methyl)piperidin- | 4 |
| | | 1-yl)pyridin-3- | |
| | | yl)methyl)-4,4- | |
| | | dimethyl-5-oxo-2- | |
| | | thioxoimidazolidin- | |
| | | 1-yl)-2- | |
| | | (trifluoromethyl) | |
| | | benzonitrile | |
| 4 | 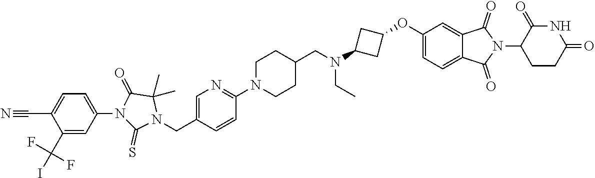 [SMILES: CCN(CC1CCN(c2ccc(CN3C(=S)N(c4ccc(C#N)c(C(F)(F)I)c4)C(=O)C3(C)C)cn2)CC1)[C@H]1C[C@H](Oc2ccc3c(c2)C(=O)N(C2CCC(=O)NC2=O)C3=O)C1] | 4-(3-((6-(4- ((((1r,3r)-3-((2- (2,6- dioxopiperidin-3- yl)-1,3- dioxoisoindolin-5- yl)oxy)cyclobutyl) (ethyl)amino) methyl)piperidin-1- yl)pyridin-3- yl)methyl)-4,4- | 4 |
| | | dimethyl-5-oxo-2- | |
| | | thioxoimidazolidin- | |
| | | 1-yl)-2- | |
| | | (trifluoromethyl) | |
| | | benzonitrile | |
| 5 | 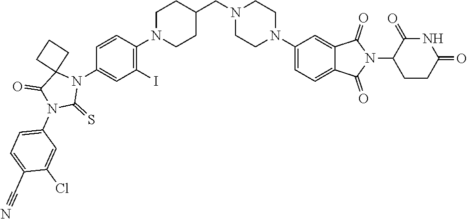 [SMILES: N#Cc1ccc(N2C(=O)C3(CCC3)N(c3ccc(N4CCC(CN5CCN(c6ccc7c(c6)C(=O)N(C6CCC(=O)NC6=O)C7=O)CC5)CC4)c(I)c3)C2=S)cc1Cl] | 2-chloro-4-(5-(4- (4-((4-(2-(2,6- dioxopiperidin-3- yl)-1,3- dioxoisoindolin-5- yl)piperazin-1- yl)methyl)piperidin- 1-yl)-3- fluorophenyl)-8- oxo-6-thioxo-5,7- diazaspiro[3.4] octan-7- yl)benzonitrile | 2 |
| 6 | 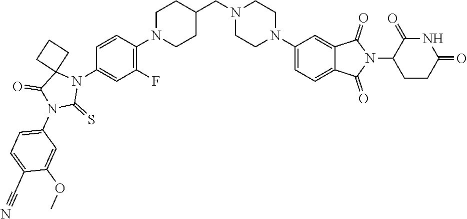 [SMILES: COc1cc(N2C(=O)C3(CCC3)N(c3ccc(N4CCC(CN5CCN(c6ccc7c(c6)C(=O)N(C6CCC(=O)NC6=O)C7=O)CC5)CC4)c(F)c3)C2=S)ccc1C#N] | 4-(5-(4-(4-((4-(2- (2,6- dioxopiperidin-3- yl)-1,3- dioxoisoindolin-5- yl)piperazin-1- yl)methyl)piperidin- 1-yl)-3- fluorophenyl)-8- oxo-6-thioxo-5,7- diazaspiro[3.4] octan-7-yl)-2- methoxybenzonitrile | 2 |
| 7 | 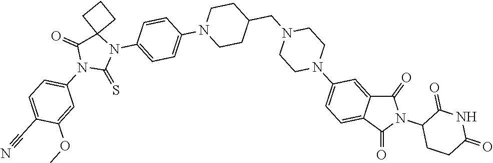 [SMILES: COc1cc(N2C(=O)C3(CCC3)N(c3ccc(N4CCC(CN5CCN(c6ccc7c(c6)C(=O)N(C6CCC(=O)NC6=O)C7=O)CC5)CC4)cc3)C2=S)ccc1C#N] | 4-(5-(4-(4-((4-(2- (2,6- dioxopiperidin-3- yl)-1,3- dioxoisoindolin-5- yl)piperazin-1- yl)methyl)piperidin- 1-yl)phenyl)-8- oxo-6-thioxo-5,7- diazaspiro[3.4] | 2 |
| | | octan-7-yl)-2- | |
| | | methoxybenzonitrile | |
| 8 | 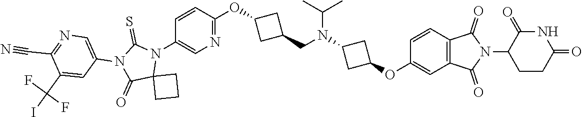 [SMILES: CC(C)N(C[C@H]1C[C@H](Oc2ccc(N3C(=S)N(c4cnc(C#N)c(C(F)(F)I)c4)C(=O)C34CCC4)cn2)C1)[C@H]1C[C@H](Oc2ccc3c(c2)C(=O)N(C2CCC(=O)NC2=O)C3=O)C1] | 5-(5-(6-((1R,3r)- 3-((((1r,3R)-3-((2- (2,6- dioxopiperidin-3- yl)-1,3- dioxoisoindolin-5- yl)oxy)cyclobutyl) | 5 |
| | | (isopropyl)amino) | |
| | | methyl)cyclobutoxy) | |
| | | pyridin-3-yl)- | |
| | | 8-oxo-6-thioxo- | |
| | | 5,7- | |
| | | diazaspiro[3.4] | |
| | | octan-7-yl)-3- | |
| | | (trifluoromethyl) | |
| | | picolinonitrile | |
| 9 | 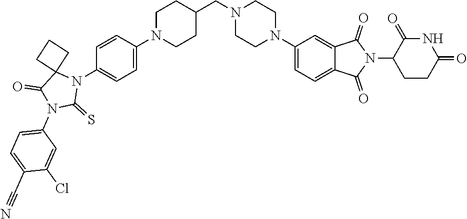 [SMILES: N#Cc1ccc(N2C(=O)C3(CCC3)N(c3ccc(N4CCC(CN5CCN(c6ccc7c(c6)C(=O)N(C6CCC(=O)NC6=O)C7=O)CC5)CC4)cc3)C2=S)cc1Cl] | 2-chloro-4-(5-(4- (4-((4-(2-(2,6- dioxopiperidin-3- yl)-1,3- dioxoisoindolin-5- yl)piperazin-1- yl)methyl)piperidin- 1-yl)phenyl)-8- oxo-6-thioxo-5,7- diazaspiro[3.4] octan-7- yl)benzonitrile | 2 |
| 10 | 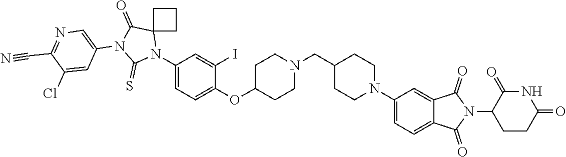 [SMILES: N#Cc1ncc(N2C(=O)C3(CCC3)N(c3ccc(OC4CCN(CC5CCN(c6ccc7c(c6)C(=O)N(C6CCC(=O)NC6=O)C7=O)CC5)CC4)c(I)c3)C2=S)cc1Cl] | 3-chloro-5-(5-(4- ((1-((1-(2-(2,6- dioxopiperidin-3- yl)-1,3- dioxoisoindolin-5- yl)piperidin-4- yl)methyl)piperidin- 4-yl)oxy)-3- fluorophenyl)-8- oxo-6-thioxo-5,7- | 14 |
| | | diazaspiro[3.4] | |
| | | octan-7- | |
| | | yl)picolinonitrile | |
| 11 | 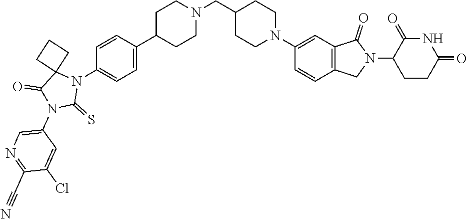 [SMILES: N#Cc1ncc(N2C(=O)C3(CCC3)N(c3ccc(C4CCN(CC5CCN(c6ccc7c(c6)C(=O)N(C6CCC(=O)NC6=O)C7)CC5)CC4)cc3)C2=S)cc1Cl] | 3-chloro-5-(5-(4- (1-((1-(2-(2,6- dioxopiperidin-3- yl)-3- oxoisoindolin-4- yl)methyl)piperidin- 4-yl)phenyl)-8- oxo-6-thioxo-5,7- diazaspiro[3.4] octan-7- yl)picolinonitrile | 6 |
| 12 | 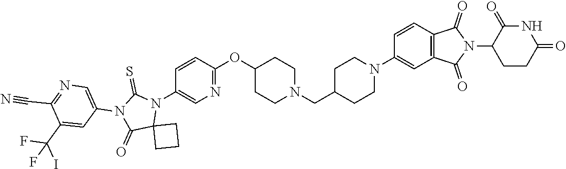 [SMILES: N#Cc1ncc(N2C(=O)C3(CCC3)N(c3ccc(OC4CCN(CC5CCN(c6ccc7c(c6)C(=O)N(C6CCC(=O)NC6=O)C7=O)CC5)CC4)nc3)C2=S)cc1C(F)(F)I] | 5-(5-(6-((1-((1-(2- (2,6- dioxopiperidin-3- yl)-1,3- dioxoisoindolin-5- yl)piperidin-4- yl)methyl) piperidin-4- yl)oxy)pyridin-3- yl)-8-oxo-6- thioxo-5,7- | 14 |
| | | diazaspiro[3.4] | |
| | | octan-7-yl)-3- | |
| | | (trifluoromethyl) | |
| | | picolinonitrile | |
| 13 | 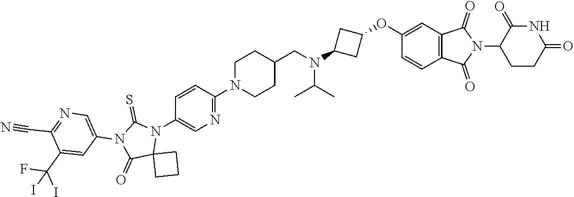 [SMILES: CC(C)N(CC1CCN(c2ccc(N3C(=S)N(c4cnc(C#N)c(C(F)(I)I)c4)C(=O)C34CCC4)cn2)CC1)[C@H]1C[C@H](Oc2ccc3c(c2)C(=O)N(C2CCC(=O)NC2=O)C3=O)C1] | 5-(5-(6-(4- ((((1r,3r)-3-((2- (2,6- dioxopiperidin-3- yl)-1,3- dioxoisoindolin-5- yl)oxy)cyclobutyl) (isopropyl)amino) methyl)piperidin- 1-yl)pyridin-3-yl)- 8-oxo-6-thioxo- 5,7- diazaspiro[3.4] | 7 |
| | | octan-7-yl)-3- | |
| | | (trifluoromethyl) | |
| | | picolinonitrile | |
| 14 | 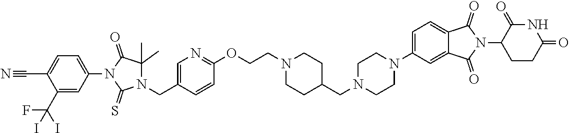 [SMILES: CC1(C)C(=O)N(c2ccc(C#N)c(C(F)(I)I)c2)C(=S)N1Cc1ccc(OCCN2CCC(CN3CCN(c4ccc5c(c4)C(=O)N(C4CCC(=O)NC4=O)C5=O)CC3)CC2)nc1] | 4-(3-((6-(2-(4-((4- (2-(2,6- dioxopiperidin-3- yl)-1,3- dioxoisoindolin-5- yl)piperazin-1- yl)methyl) piperidin-1- | 5 |
| | | yl)ethoxy)pyridin- | |
| | | 3-yl)methyl)-4,4- | |
| | | dimethyl-5-oxo-2- | |
| | | thioxoimidazolidin- | |
| | | 1-yl)-2- | |
| | | (trifluoromethyl) | |
| | | benzonitrile | |
| 15 | 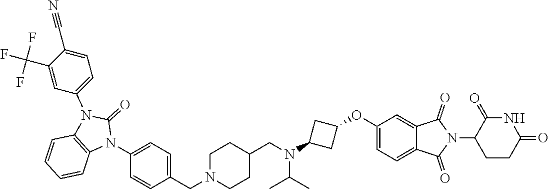 [SMILES: CC(C)N(CC1CCN(Cc2ccc(-n3c(=O)n(-c4ccc(C#N)c(C(F)(F)F)c4)c4ccccc43)cc2)CC1)[C@H]1C[C@H](Oc2ccc3c(c2)C(=O)N(C2CCC(=O)NC2=O)C3=O)C1] | 4-(3-(4-((4- ((((1r,3r)-3-((2- (2,6- dioxopiperidin-3- yl)-1,3- dioxoisoindolin-5- yl)oxy)cyclobutyl) (isopropyl)amino) methyl)piperidin- 1- yl)methyl)phenyl)- 2-oxo-2,3- | 8 and 9 |
| | | dihydro-1H- | |
| | | benzo[d]imidazol- | |
| | | 1-yl)-2- | |
| | | (trifluoromethyl) | |
| | | benzonitrile | |
| 16 | 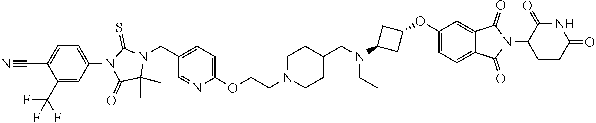 [SMILES: CCN(CC1CCN(CCOc2ccc(CN3C(=S)N(c4ccc(C#N)c(C(F)(F)F)c4)C(=O)C3(C)C)cn2)CC1)[C@H]1C[C@H](Oc2ccc3c(c2)C(=O)N(C2CCC(=O)NC2=O)C3=O)C1] | 4-(3-((6-(2-(4- ((((1r,3r)-3-((2- (2,6- dioxopiperidin-3- yl)-1,3- dioxoisoindolin-5- yl)oxy)cyclobutyl) (ethyl)amino) | 5 |
| | | methyl)piperidin-1- | |
| | | yl)ethoxy)pyridin- | |
| | | 3-yl)methyl)-4,4- | |
| | | dimethyl-5-oxo-2- | |
| | | thioxoimidazolidin- | |
| | | 1-yl)-2- | |
| | | (trifluoromethyl) | |
| | | benzonitrile | |
| 17 | 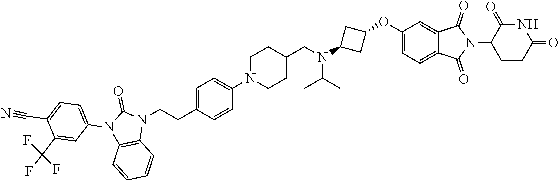 [SMILES: CC(C)N(CC1CCN(c2ccc(CCn3c(=O)n(-c4ccc(C#N)c(C(F)(F)F)c4)c4ccccc43)cc2)CC1)[C@H]1C[C@H](Oc2ccc3c(c2)C(=O)N(C2CCC(=O)NC2=O)C3=O)C1] | 4-(3-(4-(4- ((((1r,3r)-3-((2- (2,6- dioxopiperidin-3- yl)-1,3- dioxoisoindolin-5- yl)oxy)cyclobutyl) (isopropyl)amino) methyl)piperidin- 1-yl)phenethyl)-2- oxo-2,3-dihydro- | 8 and 9 |
| | | 1H- | |
| | | benzo[d]imidazol- | |
| | | 1-yl)-2- | |
| | | (trifluoromethyl) | |
| | | benzonitrile | |
| 18 | 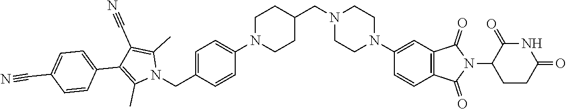 [SMILES: Cc1c(C#N)c(-c2ccc(C#N)cc2)c(C)n1Cc1ccc(N2CCC(CN3CCN(c4ccc5c(c4)C(=O)N(C4CCC(=O)NC4=O)C5=O)CC3)CC2)cc1] | 4-(4- cyanophenyl)-1- (4-(4-((4-(2-(2,6- dioxopiperidin-3- yl)-1,3- dioxoisoindolin-5- yl)piperazin-1- | 11 and 10 |
| | | yl)methyl)piperidin- | |
| | | 1-yl)benzyl)- | |
| | | 2,5-dimethyl-1H- | |
| | | pyrrole-3- | |
| | | carbonitrile | |
| 19 | 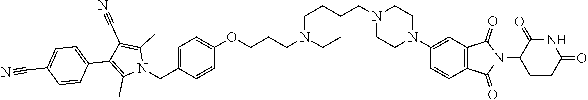 [SMILES: CCN(CCCCN1CCN(c2ccc3c(c2)C(=O)N(C2CCC(=O)NC2=O)C3=O)CC1)CCCOc1ccc(Cn2c(C)c(C#N)c(-c3ccc(C#N)cc3)c2C)cc1] | 4-(4- cyanophenyl)-1- (4-(3-(4-((4-(2- (2,6- dioxopiperidin-3- yl)-1,3- dioxoisoindolin-5- | 11 |
| | | yl)piperazin-1- | |
| | | yl)methyl)piperidin- | |
| | | 1- | |
| | | yl)propoxy)benzyl)- | |
| | | 2,5-dimethyl- | |
| | | 1H-pyrrole-3- | |
| | | carbonitrile | |
| 20 | 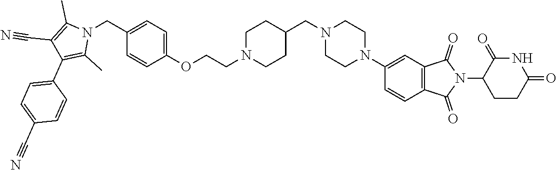 [SMILES: Cc1c(C#N)c(-c2ccc(C#N)cc2)c(C)n1Cc1ccc(OCCN2CCC(CN3CCN(c4ccc5c(c4)C(=O)N(C4CCC(=O)NC4=O)C5=O)CC3)CC2)cc1] | 4-(4- cyanophenyl)-1- (4-(2-(4-((4-(2- (2,6- dioxopiperidin-3- yl)-1,3- dioxoisoindolin-5- yl)piperazin-1- yl)methyl) piperidin-1- yl)ethoxy)benzyl)- | 11 |
| | | 2,5-dimethyl-1H- | |
| | | pyrrole-3- | |
| | | carbonitrile | |
| 21 | 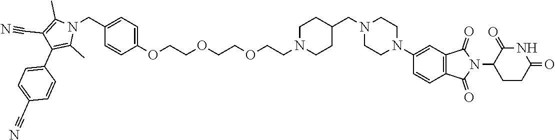 [SMILES: Cc1c(C#N)c(-c2ccc(C#N)cc2)c(C)n1Cc1ccc(OCCOCCOCCN2CCC(CN3CCN(c4ccc5c(c4)C(=O)N(C4CCC(=O)NC4=O)C5=O)CC3)CC2)cc1] | 4-(4- cyanophenyl)-1- (4-(2-(2-(2-(4-((4- (2-(2,6- dioxopiperidin-3- yl)-1,3- dioxoisoindolin-5- yl)piperazin-1- yl)methyl)piperidin- | 11 |
| | | 1- | |
| | | yl)ethoxy)ethoxy) | |
| | | ethoxy)benzyl)- | |
| | | 2,5-dimethyl-1H- | |
| | | pyrrole-3- | |
| | | carbonitrile | |
| 22 | 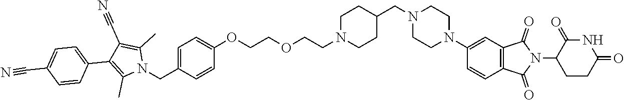 [SMILES: Cc1c(C#N)c(-c2ccc(C#N)cc2)c(C)n1Cc1ccc(OCCOCCN2CCC(CN3CCN(c4ccc5c(c4)C(=O)N(C4CCC(=O)NC4=O)C5=O)CC3)CC2)cc1] | 4-(4- cyanophenyl)-1- (4-(2-(2-(4-((4-(2- (2,6- dioxopiperidin-3- yl)-1,3- dioxoisoindolin-5- | 11 |
| | | yl)piperazin-1- | |
| | | yl)methyl) | |
| | | piperidin-1- | |
| | | yl)ethoxy)ethoxy) | |
| | | benzyl)-2,5- | |
| | | dimethyl-1H- | |
| | | pyrrole-3- | |
| | | carbonitrile | |
| 23 | 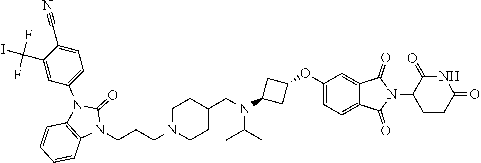 [SMILES: CC(C)N(CC1CCN(CCCn2c(=O)n(-c3ccc(C#N)c(C(F)(F)I)c3)c3ccccc32)CC1)[C@H]1C[C@H](Oc2ccc3c(c2)C(=O)N(C2CCC(=O)NC2=O)C3=O)C1] | 4-(3-(3-(4- ((((1r,3r)-3-((2- (2,6- dioxopiperidin-3- yl)-1,3- dioxoisoindolin-5- yl)oxy)cyclobutyl) (isopropyl)amino) methyl)piperidin- 1-yl)propyl)-2- | 8 and 13 |
| | | oxo-2,3-dihydro- | |
| | | 1H- | |
| | | benzo[d]imidazol- | |
| | | 1-yl)-2- | |
| | | (trifluoromethyl) | |
| | | benzonitrile | |
| 24 | 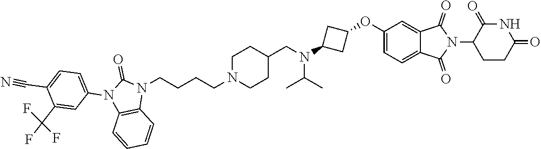 [SMILES: CC(C)N(CC1CCN(CCCCn2c(=O)n(-c3ccc(C#N)c(C(F)(F)F)c3)c3ccccc32)CC1)[C@H]1C[C@H](Oc2ccc3c(c2)C(=O)N(C2CCC(=O)NC2=O)C3=O)C1] | 4-(3-(4-(4- ((((1r,3r)-3-((2- (2,6- dioxopiperidin-3- yl)-1,3- dioxoisoindolin-5- yl)oxy)cyclobutyl) (isopropyl)amino) methyl)piperidin- | 8 and 13 |
| | | 1-yl)butyl)-2-oxo- | |
| | | 2,3-dihydro-1H- | |
| | | benzo[d]imidazol- | |
| | | 1-yl)-2- | |
| | | (trifluoromethyl) | |
| | | benzonitrile | |
| 25 | 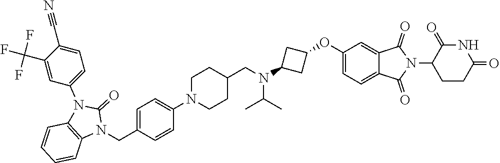 [SMILES: CC(C)N(CC1CCN(c2ccc(Cn3c(=O)n(-c4ccc(C#N)c(C(F)(F)F)c4)c4ccccc43)cc2)CC1)[C@H]1C[C@H](Oc2ccc3c(c2)C(=O)N(C2CCC(=O)NC2=O)C3=O)C1] | 4-(3-(4-(4- ((((1r,3r)-3-((2- (2,6- dioxopiperidin-3- yl)-1,3- dioxoisoindolin-5- yl)oxy)cyclobutyl) (isopropyl)amino) methyl)piperidin- 1-yl)benzyl)-2- | 12 |
| | | oxo-2,3-dihydro- | |
| | | 1H- | |
| | | benzo[d]imidazol- | |
| | | 1-yl)-2- | |
| | | (trifluoromethyl) | |
| | | benzonitrile | |

TABLE 2
Degradation of AR proteins by exemplary heterobifunctional compounds of the present disclosure.

| Comp | Exact | Observed | DC50 | Dmax | |
|---|---|---|---|---|---|
| No. | Mass | Mass One | Code | Code | NMR |
| 1 | 778.25 | 779.46 | A | A | ¹H NMR (300 MHz, DMSO-d₆) δ 8.81 (d, J = 2.1 Hz, 1H), 8.12 (d, J = 2.1 Hz, 1H), 8.04 (s, |
| | | | | | 1H), 7.70 (d, J = 8.4 Hz, 1H), 7.40 (d, J = 8.4 Hz, 2H), 7.29 (d, J = 2.1 Hz, 1H), 7.21 (d, J = |
| | | | | | 8.4 Hz, 2H), 7.08-7.05 (m, 1H), 4.97-4.91 (m, 1H), 3.46 (m, 4H), 2.93-2.55 (m, 13H), 2.44 |
| | | | | | (m, 2H), 2.24-2.12 (m, 2H), 1.77-1.65 (m, 4H), 1.50-1.43 (m, 3H). |
| 2 | 791.28 | 792.47 | A | A | ¹H NMR (300 MHz, DMSO-d₆) δ 10.95 (s, 1H), 8.87 (s, 1H), 8.51 (s, 1H), 7.42-7.36 (m, |
| | | | | | 1H), 7.27-7.04 (m, 6H), 5.10-5.04 (m, 1H), 4.34-4.15 (m, 2H), 3.82-3.78 (m, 2H), 3.27-3.12 |
| | | | | | (m, 4H), 2.94-2.71 (m, 3H), 2.67-2.53 (m, 6H), 2.48-2.31 (m, 3H), 2.23-2.17 (m, 2H), 2.07- |
| | | | | | 1.66 (m, 5H), 1.62-1.41 (m, 1H), 1.32-1.11 (m, 3H). |
| 3 | 841.30 | 842.63 | A | A | ¹H NMR (400 MHz, Methanol-d₄) δ 8.22 (s, 1H), 8.20-8.11 (m, 2H), 7.98 (t, J = 8.0 Hz, |
| | | | | | 2H), 7.80 (d, J = 8.5 Hz, 1H), 7.51 (d, J = 2.2 Hz, 1H), 7.39 (d, J = 8.2 Hz, 1H), 7.10 (d, J = |
| | | | | | 9.2 Hz, 1H), 5.16-5.09 (m, 1H), 5.09 (s, 3H), 4.34 (d, J = 13.3 Hz, 2H), 3.37 (s, 1H), 3.19 |
| | | | | | (s, 3H), 3.12 (d, J = 12.9 Hz, 1H), 2.81-2.71 (m, 2H), 2.29 (s, 1H), 2.14 (s, 1H), 2.00 (d, |
| | | | | | J = 13.0 Hz, 2H), 1.56 (s, 6H), 1.46 (d, J = 11.8 Hz, 1H), 1.31 (s, 1H), 0.12 (s, 1H). |
| 4 | 870.31 | 871.65 | A | C | ¹H NMR (400 MHz, Methanol-d₄) δ 8.24 (d, J = 2.4 Hz, 1H), 8.16 (d, J = 10.3 Hz, 2H), |
| | | | | | 7.97 (d, J = 7.4 Hz, 1H), 7.85-7.74 (m, 2H), 7.31-7.20 (m, 2H), 6.83 (d, J = 8.8 Hz, 1H), |
| | | | | | 5.12 (dd, J = 12.6, 5.5 Hz, 1H), 5.07 (s, 2H), 4.29 (d, J = 13.2 Hz, 2H), 3.37 (s, 2H), 2.88 (t, |
| | | | | | J = 13.0 Hz, 3H), 2.77 (d, J = 12.1 Hz, 1H), 2.74-2.64 (m, 2H), 2.48 (s, 2H), 2.32 (d, J = |
| | | | | | 6.8 Hz, 4H), 2.20 (d, J = 6.9 Hz, 1H), 2.15 (s, 1H), 2.05 (s, 1H), 1.92 (d, J = 12.6 Hz, 2H), |
| | | | | | 1.78 (s, 1H), 1.53 (s, 6H), 1.35 (s, 4H), 1.31 (s, 3H), 1.21 (d, J = 12.0 Hz, 3H), 1.04 (t, J = |
| | | | | | 7.1 Hz, 3H), 0.90 (s, 2H), 0.12 (s, 3H). |
| 5 | 822.25 | 823.46 | A | A | ¹H NMR (400 MHz, DMSO-d₆) δ 11.08 (s, 1H), 8.28 (s, 1H), 8.16 (d, J = 8.4 Hz, 1H), 7.95 |
| | | | | | (d, J = 1.6 Hz, 1H), 7.70-7.65 (m, 2H), 7.34 (s, 1H), 7.27 (br d, J = 8.4 Hz, 1H), 7.17-7.10 |
| | | | | | (m, 1H), 7.06-7.01 (m, 1H), 6.86-6.78 (m, 1H), 5.07 (dd, J = 5.6, 13.2 Hz, 1H), 3.67- |
| | | | | | 3.42 (m, 5H), 3.24-3.00 (m, 2H), 2.95-2.82 (m, 1H), 2.60 (br s, 6H), 2.42 (br s, 4H), 2.28 |
| | | | | | (br s, 2H), 2.17-1.87 (m, 4H), 1.76-1.45 (m, 5H). |
| 6 | 818.30 | 819.51 | B | A | ¹H NMR (400 MHz, DMSO-d₆) δ 11.08 (s, 1H), 8.20 (s, 1H), 7.89 (d, J = 8.8 Hz, 1H), 7.68 |
| | | | | | (d, J = 8.8 Hz, 1H), 7.39 (s, 1H), 7.35 (s, 1H), 7.27 (br d, J = 8.8 Hz, 1H), 7.21-7.13 (m, |
| | | | | | 2H), 7.05 (br d, J = 8.8 Hz, 1H), 6.82 (t, J = 9.6 Hz, 1H), 5.07 (dd, J = 4.4, 12.8 Hz, 1H), |
| | | | | | 3.92 (s, 3H), 3.60 (br s, 1H), 3.45 (br s, 7H), 3.13 (br s, 1H), 2.87 (br d, J = 13.6 Hz, 1H), |
| | | | | | 2.63-2.54 (m, 3H), 2.44-2.36 (m, 5H), 2.28 (br s, 2H), 2.19-2.07 (m, 2H), 2.06-1.92 |
| | | | | | (m, 2H), 1.72-1.44 (m, 5H). |
| 7 | 800.31 | 801.51 | B | B | ¹H NMR (400 MHz, DMSO-d₆) δ 11.07 (s, 1H), 8.17 (s, 1H), 7.88 (d, J = 8.4 Hz, 1H), 7.68 |
| | | | | | (d, J = 8.4 Hz, 1H), 7.41 (d, J = 1.6 Hz, 1H), 7.35 (d, J = 2.0 Hz, 1H), 7.26 (dd, J = 2.0, 8.8 Hz, |
| | | | | | 1H), 7.19 (dd, J = 1.6, 8.0 Hz, 1H), 7.15 (d, J = 8.8 Hz, 2H), 6.64 (d, J = 8.8 Hz, 2H), 5.07 (dd, |
| | | | | | J = 5.2, 12.8 Hz, 1H), 3.92 (s, 3H), 3.55-3.47 (m, 2H), 3.47-3.43 (m, 4H), 3.02-2.81 (m, |
| | | | | | 2H), 2.62-2.56 (m, 2H), 2.56-2.53 (m, 4H), 2.53-2.51 (m, 4H), 2.43 (d, J = 8.8 Hz, 4H), |
| | | | | | 2.23-2.13 (m, 1H), 2.06-1.99 (m, 1H), 1.98-1.90 (m, 1H), 1.73-1.60 (m, 3H), 1.59- |
| | | | | | 1.48(m, 1H). |
| 8 | 870.28 | 871.61 | B | C | |
| 9 | 804.26 | 805.47 | B | A | ¹H NMR (400 MHz, DMSO-d₆) δ 11.07 (s, 1H), 8.18 (s, 1H), 8.15 (d, J = 8.4 Hz, 1H), 7.97 |
| | | | | | (d, J = 1.6 Hz, 1H), 7.73-7.65 (m, 2H), 7.34 (s, 1H), 7.30-7.23 (m, 1H), 7.14 (d, J = 8.8 Hz, |
| | | | | | 2H), 6.64 (d, J = 8.8 Hz, 2H), 5.07 (dd, J = 5.6, 12.8 Hz, 1H), 3.53-3.43 (m, 6H), 3.01-2.82 |
| | | | | | (m, 2H), 2.62-2.56 (m, 2H), 2.56-2.53 (m, 4H), 2.53-2.51 (m, 4H), 2.44-2.37 (m, 4H), |
| | | | | | 2.22-2.13 (m, 1H), 2.05-1.98 (m, 1H), 1.98-1.89 (m, 1H), 1.72-1.60 (m, 3H), 1.58- |
| | | | | | 1.48 (m, 1H). |
| 10 | 838.25 | 839.46 | B | A | ¹H NMR (400 MHz, DMSO-d₆) δ 11.08 (s, 1H), 9.07 (br s, 1H), 8.88 (d, J = 2.0 Hz, 1H), |
| | | | | | 8.52 (d, J = 2.0 Hz, 1H), 7.69 (d, J = 8.4 Hz, 1H), 7.55-7.46 (m, 1H), 7.45-7.36 (m, 2H), |
| | | | | | 7.26 (br dd, J = 9.6, 16.5 Hz, 2H), 5.07 (dd, J = 5.6, 12.8 Hz, 1H), 4.94-4.62 (m, 1H), 4.12 |
| | | | | | (br d, J = 12.8 Hz, 2H), 3.63 (br s, 1H), 3.23-2.82 (m, 8H), 2.66-2.54 (m, 4H), 2.46-2.34 |
| | | | | | (m, 3H), 2.15 (br s, 3H), 2.06-1.92 (m, 3H), 1.86 (br d, J = 11.2 Hz, 2H), 1.56 (br d, J = |
| | | | | | 9.2 Hz, 1H), 1.29 (br d, J = 10.4 Hz, 2H). |
| 11 | 790.28 | 791.49 | B | C | ¹H NMR (300 MHz, DMSO-d₆) 10.96 (s, 1H), 8.91 (d, J = 1.8 Hz, 1H), 8.55 (d, J = 2.1 Hz, |
| | | | | | 1H), 7.50 (d, J = 8.1 Hz , 2H), 7.41 (d, J = 8.4 Hz, 1H), 7.33 (d, J = 8.4 Hz , 2H), 7.28-7.24 |
| | | | | | (m, 1H), 7.17 (s, 1H), 5.13-5.06 (m, 1H), 4.36-4.17 (m, 2H), 3.79-3.74 (m, 2H), 3.02-2.87 |
| | | | | | (m, 3H), 2.77-2.69 (m, 2H), 2.61-2.57 (m, 4H), 2.45-2.35 (m, 3H), 2.23-2.20 (m, 2H), |
| | | | | | 2.06-1.95 (m, 4H), 1.86-1.81 (m, 4H), 1.74-1.70 (m, 2H), 1.56-1.52 (m, 1H), 1.26-1.23 (m, |
| | | | | | 3H). |
| 12 | 855.28 | 856.48 | B | B | ¹H NMR (300 MHz, DMSO-d₆) δ 11.01 (s, 1H), 9.19 (s, 1H), 8.73 (s, 1H), 8.19 (s, 1H), |
| | | | | | 7.74-7.58 (m, 2H), 7.29-7.16 (m, 2H), 7.00 (d, J = 9 Hz, 1H), 5.05-5.03 (m, 2H), 4.04-4.00 |
| | | | | | (d, J = 11.7 Hz, 2H), 2.99-2.80 (m, 3H), 2.71-2.54 (m, 5H), 2.41-2.40 (m, 2H), 2.24-2.15 (m, |
| | | | | | 4H), 2.05-2.00 (m, 5H), 1.81-1.68 (m, 3H), 1.60-1.53 (m, 3H), 1.23-1.09 (m, 2H). |
| 13 | 883.31 | 884.53 | B | C | ¹H NMR (300 MHz, DMSO-d₆) δ 11.03 (s, 1H), 9.22 (s, 1H), 8.76 (s, 1H), 8.07 (s, 1H), |
| | | | | | 7.83 (d, J = 8.1 Hz, 1H), 7.50 (d, J = 6.3 Hz, 1H), 7.29-7.26 (m, 2H), 7.00 (d, J = 6.3 Hz, |
| | | | | | 1H), 5.15-5.10 (m, 1H), 4.92-4.90 (m, 1H), 4.44-4.42 (m, 2H), 3.68-3.59 (m, 1H), 2.95-2.91 |
| | | | | | (m, 4H), 2.62-2.50 (m, 7H), 2.28-2.08 (m, 4H), 2.05-1.85 (m, 5H), 1.78-1.63 (m, 2H), |
| | | | | | 1.15-1.13 (m, 2H), 1.06-0.95 (m, 6H). |
| 14 | 885.32 | 886.67 | B | B | ¹H NMR (400 MHz, Methanol-d₄) δ 8.35 (d, J = 2.4 Hz, 1H), 8.16 (d, J = 9.8 Hz, 2H), 7.97 |
| | | | | | (dd, J = 8.5, 2.3 Hz, 2H), 7.76 (d, J = 8.5 Hz, 1H), 7.46 (s, 1H), 7.33 (d, J = 8.8 Hz, 1H), |
| | | | | | 6.92 (d, J = 8.5 Hz, 1H), 5.17-5.06 (m, 3H), 4.73 (s, 2H), 3.79 (s, 3H), 3.63 (s, 5H), 3.37 |
| | | | | | (s, 1H), 2.91-2.82 (m, 0H), 2.81-2.70 (m, 1H), 2.18 (d, J = 15.8 Hz, 4H), 1.63 (s, 2H), |
| | | | | | 1.54 (s, 6H), 1.31 (s, 3H), 0.90 (s, 2H), 0.12 (s, 4H), 0.14-0.07 (m, 1H). |
| 15 | 873.35 | 874.70 | B | C | ¹H NMR (300 MHz, DMSO-d₆) δ 11.11 (s, 1H), 8.43-8.35 (m, 2H), 8.24-8.22 (m, 1H), |
| | | | | | 7.86-7.82 (m, 1H), 7.58-7.54 (m, 4H), 7.27-7.12 (m, 6H), 5.14-5.09 (m, 1H), 4.90 (s, 1H), |
| | | | | | 3.67-3.62 (m, 1H), 3.55 (s, 2H), 2.88-2.86 (m, 4H), 2.55-2.50 (s, 1H), 2.25-2.22 (m, 2H), |
| | | | | | 2.18-1.93(m, 7H), 1.78-1.72 (m, 2H), 1.34 (m, 1H), 1.23-1.20 (m, 3H), 0.92 (m, 6H). |
| 16 | 914.34 | 915.69 | B | C | ¹H NMR (400 MHz, Methanol-d₄) δ 8.29 (d, J = 2.6 Hz, 1H), 8.16 (d, J = 6.9 Hz, 2H), 7.98 |
| | | | | | (d, J = 7.0 Hz, 1H), 7.95-7.88 (m, 1H), 7.82 (d, J = 8.3 Hz, 1H), 7.30-7.20 (m, 2H), 6.83 |
| | | | | | (d, J = 8.6 Hz, 1H), 5.14 (s, 3H), 5.10 (d, J = 5.3 Hz, 1H), 4.48 (t, J = 5.5 Hz, 2H), 3.53 (s, |
| | | | | | 2H), 3.37 (s, 2H), 3.09 (s, 2H), 2.86 (s, 3H), 2.80-2.70 (m, 1H), 2.69-2.60 (m, 1H), 2.46 |
| | | | | | (s, 1H), 2.35-2.26 (m, 3H), 2.21 (s, 1H), 1.86 (d, J = 12.9 Hz, 2H), 1.53 (s, 6H), 1.40 (s, |
| | | | | | 1H), 1.31 (s, 5H), 1.02 (t, J = 7.1 Hz, 3H), 0.90 (s, 5H), 0.12 (s, 6H), 0.09 (d, J = 11.4 Hz, |
| | | | | | 1H). |
| 17 | 887.36 | 888.70 | C | C | ¹H NMR (300 MHz, DMSO-d₆) δ 11.08 (s, 1H), 8.37-8.35 (m, 1H), 8.22 (s, 1H), 8.15-8.12 |
| | | | | | (m, 1H), 7.85-7.82 (d, 1H),7.34-7.06 (m, 8H), 6.84-6.81 (m, 2H), 5.14-5.10 (m, 1H), 4.91 |
| | | | | | (m, 1H), 4.10-4.05 (m, 2H), 3.65-3.61 (m, 3H), 2.92-2.91 (m, 3H), 2.56-2.51 (m, 2H), 2.43 |
| | | | | | (m, 2H), 2.27-2.15 (m, 2H), 2.10-1.94 (m, 1H) 1.83-1.79 (m, 2H), 1.46-1.35 (m, 1H), 1.24- |
| | | | | | 1.08 (m, 6H), 0.94-0.80 (d, 7H). |
| 18 | 748.35 | 749.55 | D | C | ¹H NMR (300 MHz, DMSO-d₆) δ 11.07 (s, 1H), 8.23-7.84 (m, 2H), 7.66 (d, J = 8.5 Hz, |
| | | | | | 1H), 7.60-7.50 (m, 2H), 7.32-7.18 (m, 2H), 6.88 (d, J = 2.3 Hz, 4H), 5.14-4.99 (m, 3H), |
| | | | | | 3.64 (d, J = 12.1 Hz, 2H), 3.59-3.03 (m, 6H),2.90-2.77 (m, 1H), 2.69-2.45 (m, 6H), 2.33 (s, |
| | | | | | 3H), 2.20-2.16 (m, 5H), 2.02-1.98 (m, 1H), 1.78-1.67 (m, 3H), 1.32-1.05 (m, 2H). |
| 19 | 806.39 | 807.61 | D | C | ¹H NMR (300 MHz, CD₃OD) δ 7.79 (d, J = 8.3 Hz, 2H), 7.68 (d, J = 8.5 Hz, 1H), 7.58 (d, |
| | | | | | J = 8.3 Hz, 2H), 7.36 (s, 1H), 7.23 (d, J = 8.3 Hz, 1H), 6.93 (s, 4H), 5.18 (s, 2H), 5.07 (dd, J = |
| | | | | | 12.4, 5.3 Hz, 1H), 4.02 (t, J = 6.0 Hz, 2H), 3.46 (m, 4H), 3.05 (m, 2H), 2.82 (dd, J = 17.6, |
| | | | | | 12.5 Hz, 2H), 2.73 (m, 1H), 2.59 (m, 5H), 2.38 (m, 3H), 2.33-2.19 (m, 5H), 2.12 (m, 3H), |
| | | | | | 2.01 (m, 2H), 1.85 (m, 2H), 1.67 (m, 1H), 1.45 (m, 1H), 1.30 (m, 2H). |
| 20 | 792.37 | 793.59 | D | C | ¹H NMR (400 MHz, CDCl₃) δ 8.09 (brs, 1H), 7.74-7.70 (m, 3H), 7.54 (d, J = 7.9 Hz, 2H), |
| | | | | | 7.10-7.06 (m, 1H), 6.93-6.89 (m, 4H), 5.06 (s, 2H), 4.95-4.92 (m, 1H), 4.17 (brs, 2H), 3.45- |
| | | | | | 3.40 (m, 4H), 3.11 (brs, 2H), 2.95-2.79 (m, 4H), 2.76-2.57 (m, 4H), 2.39 (s, 3H), 2.30-2.15 |
| | | | | | (m, 7H), 1.88-1.80 (m, 3H), 1.54-1.10 (m, 4H). |
| 21 | 880.43 | 881.66 | D | C | ¹H NMR (300 MHz, DMSO-d₆) δ 11.05 (s, 1H), 7.92-7.83 (m, 2H), 7.70-7.40 (m, 3H), |
| | | | | | 7.33-7.16 (m, 2H), 7.07-6.81 (m, 4H), 5.16 (s, 2H), 5.15-4.90(m, 1H), 4.15-4.00 (m, 2H), |
| | | | | | 3.80-3.60 (m, 2H), 3.60-3.34 (m, 10H), 2.95-2.75 (m, 3H), 2.60-2.55 (m, 1H), 2.48-2.35 (m, |
| | | | | | 7H), 2.35-2.30 (m, 3H), 2.20-2.15 (s, 3H), 2.15-2.10 (m, 2H), 2.05-1.75 (m, 3H), 1.67-1.55 |
| | | | | | (m, 2H), 1.55-1.35 (m, 1H), 1.20-0.85 (m, 2H). |
| 22 | 836.40 | 837.62 | D | C | ¹H NMR (300 MHz, DMSO-d₆) δ 11.06 (s, 1H), 7.93-7.84 (m, 2H), 7.65 (d, J = 8.5 Hz, |
| | | | | | 1H), 7.60-7.50 (m, 2H), 7.34-7.17 (m, 2H), 7.00-6.86 (m, 4H), 5.16 (s, 2H), 5.05 (dd, J = |
| | | | | | 12.7, 5.3 Hz, 1H), 4.04 (m, 2H), 3.68 (dd, J = 5.6, 3.6 Hz, 2H), 3.52 (m, 2H), 3.50-3.34 |
| | | | | | (m, 4H), 2.87-2.77 (m, 3H), 2.66-2.56 (m, 2H), 2.42 (m, 5H), 2.32 (s, 3H), 2.26-2.12 |
| | | | | | (m, 5H), 2.11-1.86 (m, 4H), 1.62 (d, J = 12.7 Hz, 2H), 1.45 (s, 1H), 1.15-0.99 (m, 2H). |
| 23 | 825.35 | 826.55 | D | C | ¹H NMR (400 MHz, DMSO-d₆) δ 11.08 (s, 1H), 8.37-8.35 (m, 1H), 8.28-8.27 (m, 1H), |
| | | | | | 8.18-8.15 (m, 1H), 7.84-7.82 (m, 1H), 7.39-7.32 (m, 2H), 7.27-7.21 (m, 3H), 7.15-7.13 (m, |
| | | | | | 1H), 5.13-5.09 (m, 1H), 4.88 (m, 1H), 3.98-3.95 (m, 2H), 3.61 (m, 1H), 2.87-2.75 (m, 4H), |
| | | | | | 2.57-2.50 (m, 2H), 2.34-2.31 (m, 4H), 2.15-2.07 (m, 6H), 1.88-1.62 (m, 6H), 1.27-1.15 (m, |
| | | | | | 2H), 0.91-0.87 (m, 6H). |
| 24 | 839.36 | 840.56 | D | C | ¹H NMR (400 MHz, DMSO-d₆) δ 11.19-11.01 (s, 1H), 8.38-8.17 (m, 3H), 7.83-7.73 (s, |
| | | | | | 1H), 7.73-6.85 (m, 6H), 5.12-4.68 (m, 2H), 4.10-3.77 (m, 2H), 3.62-3.50 (m, 1H), 2.81- |
| | | | | | 2.70 (m, 4H), 2.39-2.07 (m, 9H), 1.91-1.23 (m, 13H), 0.99-0.60 (m, 6H). |
| 25 | 873.35 | 874.68 | D | C | ¹H NMR (400 MHz, Methanol-d₄) δ 8.27-8.13 (m, 3H), 7.85 (s, 1H), 7.61-7.04 (m, 10H), |
| | | | | | 3.91-3.54 (m, 4H), 2.91-2.50 (m, 11H), 2.36-1.93 (m, 6H), 1.62-0.71 (m, 19H). |

A novel bifunctional molecule, which contains a recruiting moiety that selectively or preferentially binds to a AR protein and an E3 ubiquitin ligase recruiting moiety is described. The bifunctional molecules of the present disclosure actively ubiquitinate the mutated AR, resulting in proteasomal degradation, leading to suppression of cellular proliferation and induction of apoptosis.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A hetero-bifunctional compound having the chemical structure:
PTM-L-CLM,
or a pharmaceutically acceptable salt or solvate thereof,
wherein:
(a) the CLM is a small molecule E3 ubiquitin ligase binding moiety that binds a cereblon E3 ubiquitin ligase, and is represented by the chemical structure: 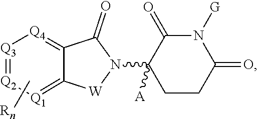
wherein:
W is selected from the group consisting of CH₂, C═O, NH, and N-alkyl;
G is H or an unsubstituted linear or branched C₁₋₃ alkyl;
Q₁, Q₂, Q₃, and Q₄ represent a N or a C substituted with a group selected from H and R;
A is independently selected from the group H, unsubstituted linear or branched C₁₋₃ alkyl, C₁, and F;
n is an integer from 1 to 4;
R is selected from the group consisting of H, NH₂, an unsubstituted or substituted linear or branched C₁₋₄ alkyl, —OR′, —Cl, —F, —Br, —CF₃, and —CN, wherein an R is the point of attachment or an R is modified to be covalently joined to the chemical linking group (L);
R′ is independently selected from the group consisting of H and an unsubstituted or substituted C₁₋₃ alkyl; and [symbol]
represents a bond that may be stereospecific ((R) or (S)) or non-stereospecific;
(b) the PTM is a small molecule androgen receptor (AR) targeting moiety that binds to the androgen receptor, and is represented by the chemical structure: 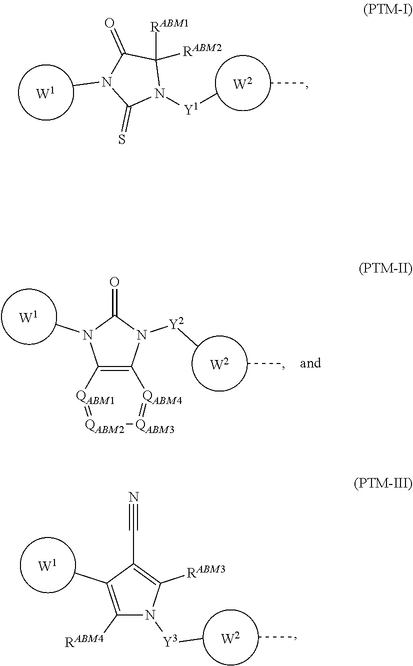
wherein:
W¹ is a 5- or 6-membered aromatic group with 0 to 2 heteroatoms substituted with a CN group and optionally substituted with one or more of H, halogen, hydroxyl, an unsubstituted or substituted linear or branched C₁₋₄ alkyl, an unsubstituted or substituted linear or branched C₁₋₄ alkoxyl, C₁₋₄ haloalkyl;
Y¹ is a bond or a C1-C2 alkyl, optionally substituted with a methyl, OH, or a halogen;
Y² is a bond or a C1-C5 alkyl, optionally substituted with a methyl, OH, or a halogen;
Y³ is a C1-C2 alkyl, optionally substituted with a methyl, OH, or a halogen;
R^{ABM1} and R^{ABM2} are each independently an unsubstituted or substituted C1-C3 alkyl; or R^{ABM1} and R^{ABM2} together with the carbon to which they are attached form an unsubstituted or substituted C3-C5 membered ring;
R^{ABM3} and R^{ABM4} are each independently a H or an unsubstituted or substituted C1-C3 alkyl;
W² is a bond or a 5-7 membered aromatic group with 0 to 2 heteroatoms, optionally substituted by 1 or 2 R^{W2};
each of Q_{PTM1}, Q_{PTM2}, Q_{PTM3}, and Q_{PTM4} represent a N or a C substituted with a group selected from H and R;
each R^{W2} is independently: H; OH; NH₂; halogen; linear or branched C₁₋₃ alkyl optionally substituted by 1 or more F; linear or branched C₁₋₃ heteroalkyl optionally substituted by 1 or more F; and OC₁₋₃alkyl optionally substituted by 1 or more —F; and [symbol]
is the linker attachment point; and
(c) the L is a chemical linking group that covalently couples the CLM to the PTM.

2. The compound according to claim 1, wherein the compound is represented by the chemical structure: 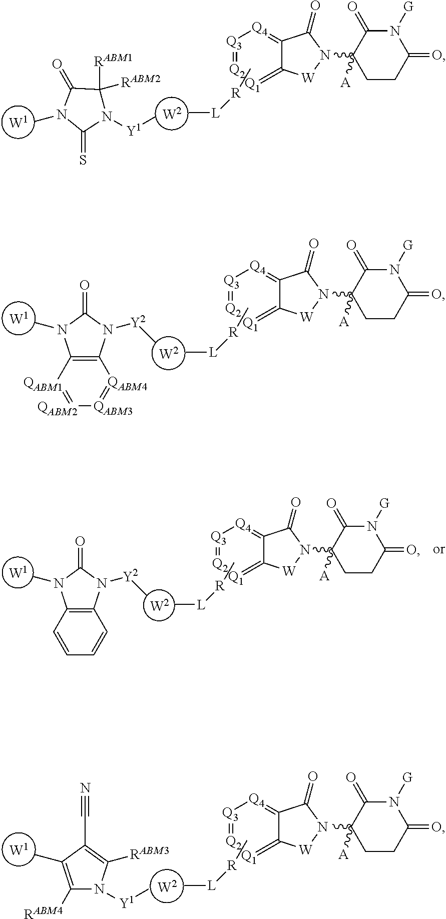
wherein the R that is covalently linked to L is O, N*, or NH, wherein N* is a nitrogen atom that is shared with the chemical linking group.

3. The compound according to claim 1, wherein at least one of:
(i) W¹ is selected from: 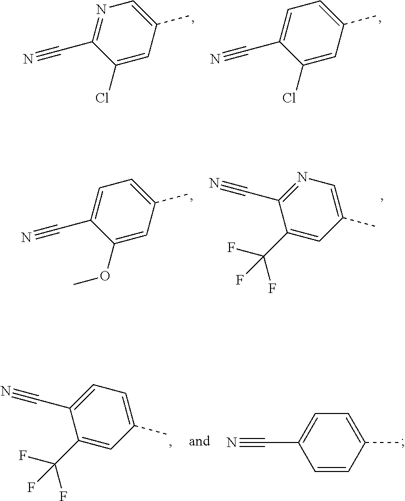
(ii) W² is a bond or selected from: 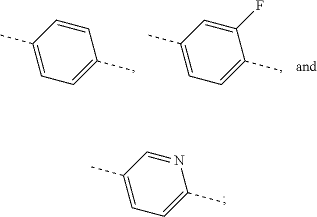
(iii) R^{ABM1} and R^{ABM2} are each a methyl; or R^{ABM1} and R^{ABM2} together with the carbon they are attached form a cyclobutyl group;
(iv) R^{ABM3} and R^{ABM4} are each a methyl; and
(v) combinations thereof,
wherein the dashed lines indicate attachment points.

4. The compound of claim 1, wherein the CLM has a chemical structure represented by: 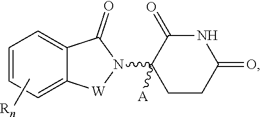
wherein:
W is independently selected from the group CH₂ and C═O;
A is selected from a H or methyl;
n is 1 or 2;
each R is independently selected from a H, OH, Cl, —F, —Br, or methyl, wherein an R is the point of attachment or an R is modified to be covalently joined to the chemical linking group (L); and [symbol]
represents a bond that may be stereospecific ((R) or (S)) or non-stereospecific.

5. The compound of claim 1, wherein the chemical linking group (L) comprises the chemical structure: 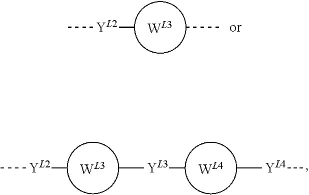
wherein:
Y^{L2} is a bond, O, or a unsubstituted or substituted linear or branched C1-C10 alkyl, wherein one or more C atoms are optionally replaced with O and each carbon is optionally substituted with a halogen, methyl, or ethyl;
W^{L3} is a 3-7 membered ring with 0-3 heteroatoms, optionally substituted with a halogen or methyl;
Y^{L3} is absent or a C1-C4 alkyl, wherein one or more C atoms are optionally replaced with O or NH, and wherein: each carbon is optionally substituted with a halogen or a linear or branched C1-C4 alkyl; and each NH is optionally substituted with a linear or branched C1-C5 alkyl;
W^{L4} is absent or a 3-7 membered ring, each with 0-3 heteroatoms and optionally substituted with halogen, or methyl;
Y_{L4} is bond, O, or an unsubstituted or substituted linear or branched C1-C3 alkyl, wherein a carbon is optionally replaced with O or NH, and optionally substituted with a halogen or methyl; and
each [symbol] of the chemical linking group indicate an attachment point.

6. The compound according to claim 1, wherein the chemical linking group (L) is a means for covalently coupling the PTM to the CLM.

7. The compound according to claim 1, wherein the chemical linking group (L) is selected from the group consisting of: 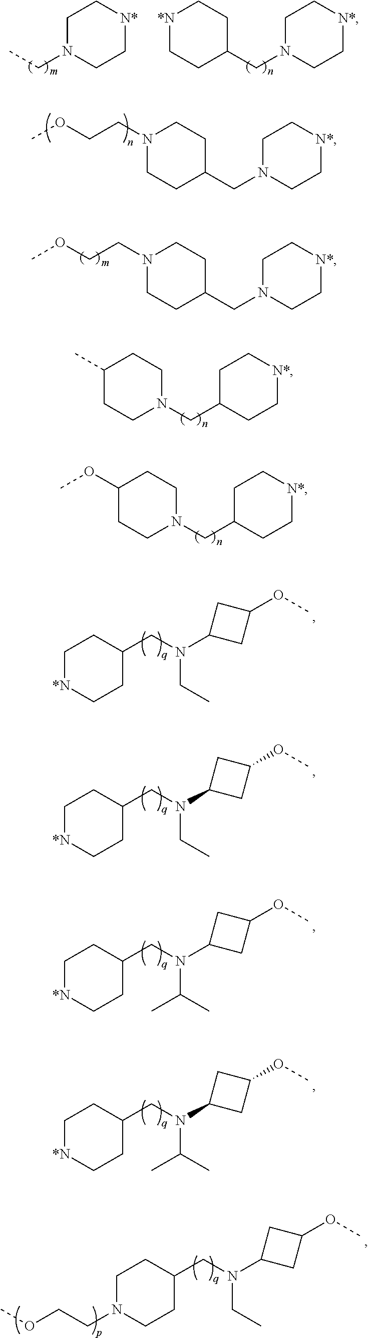 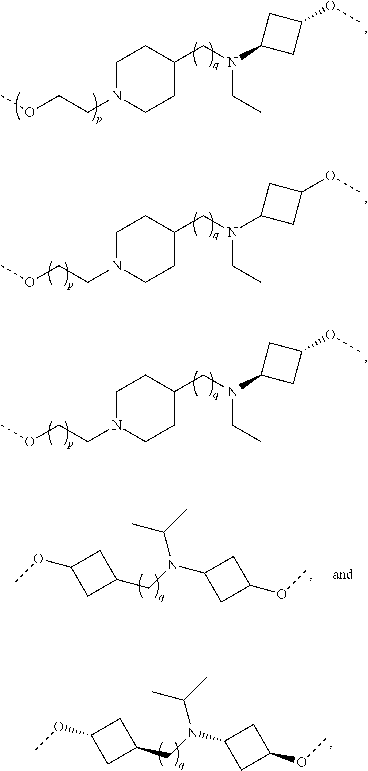
wherein:
m of the chemical linking group is 1, 2, 3, 4, or 5;
n of the chemical linking group is 1, 2, or 3;
p of the chemical linking group is 0 or 1;
q i ofthe chemical linking group s 1 or 2; [symbol]
of the chemical linking group indicates the site that is covalently linked to the CLM or PTM; and
* indicates the site that is covalently linked to the CLM or PTM, or is a nitrogen atom that is shared with the CLM or PTM.

8. The compound according to claim 1, wherein at least one of:
(a) the ULM is represented by: 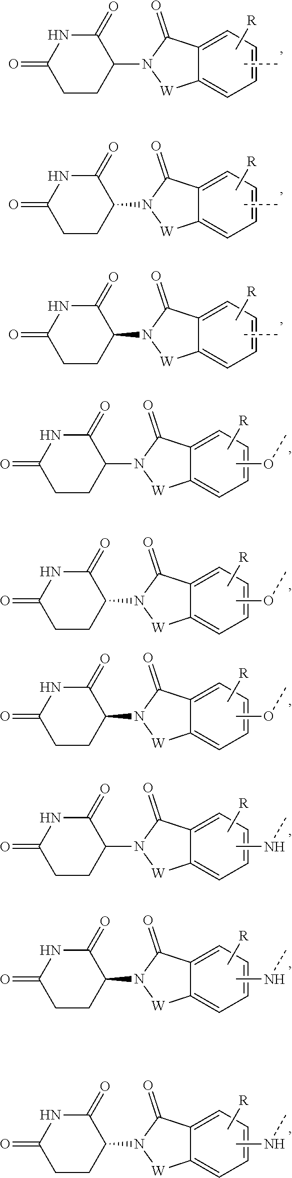 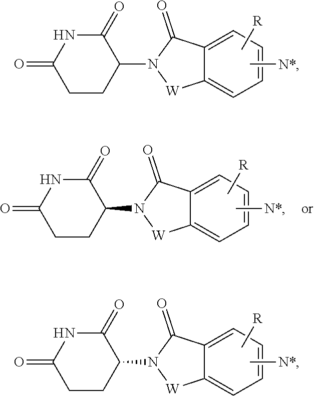
wherein: [symbol]
of the CLM indicates the point of attachment with the chemical linking group; and
N* is a nitrogen atom that is shared with the chemical linking group;
(b) the PTM is represented by: 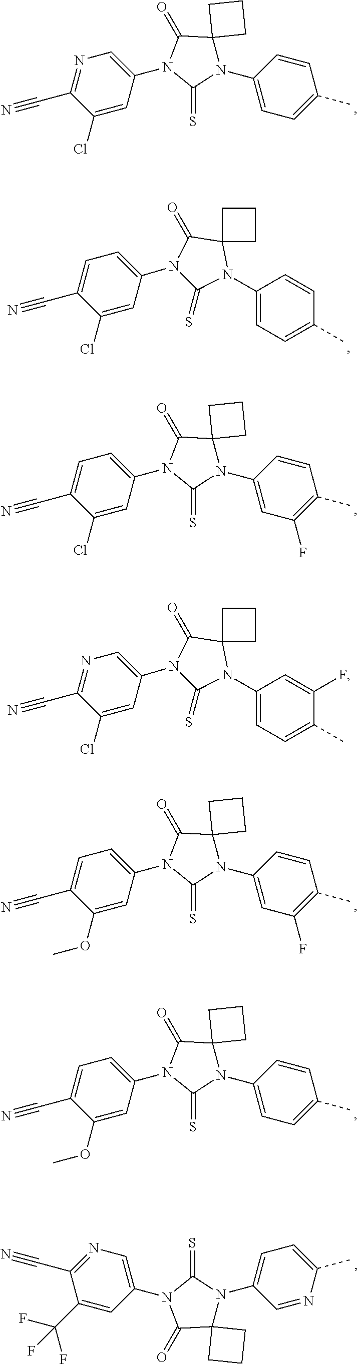 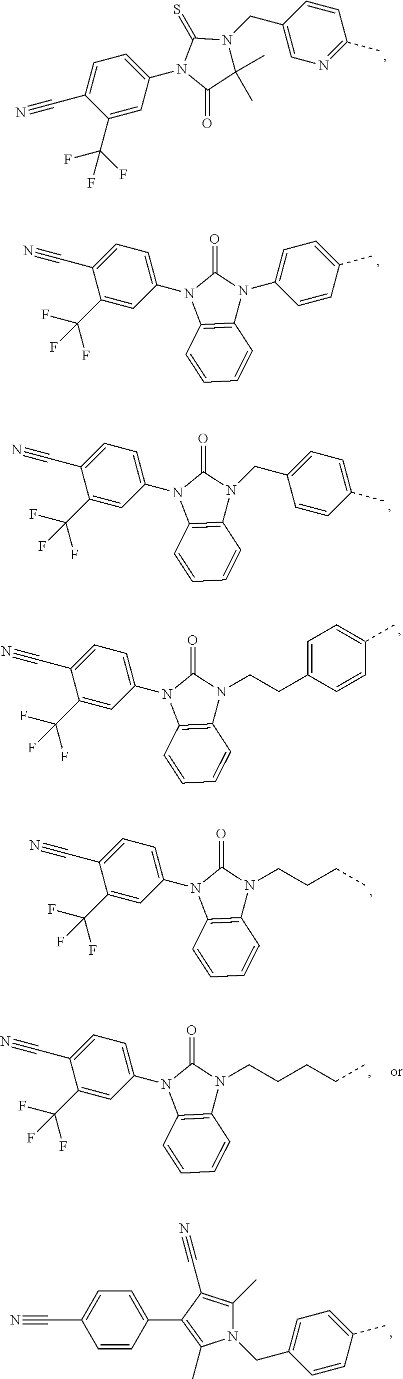
wherein the [symbol] of the PTM indicates the point of attachment with the chemical linking group (L);
(c) the chemical linking group (L) selected from: 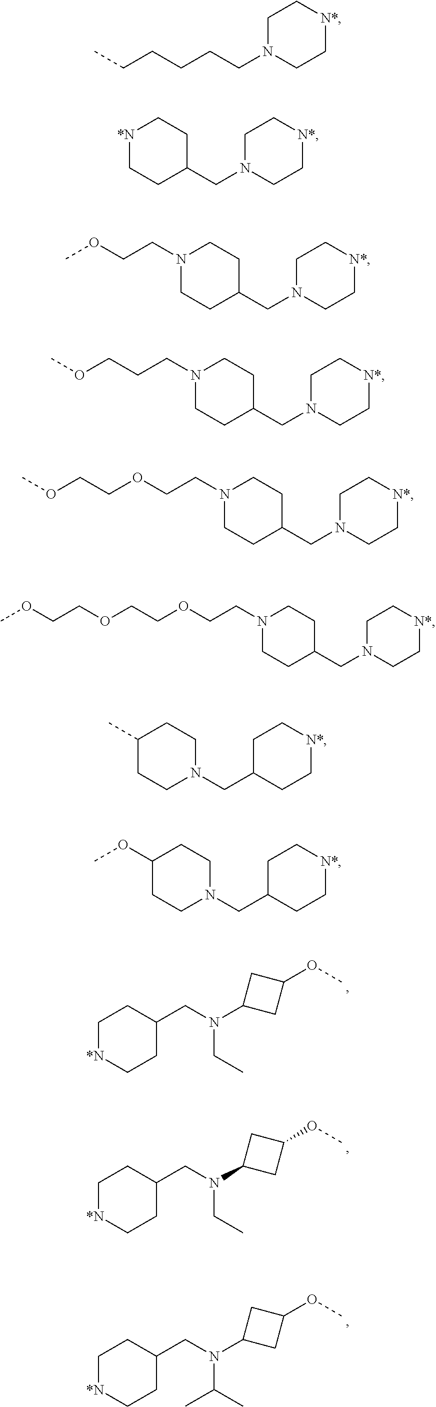 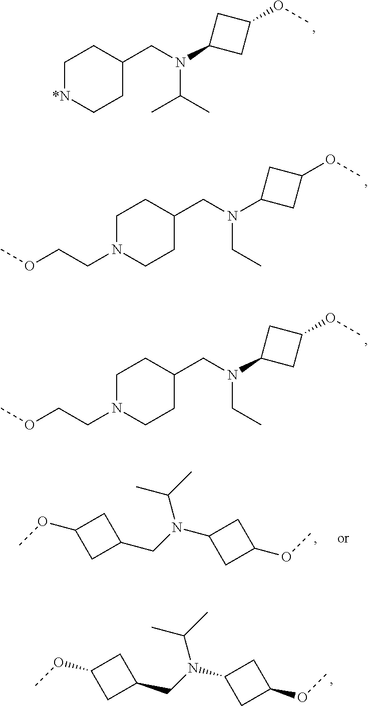
wherein: [symbol]
of the chemical linking group indicates the site that is covalently linked to the ULM or PTM; and
* indicates the site that is covalently linked to the ULM or PTM or is a nitrogen atom that is shared with the ULM or PTM; or
(d) a combination thereof.

9. The compound according to claim 1, wherein at least one of:
the PTM is a PTM selected from a compound of Table 1;
the CLM is a CLM is selected from a compound of Table 1; and
the L is an L selected from a compound of Table 1.

10. The compound of claim 1, wherein the compound is selected from the group consisting of compounds 1-25 of Table 1:

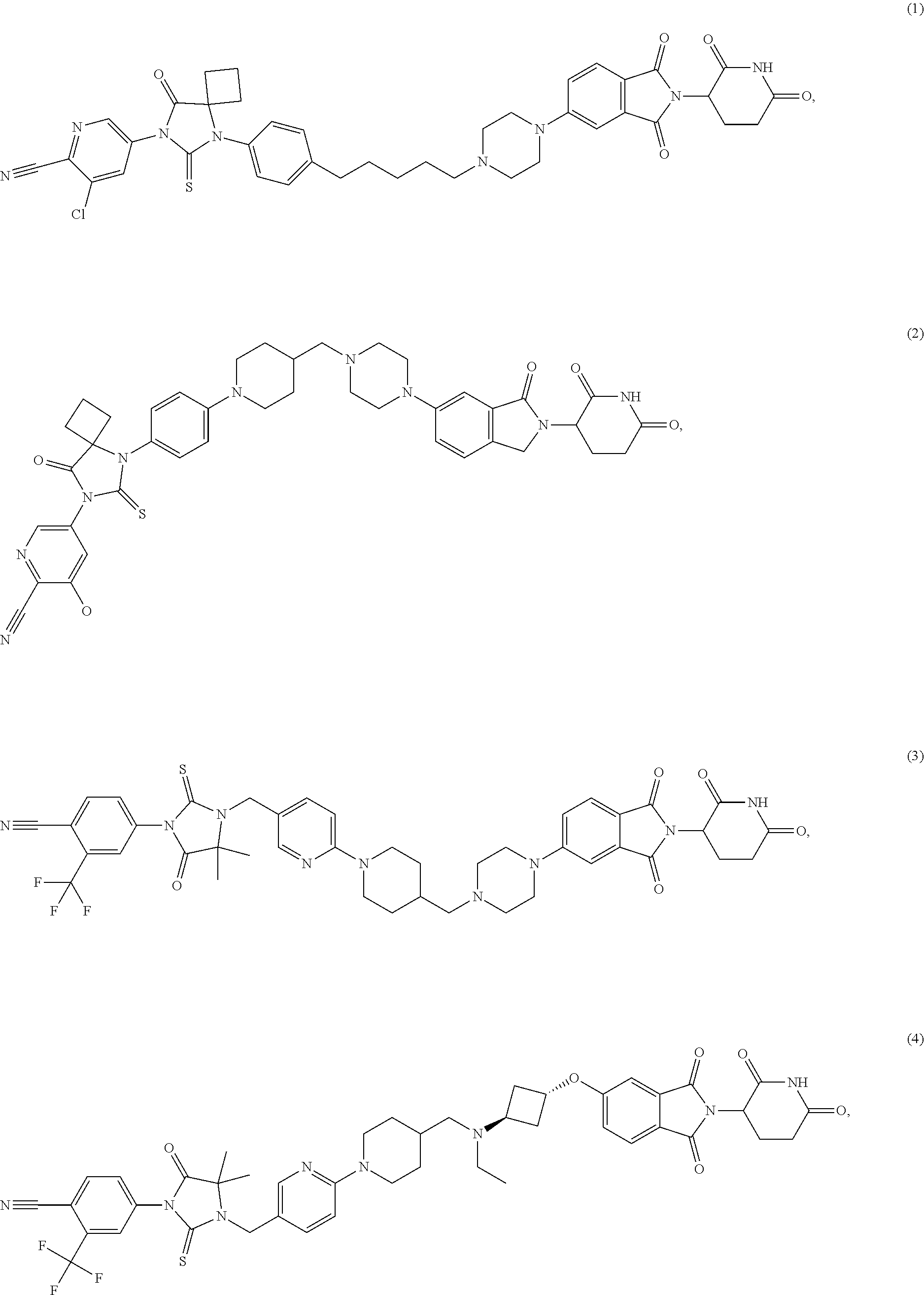 [SMILES: N#Cc1ncc(N2C(=O)C3(CCC3)N(c3ccc(CCCCCN4CCN(c5ccc6c(c5)C(=O)N(C5CCC(=O)NC5=O)C6=O)CC4)cc3)C2=S)cc1Cl ; N#Cc1ncc(N2C(=O)C3(CCC3)N(c3ccc(N4CCC(CN5CCN(c6ccc7c(c6)C(=O)N(C6CCC(=O)NC6=O)C7)CC5)CC4)cc3)C2=S)cc1O ; CC1(C)C(=O)N(c2ccc(C#N)c(C(F)(F)F)c2)C(=S)N1Cc1ccc(N2CCC(CN3CCN(c4ccc5c(c4)C(=O)N(C4CCC(=O)NC4=O)C5=O)CC3)CC2)nc1 ; CCN(CC1CCN(c2ccc(CN3C(=S)N(c4ccc(C#N)c(C(F)(F)F)c4)C(=O)C3(C)C)cn2)CC1)[C@H]1C[C@H](Oc2ccc3c(c2)C(=O)N(C2CCC(=O)NC2=O)C3=O)C1]

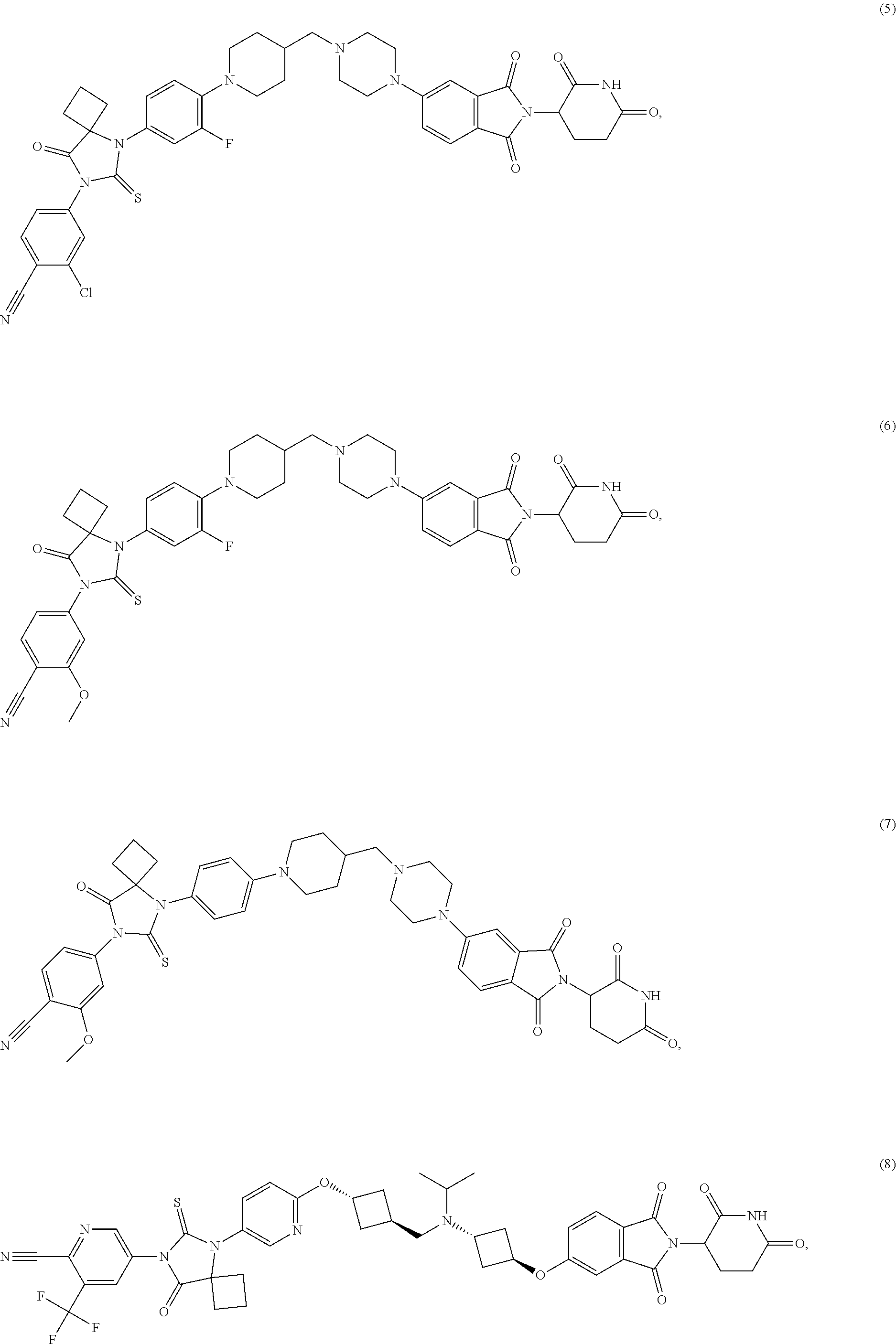 [SMILES: N#Cc1ccc(N2C(=O)C3(CCC3)N(c3ccc(N4CCC(CN5CCN(c6ccc7c(c6)C(=O)N(C6CCC(=O)NC6=O)C7=O)CC5)CC4)c(F)c3)C2=S)cc1Cl ; COc1cc(N2C(=O)C3(CCC3)N(c3ccc(N4CCC(CN5CCN(c6ccc7c(c6)C(=O)N(C6CCC(=O)NC6=O)C7=O)CC5)CC4)c(F)c3)C2=S)ccc1C#N ; COc1cc(N2C(=O)C3(CCC3)N(c3ccc(N4CCC(CN5CCN(c6ccc7c(c6)C(=O)N(C6CCC(=O)NC6=O)C7=O)CC5)CC4)cc3)C2=S)ccc1C#N ; CC(C)N(C[C@H]1C[C@H](Oc2ccc(N3C(=S)N(c4cnc(C#N)c(C(F)(F)F)c4)C(=O)C34CCC4)cn2)C1)[C@H]1C[C@H](Oc2ccc3c(c2)C(=O)N(C2CCC(=O)NC2=O)C3=O)C1]

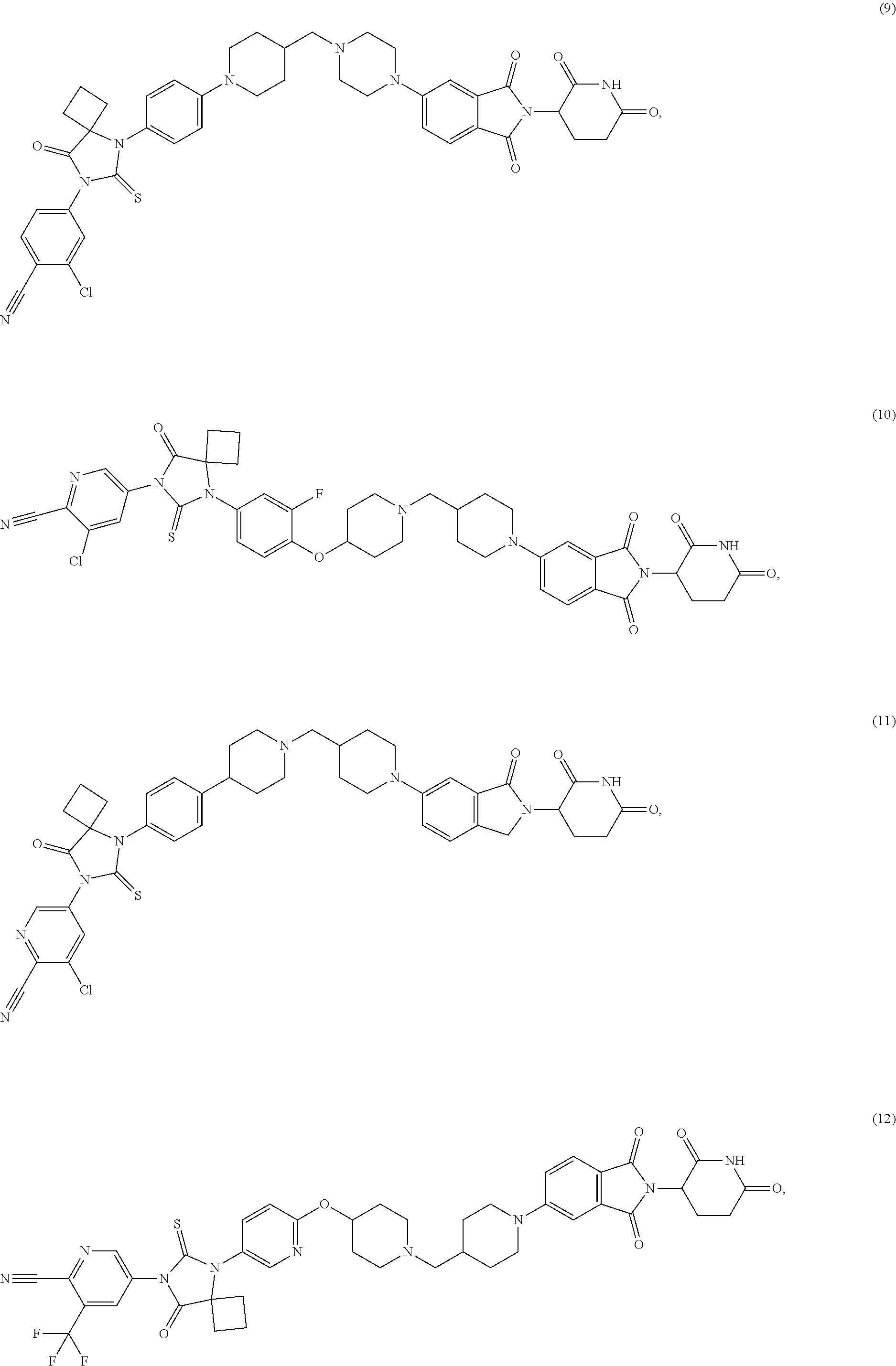 [SMILES: N#Cc1ccc(N2C(=O)C3(CCC3)N(c3ccc(N4CCC(CN5CCN(c6ccc7c(c6)C(=O)N(C6CCC(=O)NC6=O)C7=O)CC5)CC4)cc3)C2=S)cc1Cl ; N#Cc1ncc(N2C(=O)C3(CCC3)N(c3ccc(OC4CCN(CC5CCN(c6ccc7c(c6)C(=O)N(C6CCC(=O)NC6=O)C7=O)CC5)CC4)c(F)c3)C2=S)cc1Cl ; N#Cc1ncc(N2C(=O)C3(CCC3)N(c3ccc(C4CCN(CC5CCN(c6ccc7c(c6)C(=O)N(C6CCC(=O)NC6=O)C7)CC5)CC4)cc3)C2=S)cc1Cl ; N#Cc1ncc(N2C(=O)C3(CCC3)N(c3ccc(OC4CCN(CC5CCN(c6ccc7c(c6)C(=O)N(C6CCC(=O)NC6=O)C7=O)CC5)CC4)nc3)C2=S)cc1C(F)(F)F]

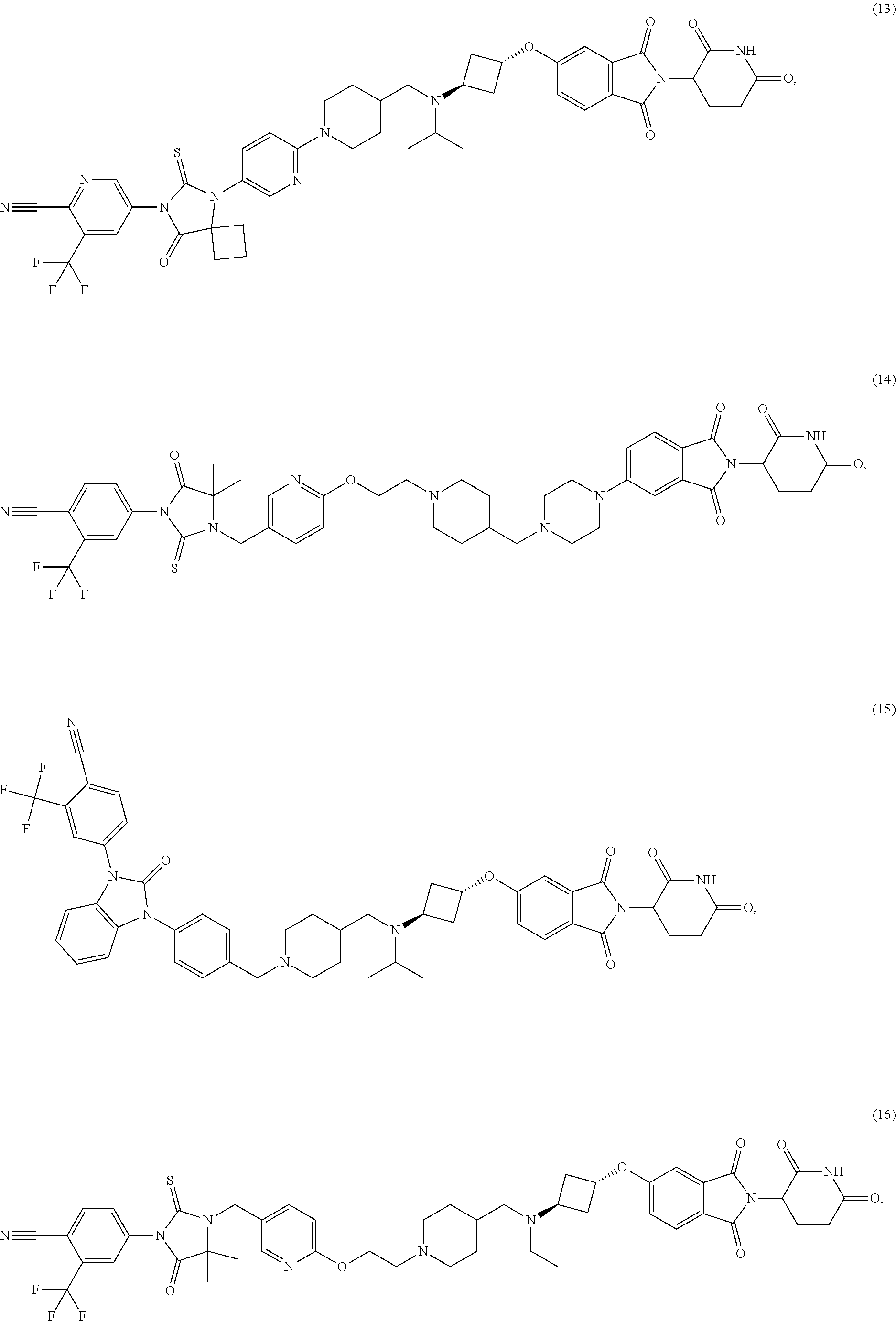

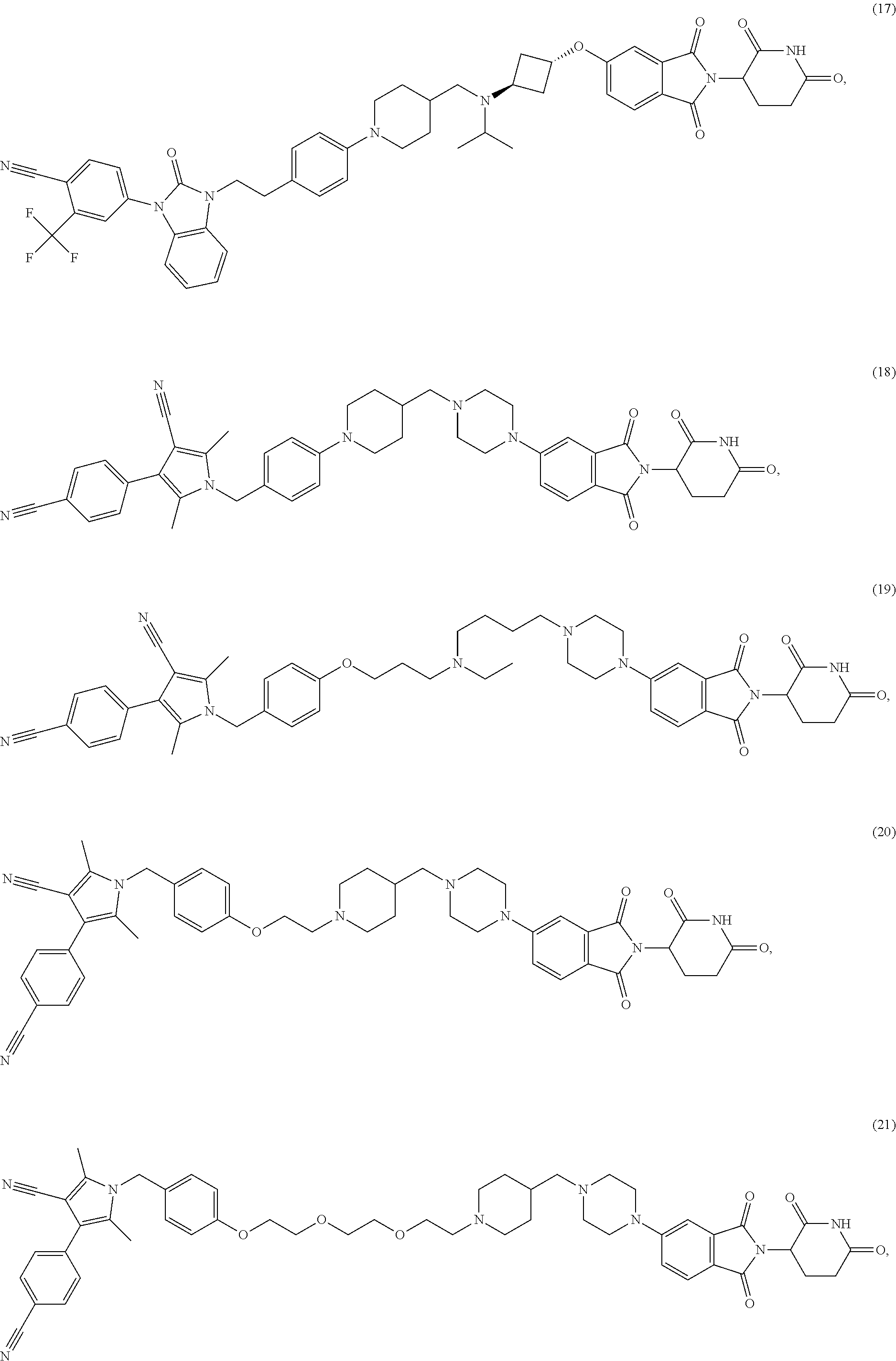

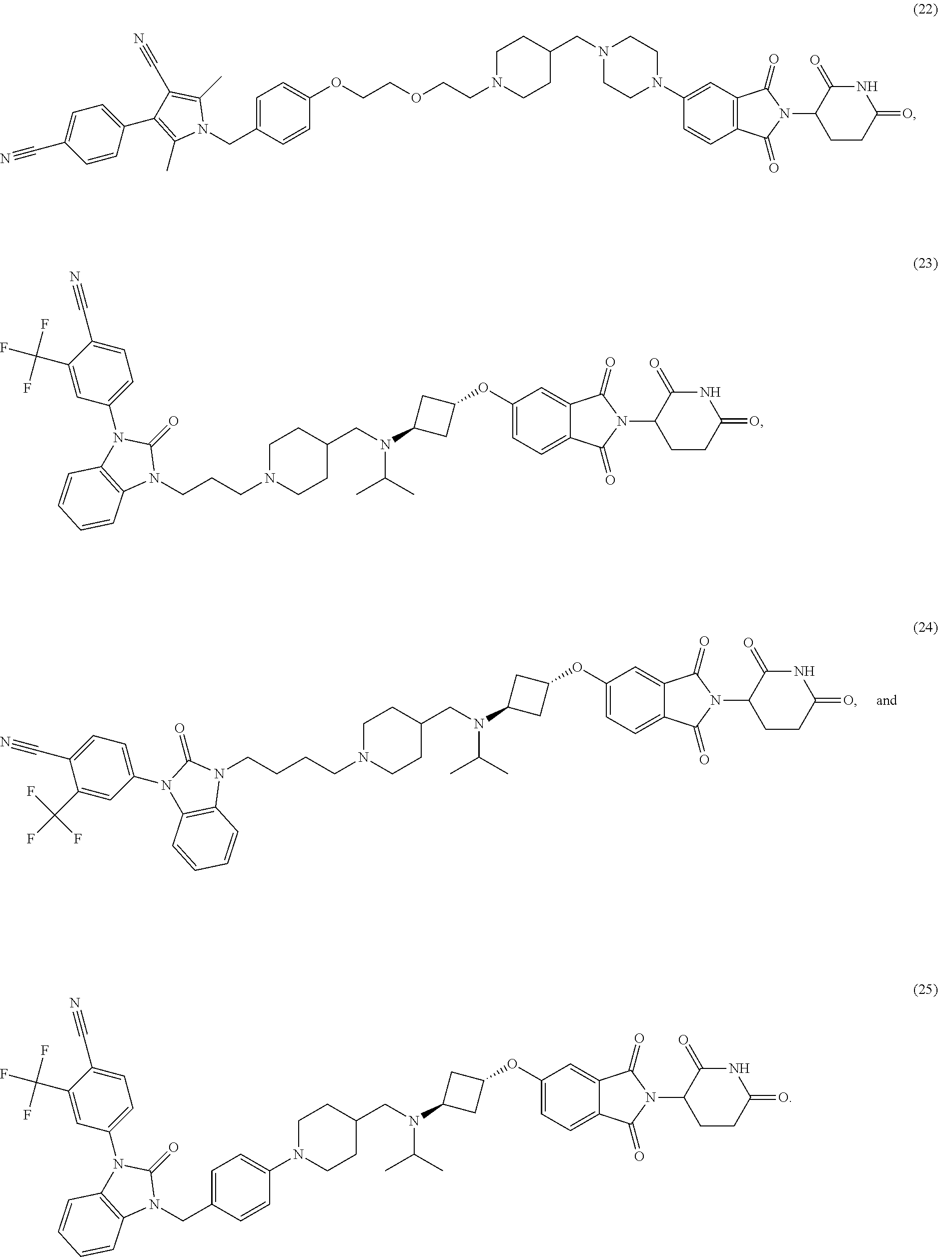 [SMILES: Cc1c(C#N)c(-c2ccc(C#N)cc2)c(C)n1Cc1ccc(OCCOCCN2CCC(CN3CCN(c4ccc5c(c4)C(=O)N(C4CCC(=O)NC4=O)C5=O)CC3)CC2)cc1 ; CC(C)N(CC1CCN(CCCn2c(=O)n(-c3ccc(C#N)c(C(F)(F)F)c3)c3ccccc32)CC1)[C@H]1C[C@H](Oc2ccc3c(c2)C(=O)N(C2CCC(=O)NC2=O)C3=O)C1 ; CC(C)N(CC1CCN(CCCCn2c(=O)n(-c3ccc(C#N)c(C(F)(F)F)c3)c3ccccc32)CC1)[C@H]1C[C@H](Oc2ccc3c(c2)C(=O)N(C2CCC(=O)NC2=O)C3=O)C1 ; "and" ; CC(C)N(CC1CCN(c2ccc(Cn3c(=O)n(-c4ccc(C#N)c(C(F)(F)F)c4)c4ccccc43)cc2)CC1)[C@H]1C[C@H](Oc2ccc3c(c2)C(=O)N(C2CCC(=O)NC2=O)C3=O)C1]

11. A composition comprising an effective amount of a bifunctional compound of claim 1, and a pharmaceutically acceptable carrier.

12. The composition of claim 11, wherein the composition further comprises at least one of additional bioactive agent or a second bifunctional compound.

13. The composition of claim 12, wherein the additional bioactive agent is an anti-cancer agent.

14. A composition comprising a pharmaceutically acceptable carrier and an effective amount of at least one compound of claim 1 for treating a disease, a disorder or a symptom casually related to AR in a subject, wherein the composition is effective in treating or ameliorating the disease, disorder, or at least one symptom of the disease or disorder.

15. The composition of claim 14, wherein the disease or disorder is cancer or Kennedy's Disease or both.

16. The composition according to claim 15, wherein the cancer is prostate cancer.

17. The composition according to claim 14, wherein the composition further comprises at least one additional bioactive agent, such as an anti-cancer agent.

18. A method of treating or preventing a disease, a disorder, or symptom associated with AR comprising, providing a patient in need thereof, and administering an effective amount of a compound as described herein or composition comprising the same to the patient, wherein the compound or composition is effective in treating or ameliorating the disease, disorder, or at least one symptom of the disease or disorder.

19. The method of claim 18, wherein the disease or disorder is cancer or Kennedy's Disease or both.

20. The method of claim 19, wherein the cancer is prostate cancer.

21. The method of claim 18, wherein the composition further comprises an effective amount of at least one additional anti-cancer agent.
